(19) United States
(12) Patent Application Publication
(10) Pub. No.: US 2019/0256509 A1
(43) Pub. Date: Aug. 22, 2019

(54) HISTONE DEMETHYLASE INHIBITORS

(71) Applicant: CHRYSALIS, INC., ESCONDIDO, CA (US)

(72) Inventor: Stephen GWALTNEY, Chapel Hill, NC (US)

(21) Appl. No.: 16/307,729

(22) PCT Filed: Jun. 8, 2017
(86) PCT No.: PCT/US2017/036578

§ 371 Date: Dec. 6, 2018

Related U.S. Application Data
(60) Provisional application No. 62/347,353, filed on Jun. 8, 2016.

(51) Int. Cl. C07D 471/04

(52) U.S. Cl. CPC C07D 471/04; A61P 35/00

(57) ABSTRACT

This disclosure relates, inter alia, to compounds that inhibit histone demethylase activity. In particular, the disclosure relates to compounds that inhibit histone lysine demethylase KDM5B, pharmaceutical compositions and methods of use, such as methods of treating cancer using the compounds and pharmaceutical compositions disclosed herein.

### FIELD

This invention relates to the field of cancer treatment.

### BACKGROUND

Histone methylation plays an important role in the epigenetic regulation of a number of diverse biological processes and diseases. Histone lysine demethylases are a class of enzymes that remove methyl groups from mono-, di- or tri-methylated lysine residues of histones to regulate gene expression and modulate chromatin structure.

Histone lysine demethylases are classified into two separate superfamilies based on sequence homology and mechanism of action. The members of the KDM1 (Lysine (K) demethylase 1) superfamily are FAD-dependent amine oxidases, which act on mono-/di-methylated lysine residues, whereas the other histone demethylase superfamily members are Fe(II) and 2-oxoglutarate-dependent enzymes, and share the signature Jumonji C (JmjC) domain. Members of the latter histone lysine demethylase superfamily have been further classified into separate groups based on JmjC sequence homology and other associated motifs (see, e.g., Pedersen and Helin (2010) Trends in Cell Biol. 20:672-677).

KDM5B (JARID1B) is a member of the JmjC histone lysine demethylase superfamily and acts on di- and trimethylated lysine residues of histones, particularly di- and trimethylated lysine 4 in the N-terminal tail of histone H3. KDM5B has been reported to be overexpressed in a number of cancers, including breast, prostate, testicular, ovarian, leukemia and bladder carcinoma, and KDM5B activity is reported to be required for continued growth of melanoma (see, e.g., H\acute{ø}fedlt et al., (2013) Nature Rev Drug Disc., Published on line Nov. 13, 2013 doi:10.1038/nrd4154).

With increasing evidence that histone lysine demethylases, including KDM5B, play a critical role in a diverse set of cancers and diseases, a variety of histone demethylase inhibitors have been reported in the literature (e.g., see Lizcano and Garcia (2012) Pharmaceuticals 5:963-990). Inhibitors of KDM5B and other Jumonji C superfamily members compete with the 2-oxoglutrate co-factor and bind to the catalytic region containing Fe(II) to block demethylation. But KDM5B inhibitors have yet to successfully advance into human clinical trials.

### SUMMARY

In certain aspects, compounds are provided that inhibit KDM5B activity. In certain embodiments, the compounds are represented by formula (I):

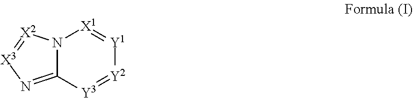

or a pharmaceutical salt thereof, wherein:

X¹ and X² are each independently N, CR¹, or CR³, wherein at least one of X¹ or X² is CR¹; and X³ is N or CR³;

Y¹ and Y² are each independently N, CR² or CR³, wherein at least one of Y¹ or Y² is CR² and R² is -L-R⁵, and Y³ is N or CR³;

R¹ is hydroxyl, cyano, —COOR⁴, —C(O)N(H)CN, —C(O)N(H)OH, or tetrazolyl, and R² is hydrogen, halogen, alkyl, alkoxy, or -L-R⁵;

L is a bond or —(CH₂)ₘ—W—(CH₂)ₙ—, and W is absent, NR⁴, O, C(O), C(O)NR⁴; NR⁴C(O), S, SO, SO₂, NR⁴SO₂ or SO₂NR⁴;

R³ is hydrogen, halogen, alkyl or alkoxy, and R⁴ is hydrogen or alkyl;

R⁵ is hydrogen, carbocyclyl, heterocyclyl, aryl, or heteroaryl, wherein each of the carbocyclyl, heterocyclyl, aryl, or heteroaryl may be optionally substituted with one or more R⁶;

R⁶ is selected from the group consisting of hydroxyl, hydroxylalkyl, alkyl, arylalkyl, alkyl sulfonyl, halogen, haloalkyl, alkoxy, haloalkylalkoxy, cyano, acylaminyl, dialkylaminyl, aryl, aryloxy, alkoxyaryl, aryloxyalkyl, heterocyclyl, alkylheterocyclyl and heteroaryl; and

m and n are each independently zero or an integer between one and three.

In other aspects, pharmaceutical compositions are provided comprising a therapeutically effective amount of a compound disclosed herein and a pharmaceutically acceptable excipient.

In yet other aspects, methods for inhibiting histone demethylase activity in a cell or methods for treating cancer in a patient are provided comprising administering a therapeutically effective amount of a compound or pharmaceutical composition disclosed herein to a cell or to a patient in need thereof.

Numerous other aspects are provided in accordance with these and other aspects of the invention. Other features and aspects of the present invention will become more fully apparent from the following detailed description and the appended claims.

### DETAILED DESCRIPTION

As used herein, the word “a” or “plurality” before a noun represents one or more of the particular noun. For example, the phrase “a mammalian cell” represents “one or more mammalian cells.”

As used herein, “KDM5B” refers to a mammalian Jumonji C superfamily histone lysine demethylase which removes methyl groups from tri- and dimethylated lysine4 of the histone H3 protein.

As used herein, a “KDM5B inhibitor” refers to compounds disclosed herein that are represented by formula (I) as described herein. These compounds are able to negatively modulate or to inhibit all or a portion of the enzymatic activity of KDM5B.

The KDM5B can be from any animal that has KDM5B, including from a human.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B-, wherein a is 0 or 1. In such instances, when a is 0 the moiety is B- and when a is 1 the moiety is A-B-. Also, a number of moieties disclosed herein exist in multiple tautomeric forms, all of which are intended to be encompassed by any given tautomeric structure.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, propyl, isopropyl, and cyclopropyl.

The term “azine” refers to aromatic heterocycles containing at least one nitrogen.

The term “alkyl” as employed herein refers to straight and branched chain aliphatic groups having from 1 to 12 carbon atoms (1, 2, 3, 4, 5, 6, 7, 8, 9, 10, 11, or 12), which is optionally substituted with one, two or three substituents. Exemplary alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond (like “C₀” hydrocarbyl).

The term “alkenyl” as used herein means an unsaturated straight or branched chain aliphatic group with one or more carbon-carbon double bonds, having from 2 to 12 carbon atoms (2, 3, 4, 5, 6, 7, 8, 9, 10, 11, or 12), which is optionally substituted with one, two or three substituents. Exemplary alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms (2, 3, 4, 5, 6, 7, 8, 9, 10, 11, or 12), which is optionally substituted with one, two or three substituents. Exemplary alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Exemplary alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “alkoxy” refers to —O-alkyl.

The term “cycloalkyl” as employed herein includes saturated and partially unsaturated cyclic hydrocarbon groups having 3 to 12 carbons (3, 4, 5, 6, 7, 8, 9, 10, 11, or 12), wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteratom selected from the group consisting of O, S, and N.

An “aryl” group is a C₅-C₁₄ aromatic moiety comprising one to three aromatic rings, which is optionally substituted. The aryl group can be a C₆-C₁₀ aryl group. Exemplary aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. In certain embodiments, the aralkyl group is (C₁-C₆)alk(C₆-C₁₀)aryl, including, without limitation, benzyl, phenethyl, and naphthylmethyl.

A “heterocyclyl” or “heterocyclic” group is a ring structure having from about 3 to about 8 atoms, preferably 4 to 7 atoms, wherein one or more atoms are selected from the group consisting of N, O, and S. The heterocyclic group is optionally substituted on carbon at one or more positions. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Exemplary heterocyclic groups include, without limitation, epoxy, azetidinyl, aziridinyl, tetrahydrofuranyl, pyrrolidinyl, piperidinyl, piperazinyl, thiazolidinyl, oxazolidinyl, oxazolidinonyl, and morpholino. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 pi electrons shared in a cyclic array; and having, in addition to carbon atoms, from one to three heteroatoms per ring selected from the group consisting of N, O, and S. A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl, quinazolinylmethyl, quinolinylmethyl, quinolinylethyl, benzofuranylmethyl, indolinylethyl, isoquinolinylmethyl, isoindolylmethyl, cinnolinylmethyl, and benzothiophenylethyl. Specifically excluded from the scope of this term are compounds having adjacent annular O and/or S atoms.

An “arylene,” “heteroarylene,” or “heterocyclylene” group is an aryl, heteroaryl, or heterocyclyl group, as defined hereinabove, that is positioned between and serves to connect two other chemical groups.

Exemplary heterocyclyls and heteroaryls include, but are not limited to, acridinyl, azocinyl, azetidinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., cycloalkyl, hydrocarbyl, aryl, heteroaryl, heterocyclic, urea, etc.) is described as “optionally substituted” it is meant that the group optionally has from one to four (1, 2, 3, or 4) non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

A “halohydrocarbyl” is a hydrocarbyl moiety in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NR₃₀, R₃₁, alkylamino, arylamino, and cyclic amino groups. The term “ureido” as employed herein refers to a substituted or unsubstituted urea moiety.

The term “radical” as used herein means a chemical moiety comprising one or more unpaired electrons.

A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2-fluor-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—).

As used herein, an “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

As used herein, a “therapeutically effective amount of a compound” is an amount that is sufficient to ameliorate, or in some manner reduce, a symptom or stop or reverse progression of a condition, or negatively modulate or inhibit the activity of KDM5B. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Compounds

In certain aspects, compounds are provided that inhibit KDM5 activity. In certain embodiments, the compounds are represented by formula (I):

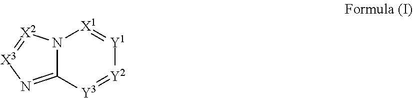

or a pharmaceutical salt thereof, wherein:

X¹ and X² are each independently N, CR¹, or CR³, wherein at least one of X¹ or X² is CR¹; and X³ is N or CR³;

Y¹ and Y² are each independently N, CR² or CR³, wherein at least one of Y¹ or Y² is CR² and R² is -L-R⁵, and Y³ is N or CR³;

R¹ is hydroxyl, cyano, —COOR⁴, —C(O)N(H)CN, —C(O)N(H)OH, or tetrazolyl, and R² is hydrogen, halogen, alkyl alkoxy, or -L-R⁵;

L is a bond or —(CH₂)ₘ—W—(CH₂)ₙ—, and W is absent, NR⁴, O, C(O), C(O)NR⁴; NR⁴C(O), S, SO, SO₂, NR⁴SO₂ or SO₂NR⁴;

R³ is hydrogen, halogen, alkyl or alkoxy, and R⁴ is hydrogen or alkyl;

R⁵ is hydrogen, carbocyclyl, heterocyclyl, aryl, or heteroaryl, wherein each of the carbocyclyl, heterocyclyl, aryl, or heteroaryl may be optionally substituted with one or more R⁶;

R⁶ is selected from the group consisting of hydroxyl, hydroxylalkyl, alkyl, arylalkyl, alkyl sulfonyl, halogen, haloalkyl, alkoxy, haloalkylalkoxy, cyano, acylaminyl, dialkylaminyl, aryl, aryloxy, alkoxyaryl, arylhydroxyalkyl, heterocyclyl, alkylheterocyclyl and heteroaryl; and

m and n are each independently zero or an integer between one and three.

In certain embodiments, X² is CR¹.

In certain other embodiments, X¹ is CR¹, and R¹ is tetrazolyl or —COOR⁴. In certain preferred embodiments, R⁴ is hydrogen.

In certain embodiments, R¹ is —COOH, L is —NR⁴CH₂— and R⁵ is aryl or hetreroaryl. In certain further embodiments, the aryl is selected from the group consisting of phenyl, naphthyl and tetrahydronaphthyl, wherein each is optionally substituted with one or more R⁶. R⁶ aryl substituents include, for example, hydroxyl, hydroxylalkyl, alkyl, arylalkyl, alkylsulfonyl, halogen, haloalkyl, alkoxy, haloalkylalkoxy, cyano, acylaminyl, dialkylaminyl, aryl, aryloxy, alkoxyaryl, arylhydroxyalkyl, heterocyclyl, alkylheterocyclyl and heteroaryl.

In yet other embodiments, R⁵ is a heteroaryl selected from the group consisting of benzofuranyl, benzothophenyl, benzimidazolone, dihydrobenzodioxinyl and dihydroisoquinolinyl, each optionally substituted with one or more R⁶. R⁶ heteroaryl substituents include, for example, alkyl and halogen.

In certain embodiments, exemplary compounds of formula (I) are selected from the group consisting of:

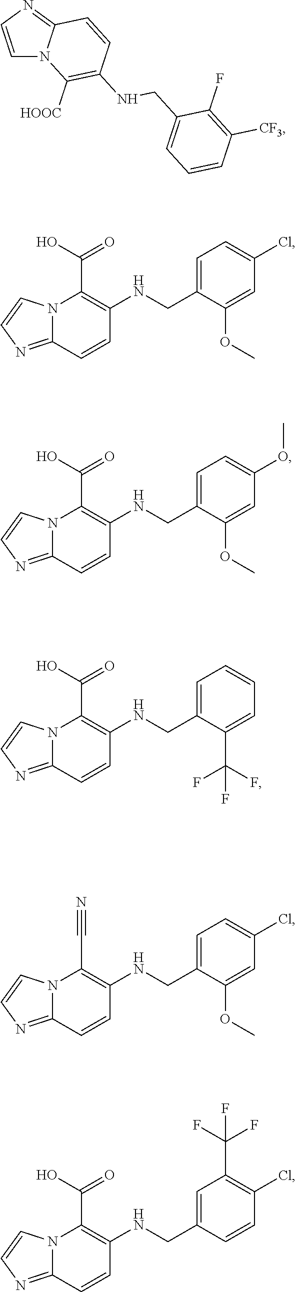 [SMILES: O=C(O)c1c(NCc2cccc(C(F)(F)F)c2F)ccc2nccn12 ; COc1cc(Cl)ccc1CNc1ccc2nccn2c1C(=O)O ; COc1ccc(CNc2ccc3nccn3c2C(=O)O)c(OC)c1 ; O=C(O)c1c(NCc2ccccc2C(F)(F)F)ccc2nccn12 ; COc1cc(Cl)ccc1CNc1ccc2nccn2c1C#N ; O=C(O)c1c(NCc2ccc(Cl)c(C(F)(F)F)c2)ccc2nccn12]

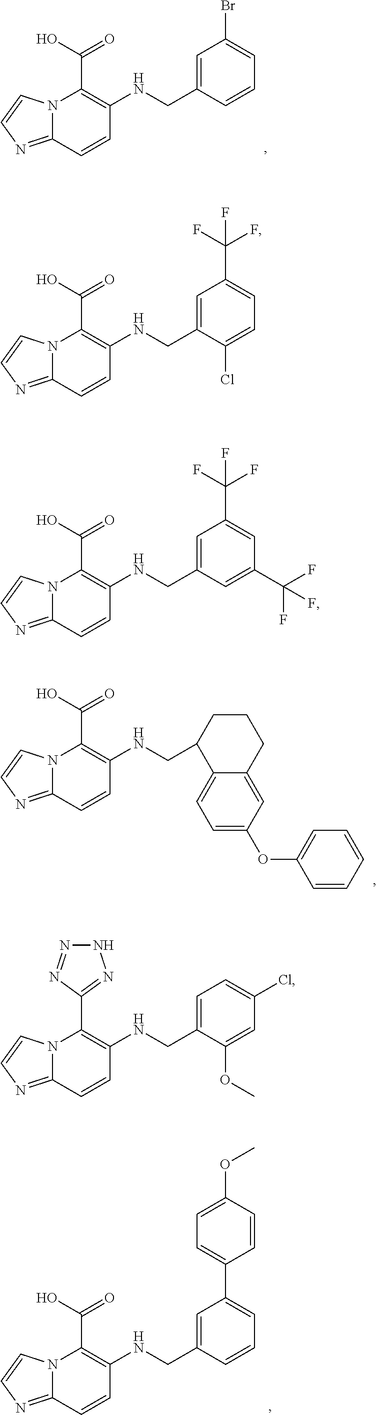 [SMILES: O=C(O)c1c(NCc2cccc(Br)c2)ccc2nccn12 ; O=C(O)c1c(NCc2cc(C(F)(F)F)ccc2Cl)ccc2nccn12 ; O=C(O)c1c(NCc2cc(C(F)(F)F)cc(C(F)(F)F)c2)ccc2nccn12 ; O=C(O)c1c(NCC2CCCc3cc(Oc4ccccc4)ccc32)ccc2nccn12 ; COc1cc(Cl)ccc1CNc1ccc2nccn2c1-c1nn[nH]n1 ; COc1ccc(-c2cccc(CNc3ccc4nccn4c3C(=O)O)c2)cc1]

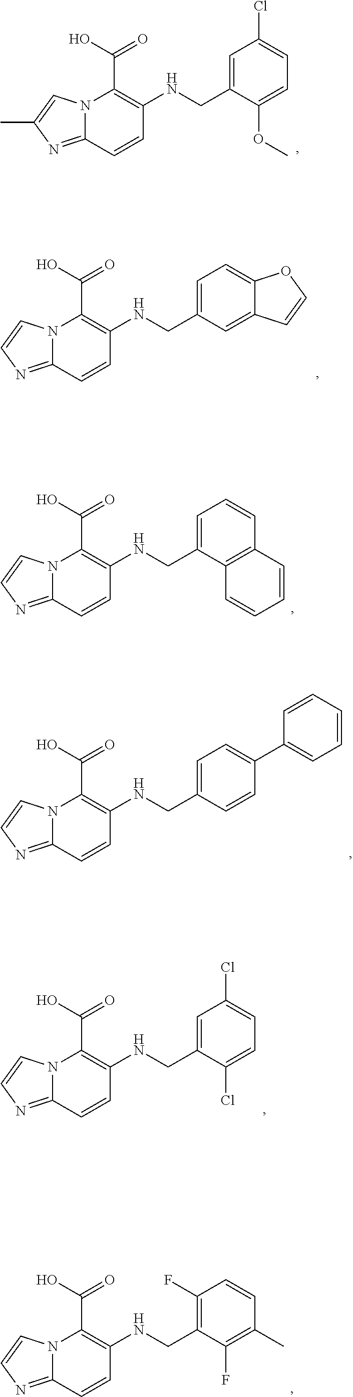

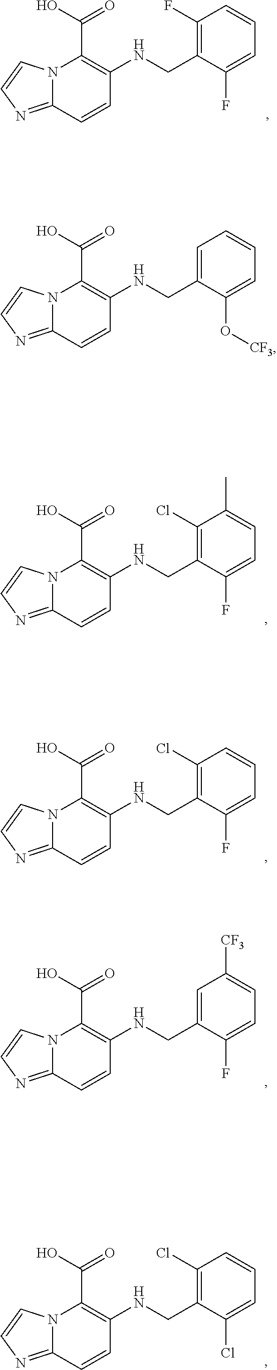 [SMILES: O=C(O)c1c(NCc2c(F)cccc2F)ccc2nccn12 ; O=C(O)c1c(NCc2ccccc2OC(F)(F)F)ccc2nccn12 ; Cc1ccc(F)c(CNc2ccc3nccn3c2C(=O)O)c1Cl ; O=C(O)c1c(NCc2c(F)cccc2Cl)ccc2nccn12 ; O=C(O)c1c(NCc2cc(C(F)(F)F)ccc2F)ccc2nccn12 ; O=C(O)c1c(NCc2c(Cl)cccc2Cl)ccc2nccn12]

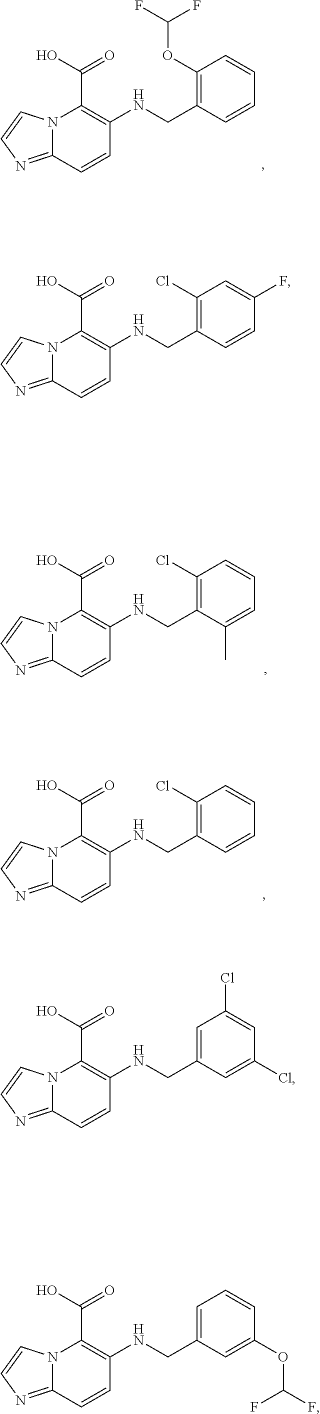 [SMILES: O=C(O)c1c(NCc2ccccc2OC(F)F)ccc2nccn12 ; O=C(O)c1c(NCc2ccc(F)cc2Cl)ccc2nccn12 ; Cc1cccc(Cl)c1CNc1ccc2nccn2c1C(=O)O ; O=C(O)c1c(NCc2ccccc2Cl)ccc2nccn12 ; O=C(O)c1c(NCc2cc(Cl)cc(Cl)c2)ccc2nccn12 ; O=C(O)c1c(NCc2cccc(OC(F)F)c2)ccc2nccn12]

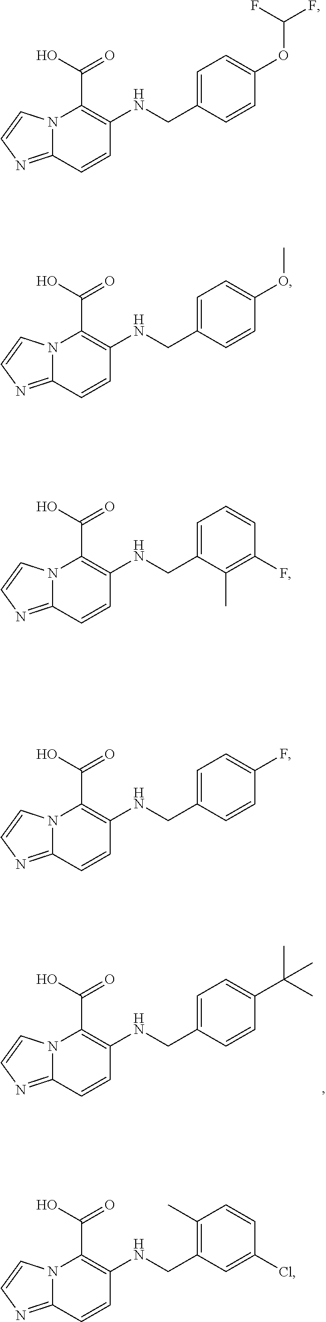 [SMILES: O=C(O)c1c(NCc2ccc(OC(F)F)cc2)ccc2nccn12 ; COc1ccc(CNc2ccc3nccn3c2C(=O)O)cc1 ; Cc1c(F)cccc1CNc1ccc2nccn2c1C(=O)O ; O=C(O)c1c(NCc2ccc(F)cc2)ccc2nccn12 ; CC(C)(C)c1ccc(CNc2ccc3nccn3c2C(=O)O)cc1 ; Cc1ccc(Cl)cc1CNc1ccc2nccn2c1C(=O)O]

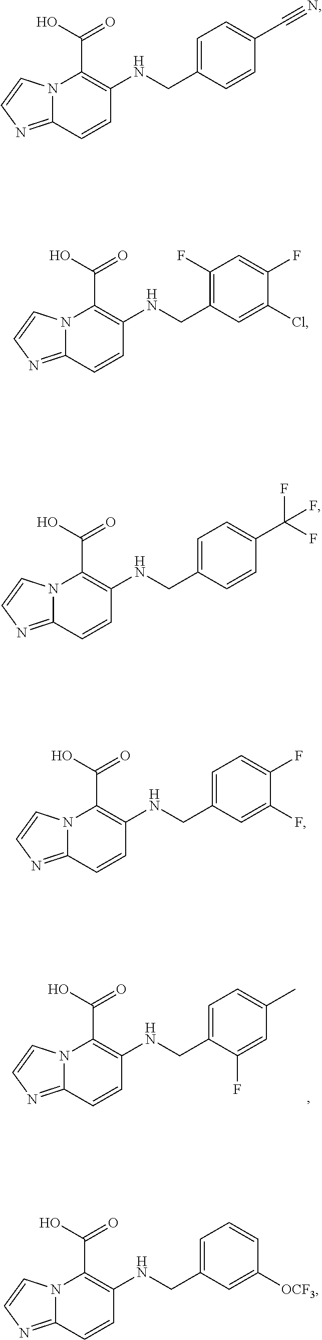 [SMILES: N#Cc1ccc(CNc2ccc3nccn3c2C(=O)O)cc1 ; O=C(O)c1c(NCc2cc(Cl)c(F)cc2F)ccc2nccn12 ; O=C(O)c1c(NCc2ccc(C(F)(F)F)cc2)ccc2nccn12 ; O=C(O)c1c(NCc2ccc(F)c(F)c2)ccc2nccn12 ; Cc1ccc(CNc2ccc3nccn3c2C(=O)O)c(F)c1 ; O=C(O)c1c(NCc2cccc(OC(F)(F)F)c2)ccc2nccn12]

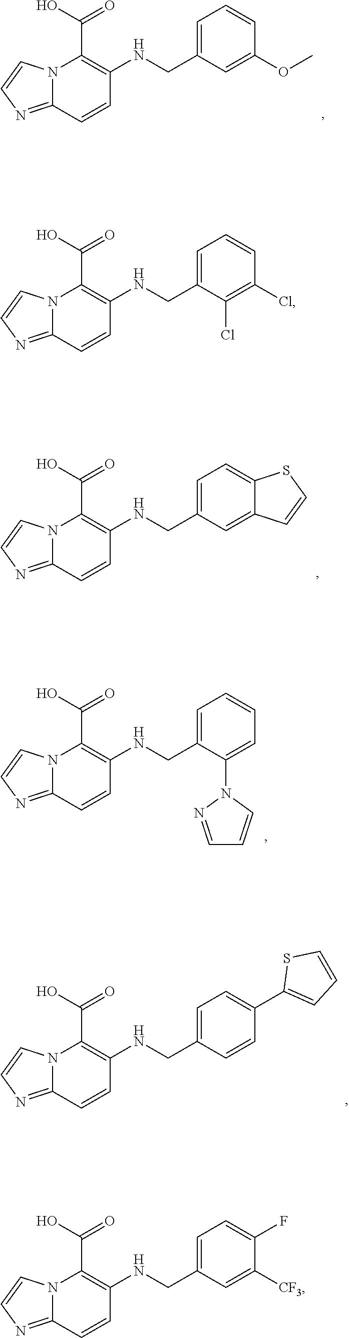 [SMILES: COc1cccc(CNc2ccc3nccn3c2C(=O)O)c1 ; O=C(O)c1c(NCc2cccc(Cl)c2Cl)ccc2nccn12 ; O=C(O)c1c(NCc2ccc3sccc3c2)ccc2nccn12 ; O=C(O)c1c(NCc2ccccc2-n2cccn2)ccc2nccn12 ; O=C(O)c1c(NCc2ccc(-c3cccs3)cc2)ccc2nccn12 ; O=C(O)c1c(NCc2ccc(F)c(C(F)(F)F)c2)ccc2nccn12]

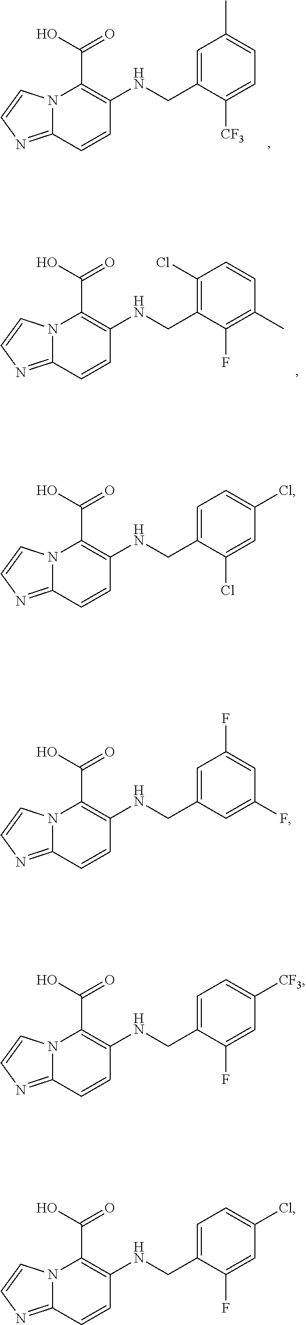 [SMILES: Cc1ccc(C(F)(F)F)c(CNc2ccc3nccn3c2C(=O)O)c1 ; Cc1ccc(Cl)c(CNc2ccc3nccn3c2C(=O)O)c1F ; O=C(O)c1c(NCc2ccc(Cl)cc2Cl)ccc2nccn12 ; O=C(O)c1c(NCc2cc(F)cc(F)c2)ccc2nccn12 ; O=C(O)c1c(NCc2ccc(C(F)(F)F)cc2F)ccc2nccn12 ; O=C(O)c1c(NCc2ccc(Cl)cc2F)ccc2nccn12]

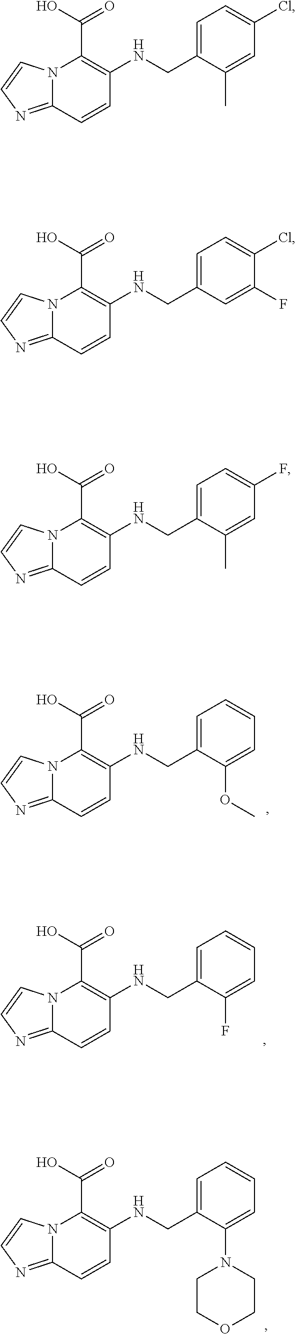 [SMILES: Cc1cc(Cl)ccc1CNc1ccc2nccn2c1C(=O)O ; O=C(O)c1c(NCc2ccc(Cl)c(F)c2)ccc2nccn12 ; Cc1cc(F)ccc1CNc1ccc2nccn2c1C(=O)O ; COc1ccccc1CNc1ccc2nccn2c1C(=O)O ; O=C(O)c1c(NCc2ccccc2F)ccc2nccn12 ; O=C(O)c1c(NCc2ccccc2N2CCOCC2)ccc2nccn12]

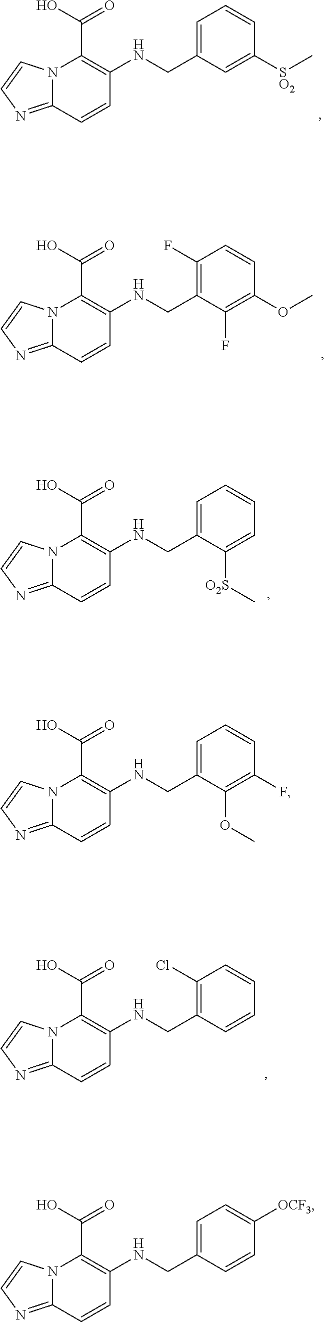 [SMILES: CS(=O)(=O)c1cccc(CNc2ccc3nccn3c2C(=O)O)c1 ; COc1ccc(F)c(CNc2ccc3nccn3c2C(=O)O)c1F ; CS(=O)(=O)c1ccccc1CNc1ccc2nccn2c1C(=O)O ; COc1c(F)cccc1CNc1ccc2nccn2c1C(=O)O ; O=C(O)c1c(NCc2ccccc2Cl)ccc2nccn12 ; O=C(O)c1c(NCc2ccc(OC(F)(F)F)cc2)ccc2nccn12]

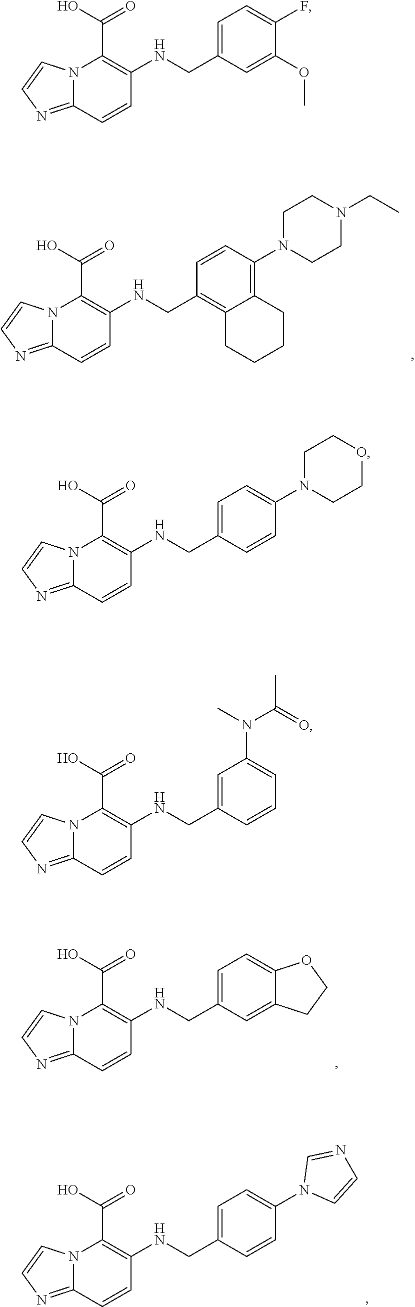 [SMILES: COc1cc(CNc2ccc3nccn3c2C(=O)O)ccc1F ; CCN1CCN(c2ccc(CNc3ccc4nccn4c3C(=O)O)c3c2CCCC3)CC1 ; O=C(O)c1c(NCc2ccc(N3CCOCC3)cc2)ccc2nccn12 ; CC(=O)N(C)c1cccc(CNc2ccc3nccn3c2C(=O)O)c1 ; O=C(O)c1c(NCc2ccc3c(c2)CCO3)ccc2nccn12 ; O=C(O)c1c(NCc2ccc(-n3ccnc3)cc2)ccc2nccn12]

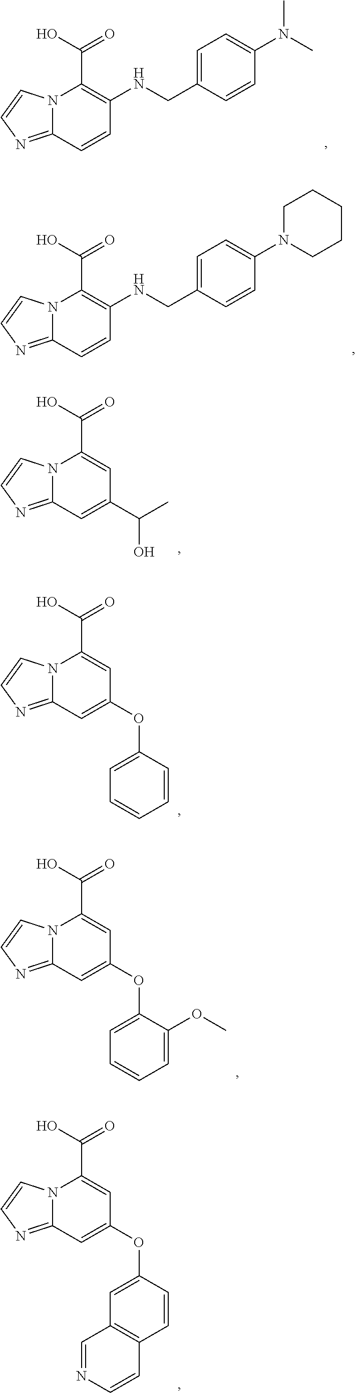 [SMILES: CN(C)c1ccc(CNc2ccc3nccn3c2C(=O)O)cc1 ; O=C(O)c1c(NCc2ccc(N3CCCCC3)cc2)ccc2nccn12 ; CC(O)c1cc(C(=O)O)n2ccnc2c1 ; O=C(O)c1cc(Oc2ccccc2)cc2nccn12 ; COc1ccccc1Oc1cc(C(=O)O)n2ccnc2c1 ; O=C(O)c1cc(Oc2ccc3ccncc3c2)cc2nccn12]

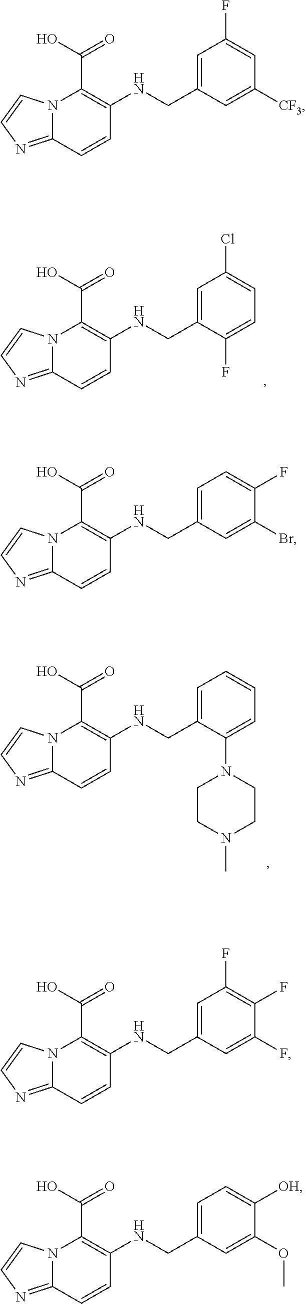 [SMILES: O=C(O)c1c(NCc2cc(F)cc(C(F)(F)F)c2)ccc2nccn12 ; O=C(O)c1c(NCc2cc(Cl)ccc2F)ccc2nccn12 ; O=C(O)c1c(NCc2ccc(F)c(Br)c2)ccc2nccn12 ; CN1CCN(c2ccccc2CNc2ccc3nccn3c2C(=O)O)CC1 ; O=C(O)c1c(NCc2cc(F)c(F)c(F)c2)ccc2nccn12 ; COc1cc(CNc2ccc3nccn3c2C(=O)O)ccc1O]

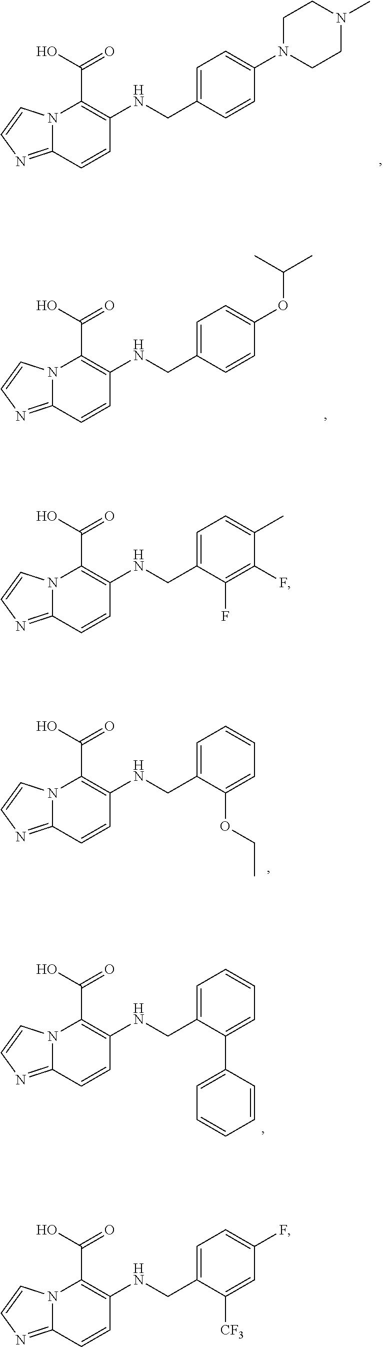 [SMILES: CN1CCN(c2ccc(CNc3ccc4nccn4c3C(=O)O)cc2)CC1 ; CC(C)Oc1ccc(CNc2ccc3nccn3c2C(=O)O)cc1 ; Cc1ccc(CNc2ccc3nccn3c2C(=O)O)c(F)c1F ; CCOc1ccccc1CNc1ccc2nccn2c1C(=O)O ; O=C(O)c1c(NCc2ccccc2-c2ccccc2)ccc2nccn12 ; O=C(O)c1c(NCc2ccc(F)cc2C(F)(F)F)ccc2nccn12]

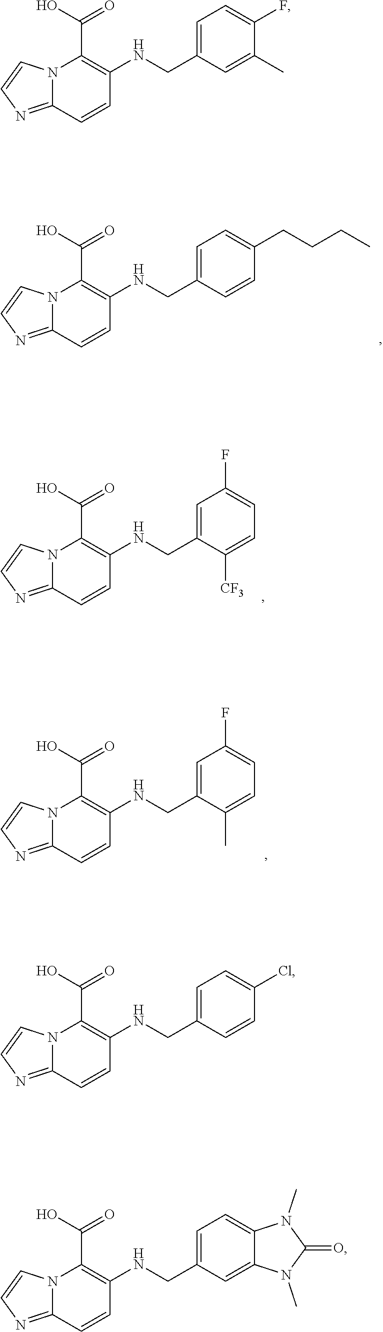 [SMILES: Cc1cc(CNc2ccc3nccn3c2C(=O)O)ccc1F ; CCCCc1ccc(CNc2ccc3nccn3c2C(=O)O)cc1 ; O=C(O)c1c(NCc2cc(F)ccc2C(F)(F)F)ccc2nccn12 ; Cc1ccc(F)cc1CNc1ccc2nccn2c1C(=O)O ; O=C(O)c1c(NCc2ccc(Cl)cc2)ccc2nccn12 ; Cn1c(=O)n(C)c2cc(CNc3ccc4nccn4c3C(=O)O)ccc21]

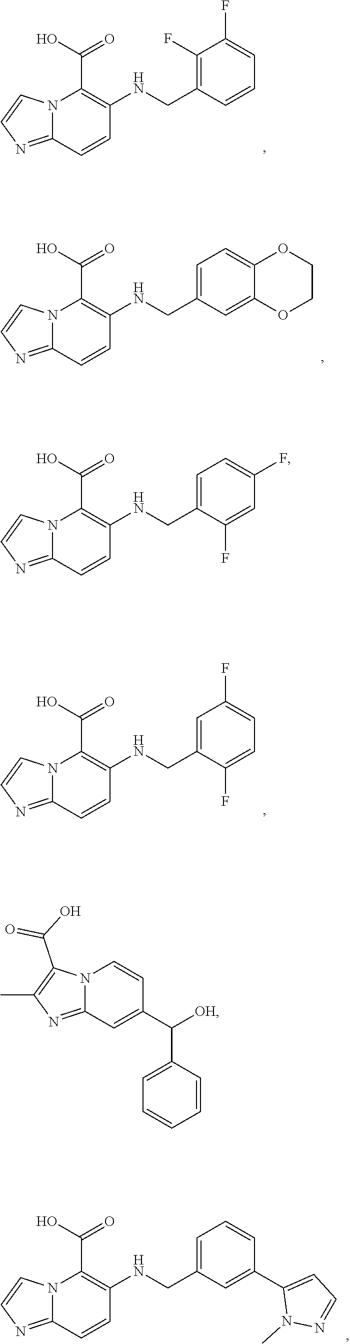 [SMILES: O=C(O)c1c(NCc2cccc(F)c2F)ccc2nccn12 ; O=C(O)c1c(NCc2ccc3c(c2)OCCO3)ccc2nccn12 ; O=C(O)c1c(NCc2ccc(F)cc2F)ccc2nccn12 ; O=C(O)c1c(NCc2cc(F)ccc2F)ccc2nccn12 ; Cc1nc2cc(C(O)c3ccccc3)ccn2c1C(=O)O ; Cn1nccc1-c1cccc(CNc2ccc3nccn3c2C(=O)O)c1]

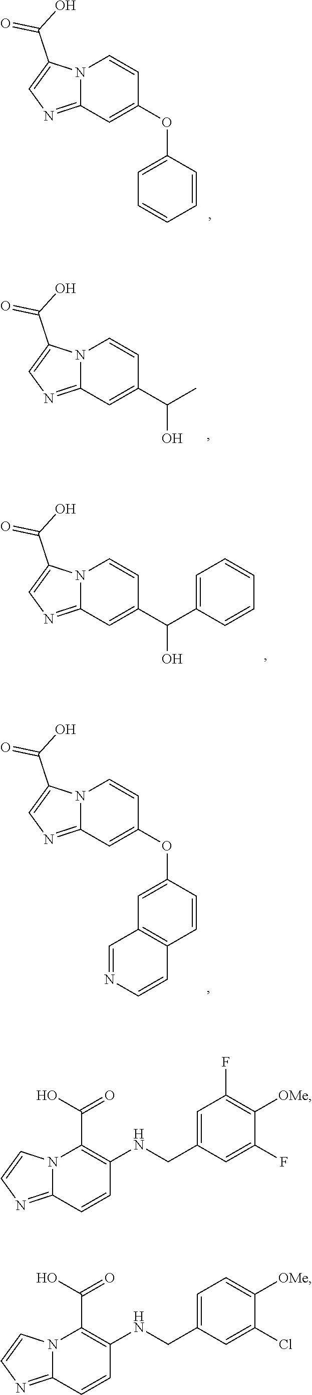 [SMILES: O=C(O)c1cnc2cc(Oc3ccccc3)ccn12 ; CC(O)c1ccn2c(C(=O)O)cnc2c1 ; O=C(O)c1cnc2cc(C(O)c3ccccc3)ccn12 ; O=C(O)c1cnc2cc(Oc3ccc4ccncc4c3)ccn12 ; COc1c(F)cc(CNc2ccc3nccn3c2C(=O)O)cc1F ; COc1ccc(CNc2ccc3nccn3c2C(=O)O)cc1Cl]

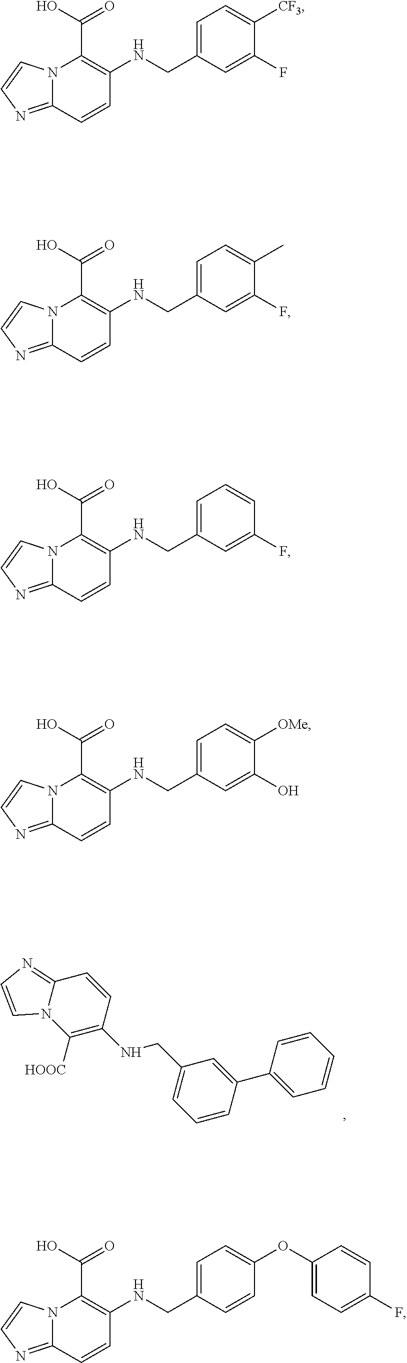 [SMILES: O=C(O)c1c(NCc2ccc(C(F)(F)F)c(F)c2)ccc2nccn12 ; Cc1ccc(CNc2ccc3nccn3c2C(=O)O)cc1F ; O=C(O)c1c(NCc2cccc(F)c2)ccc2nccn12 ; COc1ccc(CNc2ccc3nccn3c2C(=O)O)cc1O ; O=C(O)c1c(NCc2cccc(-c3ccccc3)c2)ccc2nccn12 ; O=C(O)c1c(NCc2ccc(Oc3ccc(F)cc3)cc2)ccc2nccn12]

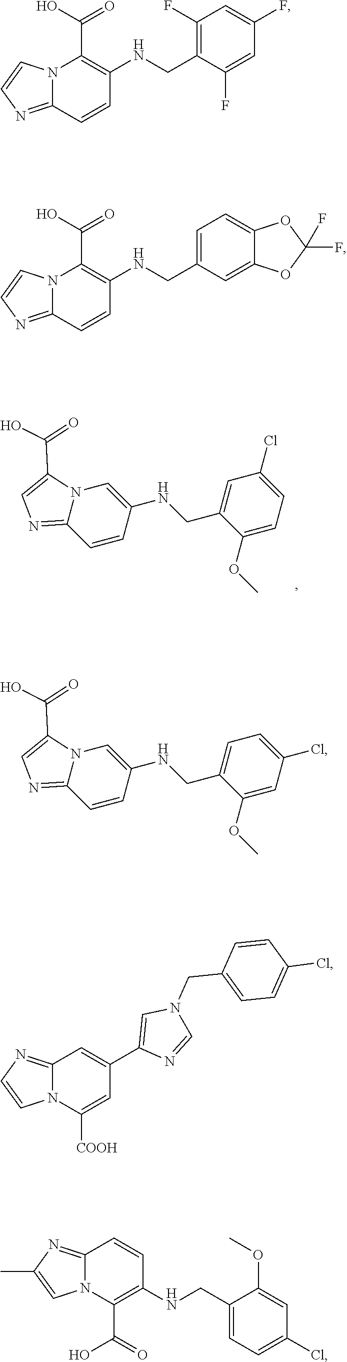 [SMILES: O=C(O)c1c(NCc2c(F)cc(F)cc2F)ccc2nccn12 ; O=C(O)c1c(NCc2ccc3c(c2)OC(F)(F)O3)ccc2nccn12 ; COc1ccc(Cl)cc1CNc1ccc2ncc(C(=O)O)n2c1 ; COc1cc(Cl)ccc1CNc1ccc2ncc(C(=O)O)n2c1 ; O=C(O)c1cc(-c2cn(Cc3ccc(Cl)cc3)cn2)cc2nccn12 ; COc1cc(Cl)ccc1CNc1ccc2nc(C)cn2c1C(=O)O]

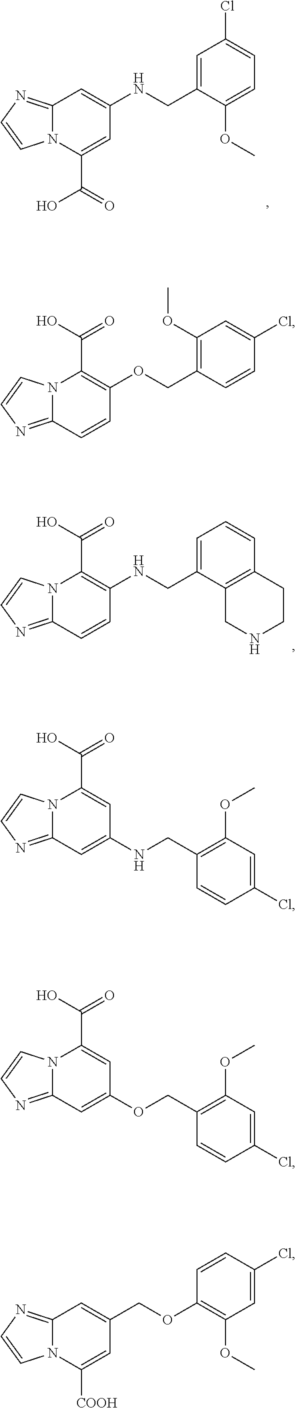 [SMILES: COc1ccc(Cl)cc1CNc1cc(C(=O)O)n2ccnc2c1 ; COc1cc(Cl)ccc1COc1ccc2nccn2c1C(=O)O ; O=C(O)c1c(NCc2cccc3c2CNCC3)ccc2nccn12 ; COc1cc(Cl)ccc1CNc1cc(C(=O)O)n2ccnc2c1 ; COc1cc(Cl)ccc1COc1cc(C(=O)O)n2ccnc2c1 ; COc1cc(Cl)ccc1OCc1cc(C(=O)O)n2ccnc2c1]

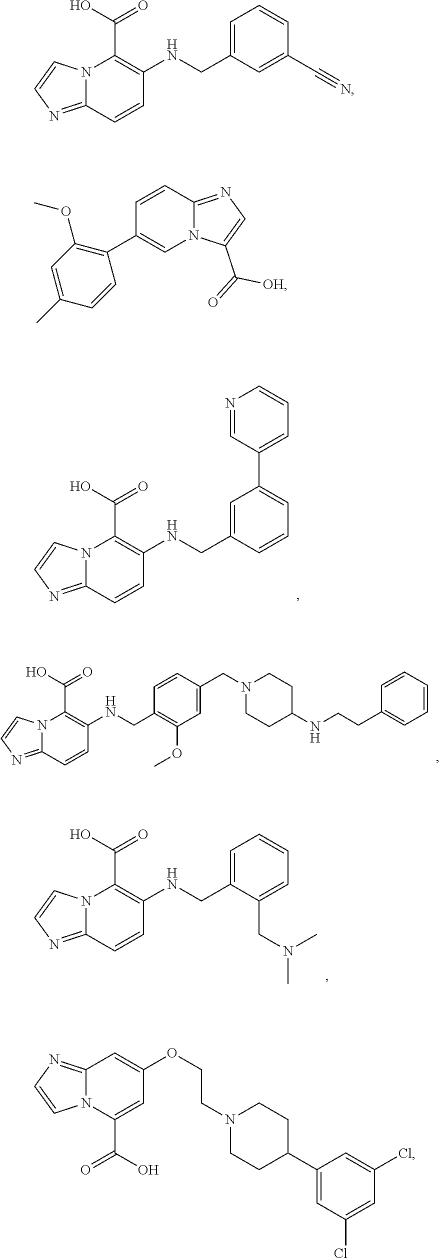 [SMILES: N#Cc1cccc(CNc2ccc3nccn3c2C(=O)O)c1 ; COc1cc(C)ccc1-c1ccc2ncc(C(=O)O)n2c1 ; O=C(O)c1c(NCc2cccc(-c3cccnc3)c2)ccc2nccn12 ; COc1cc(CN2CCC(NCCc3ccccc3)CC2)ccc1CNc1ccc2nccn2c1C(=O)O ; CN(C)Cc1ccccc1CNc1ccc2nccn2c1C(=O)O ; O=C(O)c1cc(OCCN2CCC(c3cc(Cl)cc(Cl)c3)CC2)cc2nccn12]

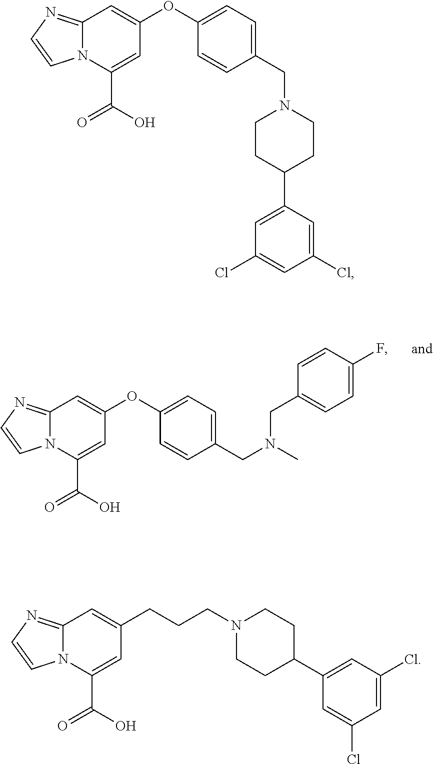 [SMILES: O=C(O)c1cc(Oc2ccc(CN3CCC(c4cc(Cl)cc(Cl)c4)CC3)cc2)cc2nccn12 ; CN(Cc1ccc(F)cc1)Cc1ccc(Oc2cc(C(=O)O)n3ccnc3c2)cc1 ; O=C(O)c1cc(CCCN2CCC(c3cc(Cl)cc(Cl)c3)CC2)cc2nccn12]

The compounds of formula (I) may be formulated into pharmaceutical compositions.

The compounds disclosed herein may have one or more chiral centers and can be synthesized as stereoisomeric mixtures, isomers of identical constitution that differ in the arrangement of their atoms in space. The compounds may be used as mixtures or the individual components/isomers may be separated using reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds disclosed herein may be synthesized using optically pure, chiral reagents and intermediates to prepare individual isomers or enantiomers. Unless otherwise indicated, all chiral (enantiomeric and diastereomeric) and racemic forms are within the scope of the compounds disclosed herein.

Unless otherwise indicated to the contrary, chemical structures, which include one or more stereocenters, illustrated herein without indicating absolute or relative stereochemistry, encompass all possible stereoisomeric forms of the compound (e.g., diastereomers and enantiomers), and mixtures thereof.

### Pharmaceutical Compositions

In another aspect, pharmaceutical compositions are provided comprising a histone demethylase inhibitor disclosed herein and a pharmaceutically acceptable carrier, excipient, or diluent. Compounds disclosed herein may be formulated by any suitable method known in the art and may be prepared for administration by any suitable route, including, without limitation, parenteral, oral, sublingual, transdermal, topical, intranasal, intratracheal, or intrarectal. In certain embodiments, compounds disclosed herein are administered intravenously, such as in a hospital setting. In certain embodiments, the compounds disclosed herein are administered orally.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions disclosed herein may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salts refer to salts that retain the desired biological activity of the above-identified compounds and exhibit minimal or no undesired toxicological effects. Examples of such salts include, but are not limited to, acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In certain embodiments, a dose of the active compound for all of the above-mentioned conditions is in the range from about 0.01 to about 300 mg/kg, from about 0.1 to about 100 mg/kg per day, and from about 0.5 to about 25 mg per kilogram body weight of the recipient per day. A typical topical dosage may range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds may be appropriately formulated by methods known in the art, according to method of use and/or route of administration.

The pharmaceutical compositions comprising compounds disclosed herein may be used in the methods described herein.

### Methods of Use

KDM5B (JARID1B) is a member of the JmjC histone lysine demethylase superfamily and acts on di- and trimethylated lysine residues of histones, particularly di- and trimethylated lysine 4 in the N-terminal tail of histone H3. KDM5B has been reported to be overexpressed in a number of cancers, including breast, prostate, testicular, ovarian, leukemia and bladder carcinoma, and KDM5B activity is reported to be required for continued growth of melanoma (e.g., see H\acute{ø}jfedlt et al., (2013) Nature Rev Drug Disc., Published on line Nov. 13, 2013 doi:10.1038/nrd4154).

With increasing evidence that histone lysine demethylases, including KDM5B, play a critical role in a diverse set of cancers and diseases, a variety of histone demethylase inhibitors have been reported in the literature (e.g., see Lizcano and Garcia (2012) Pharmaceuticals 5:963-990). Inhibitors of KDM5B and other Jumonji C superfamily members compete with the 2-oxoglutrate co-factor and bind to the catalytic region containing Fe(II) to block demethylation.

In yet another aspect, methods are provided for inhibiting KDM5B activity in a cell, comprising contacting the cell in which inhibition of KDM5B activity is desired with a therapeutically effective amount of a compound of formula (I), pharmaceutically acceptable salts thereof or pharmaceutical compositions containing the compound or pharmaceutically acceptable salt thereof.

One use for the compounds, compositions, and methods disclosed herein is for inhibiting KDM5B activity in a cell.

In certain embodiments, a cell in which inhibition of KDM5B activity is desired is contacted with a therapeutically effective amount of a compound of formula (I) to negatively modulate the activity of KDM5B. In other embodiments, a therapeutically effective amount of pharmaceutically acceptable salt or pharmaceutical compositions containing the compound of formula (I) may be used.

By negatively modulating the activity of KDM5B, particularly in cases for cells overexpressing the KDM5B enzyme or somatic mutations that activate the KDM5B enzyme, the methods are designed to restore normal cellular transcription expression patterns, e.g., by altering the methylation pattern of H3K4 to inhibit undesired cellular proliferation resulting from enhanced KDM5B activity and/or expression within the cell. The cells may be contacted in a single dose or multiple doses in accordance with a particular treatment regimen to effect the desired negative modulation of KDM5B. The inhibition of cellular proliferation and KDM5B-dependent demethylation of histone H3K4 may be monitored in the cell using well known methods to assess the effectiveness of treatment and dosages may be adjusted accordingly by the attending medical practitioner.

Methods of determining inhibition of KDM5B are known in the art.

In another aspect, methods are provided of treating cancer comprising administering to a patient having cancer a therapeutically effective amount of a compound of formula (I), pharmaceutically acceptable salts thereof or pharmaceutical compositions comprising the compound or pharmaceutically acceptable salts thereof.

The compositions and methods provided herein may be used for the treatment of a wide variety of cancer, including tumors such as prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Breast; Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

In certain aspects, methods are provided of treating a patient with a cancer in which at least some of the cancerous cells are inappropriately expressing KDM5B, including over-expressing KDM5B, comprising administering to a patient having a cancer in which at least some of the cancerous cells are inappropriately expressing KDM5B, including over-expressing KDM5B a therapeutically effective amount of a compound of formula (I), a pharmaceutically acceptable salts thereof or a pharmaceutical composition comprising the compound or a pharmaceutically acceptable salt thereof.

Methods for the diagnosis of cancer (primary or metastatic) are known in the art. Methods for determining whether the cancer has reduced or has been eliminated, the patient has improved, etc. are known in the art.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively. The inhibition of cellular proliferation and KDM5B-dependent demethylation of histone H3K4 may be monitored in the cell using well known methods to assess the effectiveness of treatment, along with other prognostic or biological factors, and dosages may be adjusted accordingly by the attending medical practitioner.

In certain embodiments, the therapeutically effective amount of a compound disclosed herein is between about 0.01 to about 300 mg/kg per day. In further embodiments, the therapeutically effective amount of a compound disclosed herein is between about 0.1 to about 100 mg/kg per day.

The term “effective amount” or “a therapeutically effective amount” refers to an amount of a compound or composition that provides the desired biological, therapeutic, and/or prophylactic result. That result can be reduction, amelioration, palliation, lessening, delaying, and/or alleviation of one or more of the signs, symptoms, or causes of a disease, such as cancer, in a patient, or any other desired alteration of a biological system. An effective amount can be administered in one or more administrations. In certain other embodiments, an “effective amount” or “a therapeutically effective amount” is the amount of a compound or composition disclosed herein that improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment. An effective amount can be an amount that causes a cancer to shrink or to be eliminated from a patient. Whether a desired result has been achieved can be determined by methods known in the art.

A compound or a composition disclosed herein can be administered to a patient as a monotherapy. In some embodiments, the methods described herein can include administering to the patient one or more additional treatments, such as one or more additional therapeutic agents.

The additional treatment can be any additional treatment, including experimental treatments. The other treatment can be any treatment, any therapeutic agent, that improves or stabilizes the patient's health. An additional therapeutic agent can be administered prior to, concurrently, or after administration of a compound or composition disclosed herein. An additional agent and a compound or composition disclosed herein can be administered using the same delivery method or route or using a different delivery method or route.

In some embodiments, a compound or composition disclosed herein can be formulated with one or more additional active agents useful for treating cancer in a patient.

When a compound or composition disclosed herein is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of a compound or composition disclosed herein and a sub-therapeutic amount of one or more additional active agents such that the components in total are therapeutically effective for treating a cancer. Methods for determining a therapeutically effective dose of an agent are known in the art.

A patient includes a human patient. A subject and a patient is used interchangeably.

### Reaction Schemes and Examples

The compounds disclosed herein may be prepared using commercially available reagents using the synthetic methods and reaction schemes described herein, or using other reagents and conventional methods well known to those skilled in the art.

For instance, substituted bicyclic compounds of the present invention may be prepared according to the General Reaction Schemes I-VII.

### General Reaction Schemes

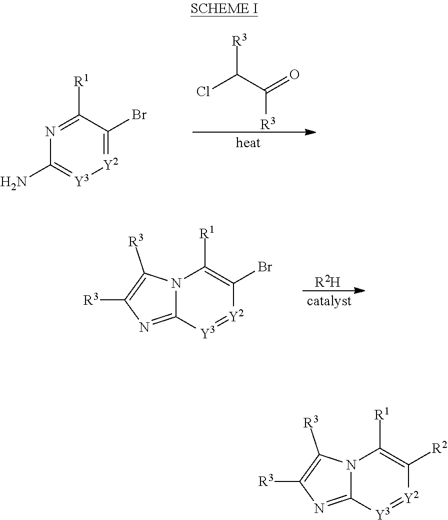

In Scheme I, an amino bromo azine compound is heated with a chlorocarbonyl compound to give the imidazoheteroaryl bicyclic compound. R² is installed via a coupling reaction catalyzed by a transition metal catalyst such as a palladium catalyst.

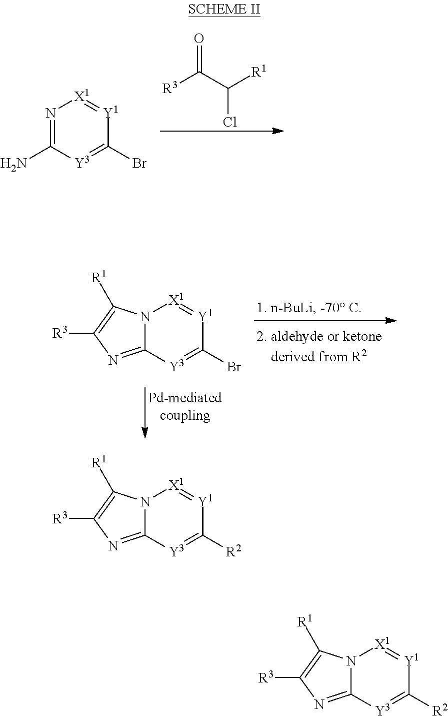

In Scheme II, an amino bromo azine compound is heated with a chlorocarbonyl compound to give the imidazoheteroaryl bicyclic compound. In one instance, R² is installed by metal-halogen exchange followed by treatment of the anion with an aldehyde or ketone. In another instance, R² is installed via a coupling reaction catalyzed by a transition metal catalyst such as a palladium catalyst.

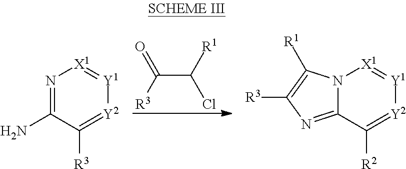

In Scheme III, an amino azine compound is heated with a chlorocarbonyl compound to give the imidazoheteroaryl bicyclic compound.

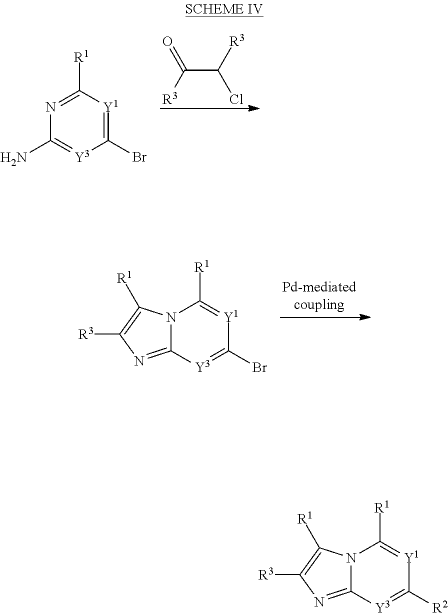

In Scheme IV, an amino bromo azine compound is heated with a chlorocarbonyl compound to give the imidazoheteroaryl bicyclic compound. R² is installed via a coupling reaction catalyzed by a transition metal catalyst such as a palladium catalyst.

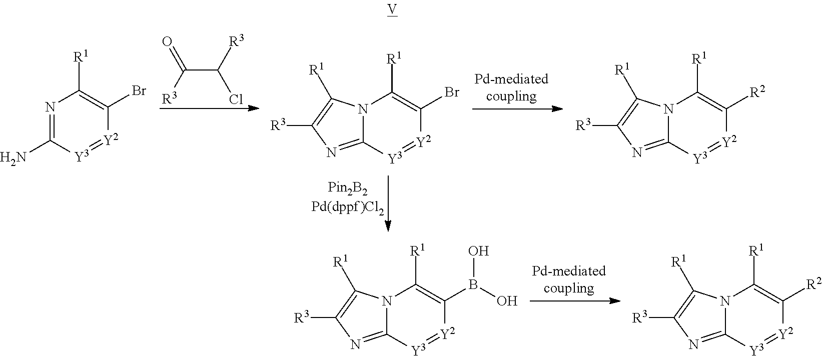

In Scheme V, an amino bromo azine compound is heated with a chlorocarbonyl compound to give the imidazoheteroaryl bicyclic compound. In one instance, R² is installed via a coupling reaction catalyzed by a transition metal catalyst such as a palladium catalyst. In another instance, the bromo imidazopyridine is converted to a boronic acid which is used to install R² via a coupling reaction catalyzed by a palladium catalyst.

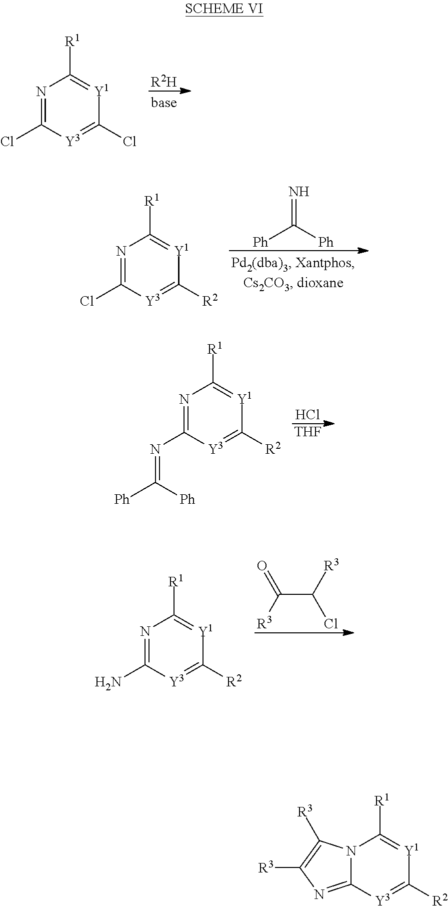

In Scheme VI, a dichloroazine compound is treated with a nucleophilic R²H compound and a base. The resulting chloroazine compound is treated with diphenylmethanimine and a palladium catalyst to give the protected aminoazine compound. Deprotection is affected with acid. The resulting aminoazine compound is heated with a chlorocarbonyl compound to give the imidazoheteroaryl bicyclic compound.

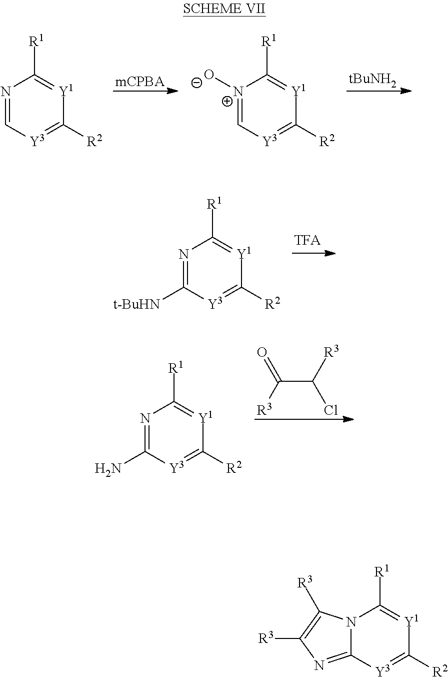

In Scheme VII, an azine compound is treated with an oxidant such as meta-chloroperoxybenzoic acid to give an N-oxide. The N-oxide is treated with t-butylamine to give a protected aminoazine compound. Deprotection is affected with an acid such as trifluoroacetic acid. The resulting aminoazine compound is heated with a chlorocarbonyl compound to give the imidazoheteroaryl bicyclic compound.

The compounds disclosed herein may have one or more chiral centers and can be synthesized as stereoisomeric mixtures, isomers of identical constitution that differ in the arrangement of their atoms in space. The compounds may be used as mixtures or the individual components/isomers may be separated using reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of disclosed herein may be synthesized using optically pure, chiral reagents and intermediates to prepare individual isomers or enantiomers. Unless otherwise indicated, all chiral (enantiomeric and diastereomeric) and racemic forms are within the scope of the invention.

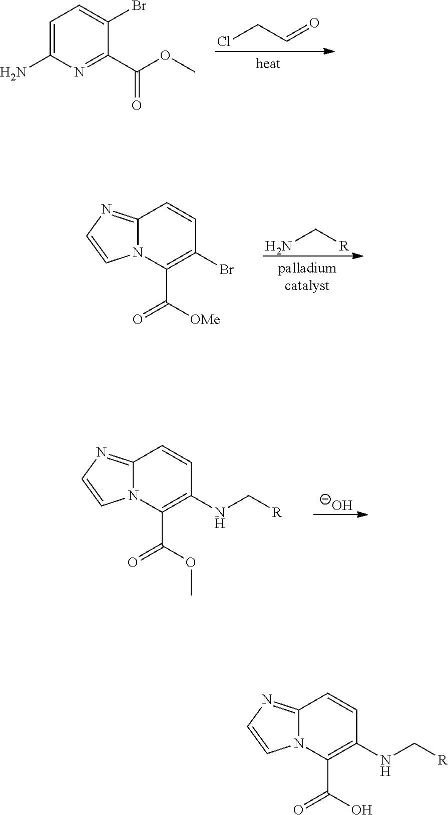

### Scheme for Syntheses of Examples 1-100

### Example 1

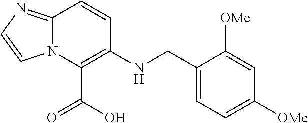 [SMILES: COc1ccc(CNc2ccc3nccn3c2C(=O)O)c(OC)c1]

### Example 1A

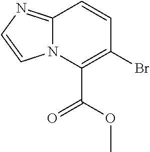

### Example 1A

To a solution of methyl 6-amino-3-bromopicolinate (2.00 g, 8.66 mmol, 1 eq) in EtOH (15 mL) was added chloroacetaldehyde (13.6 g, 86.58 mmol, 50% purity, 10 eq) and NaHCO₃ (1.24 g, 14.72 mmol, 1.7 eq). The mixture was stirred at 95° C. for 5.5 hours (hr). The reaction mixture was filtered and concentrated under reduced pressure. The residue was adjusted to pH=9 with K₂CO₃ aqueous solution and extracted with chloroform. The combined organic layers were washed with brine, and dried over MgSO₄. The concentrated residue was purified by flash chromatography (SiO₂, pentane/ethyl acetate/MeOH=2:1:0.03). Example 1A (2.0 g, 91% yield) was obtained as a brown solid. Mass spectrum (ESI), m/z 255.0 and 257.1[M+H]⁺.

### Example 1B

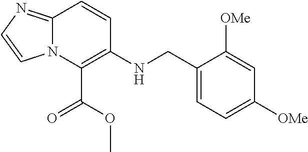 [SMILES: COC(=O)c1c(NCc2ccc(OC)cc2OC)ccc2nccn12]

### Example 1B

A mixture of Example 1A (185 mg, 725 μmol, 1 eq), 2,4-dimethoxybenzyl amine (133 mg, 798 μmol, 1.1 eq), Cs₂CO₃ (236 mg, 725 μmol, 1 eq), Pd₂(dba)₃ (66 mg, 72.5 μmol, 0.1 eq) and xantphos (84 mg, 145 μmol, 0.2 eq) in toluene (3.0 mL) was degassed and purged with N₂ 3 times. The mixture was stirred at 105° C. overnight under a N₂ atmosphere. The reaction mixture was filtered and concentrated under reduced pressure. The residue was purified by flash chromatography (SiO₂, pentane/ethyl acetate/MeOH=1:1:0.1). Example 1B (108 mg, 44% yield) was obtained as yellow oil. Mass spectrum (ESI), m/z 342.2 [M+H]⁺.

### Example 1

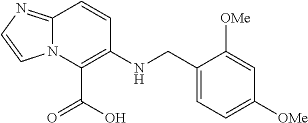 [SMILES: COc1ccc(CNc2ccc3nccn3c2C(=O)O)c(OC)c1]

### Example 1

To a solution of Example 1B (68.5 mg, 200.7 μmol, 1 eq) in MeOH (2 mL) was added 1 M LiOH solution (1.5 ml, 1.5 mmol, 7.5 eq). The mixture was stirred at room temperature overnight. The reaction mixture was adjusted to pH=3 with 1 M HCl solution. The resulting solid was collected by filtration, washed with H₂O and dried in vacuo to give Example 1 (65 mg, 99% yield) as a yellow solid. Mass spectrum (ESI), m/z 328.1 [M+H]⁺.

¹H NMR (500 MHz, DMSO-d₆) ppm 3.73 (s, 3H), 3.81 (s, 3H), 4.40 (s, 2H), 6.45 (dd, J=6.0, 2.5 Hz, 1H), 6.58 (d, J=2.5 Hz, 1H), 7.15 (d, J=8.5 Hz, 1H), 7.29 (d, J=10 Hz, 1H), 7.64-7.68 (m, 2H), 8.97 (br. s., 1H).

The Examples in the following Table 1 were prepared according to the procedures used in Example 1.

TABLE 1
Exemplary Compounds of Formula (I)

| Example | | | |
|---|---|---|---|
| # | Structure | MS data | ¹H NMR data |
| 2 | 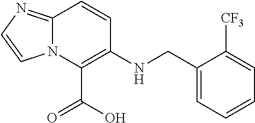 [SMILES: O=C(O)c1c(NCc2ccccc2C(F)(F)F)ccc2nccn12] | (ESI), m/z 336.1 [M + H]⁺; | (500 MHz, DMSO-d₆) ppm 4.70 (s, 2 H), 7.29 (d, J = 10 Hz, 1 H), 7.64-7.68 (m, 6 H), 9.06 (br. s., 1 H). |
| 3 | 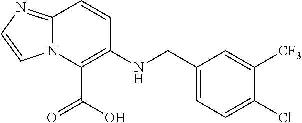 [SMILES: O=C(O)c1c(NCc2ccc(Cl)c(C(F)(F)F)c2)ccc2nccn12] | (ESI), m/z 370.1 [M + H]⁺; | (500 MHz, DMSO-d₆) ppm 3.91 (s, 2 H), 7.25 (d, J = 8 Hz, 1 H), 7.62-7.70 (m, 5 H), 7.84 (s, 1 H), 9.06 (br. s., 1 H). |
| 4 | 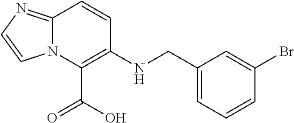 [SMILES: O=C(O)c1c(NCc2cccc(Br)c2)ccc2nccn12] | (ESI), m/z 346.1 and 348.1 [M + H]⁺; | (500 MHz, DMSO-d₆) ppm 4.60 (s, 2 H), 7.24 (d, J = 9.5 Hz, 1 H), 7.28-7.35 (m, 2 H), 7.44 (d, J = 7.5 Hz, 1 H), 7.65-7.68 (m, 2 H), 9.05 (br. s., 1 H). |
| 5 | 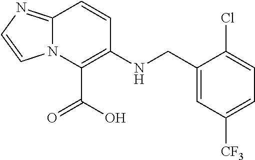 [SMILES: O=C(O)c1c(NCc2cc(C(F)(F)F)ccc2Cl)ccc2nccn12] | (ESI), m/z 370.1 [M + H]⁺; | (500 MHz, DMSO-d₆) ppm 4.71 (s, 2 H), 7.25 (d, J = 9.5 Hz, 1 H), 7.68-7.76 (m, 5 H), 9.09 (br. s., 1 H). |
| 6 | 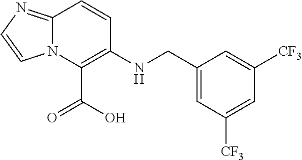 [SMILES: O=C(O)c1c(NCc2cc(C(F)(F)F)cc(C(F)(F)F)c2)ccc2nccn12] | (ESI), m/z 404.1 [M + H]⁺; | (500 MHz, DMSO-d₆) ppm 4.80 (s, 2 H), 7.28 (d, J = 9.5 Hz, 1 H), 7.67 (d, J = 9.5 Hz, 1 H), 7.71 (s, 1 H), 7.99 (s, 1 H), 8.04 (m, 2 H), 9.09 (br. s., 1 H). |
| 7 | 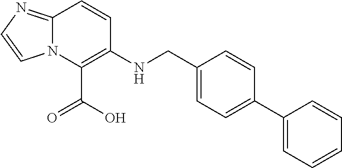 [SMILES: O=C(O)c1c(NCc2ccc(-c3ccccc3)cc2)ccc2nccn12] | Mass spectrum (ESI), m/z 344.1 [M + H]⁺; | (500 MHz, DMSO-d₆) ppm 4.25 (d, J = 5.5 Hz, 2 H), 6.12 (s, 1 H), 6.97 (dd, J = 9.5, 2.0 Hz, 1 H), 7.35- 7.36 (m, 3 H), 7.44-7.50 (m, 4 H), 7.57 (s, 1 H), 7.63- 7.66 (m, 3 H), 7.68 (s., 1 H). |
| 8 | 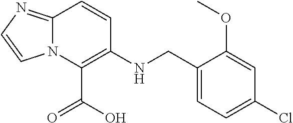 [SMILES: COc1cc(Cl)ccc1CNc1ccc2nccn2c1C(=O)O] | ESI m/z 332.0 [M + 1]⁺ | (400 MHz, DMSO-d₆) ppm 3.85 (s, 3 H), 4.48 (s, 2 H), 6.94 (dd, J = 8.0, 1.6 Hz, 1 H), 7.08 (s, 1 H), 7.23 (d, J = 8.0 Hz, 1 H), 7.42 (d, J = 9.60 Hz, 1 H), 7.72 (d, J = 9.6 Hz, 1 H), 7.80 (s, 1 H), 9.06 (br. s., 1 H). |
| 9 | 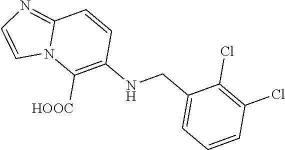 [SMILES: O=C(O)c1c(NCc2cccc(Cl)c2Cl)ccc2nccn12] | ESI m/z 335.9 [M + 1]⁺ | (400 MHz, CD₃OD) ppm: 9.03 (d, J = 2.0 Hz, 1H), 7.96 (d, J = 2.4 Hz, 1H), 7.91 (d, J = 10.0 Hz, 1H), 7.64 (d, J = 10.0 Hz, 1H), 7.52 (d, J = 8.0 Hz, 1H), 7.39(d, J = 7.2 Hz, 1H), 7.30 (t, J = 7.6 Hz, 1H), 4.85 (s, 2H). |
| 10 | 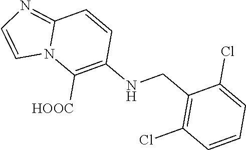 [SMILES: O=C(O)c1c(NCc2c(Cl)cccc2Cl)ccc2nccn12] | ESI m/z 336.0 [M + 1]⁺ | (400 MHz, DMSO-d6) ppm: 8.78 (d, J = 1.2 Hz, 1H), 8.16- 8.08 (m, 2H), 8.03 (d, J = 10.0 Hz, 1H), 7.57 (d, J = 8.0 Hz, 2H), 7.44 (t, J = 8.4 Hz, 1H), 4.84 (s., 2H). |
| 11 | 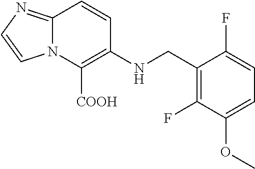 [SMILES: COc1ccc(F)c(CNc2ccc3nccn3c2C(=O)O)c1F] | ESI m/z 334.1 [M + 1]⁺ | (400 MHz, CD₃OD) ppm 9.05 (s, 1H), 8.03-7.84 (m, 3H), 7.13-7.07 (m, 1H), 7.01-6.93 (m, 1H), 4.76 (s, 2H), 3.87 (s, 3H). |
| 12 | 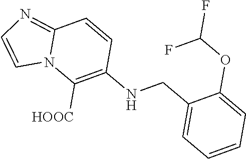 [SMILES: O=C(O)c1c(NCc2ccccc2OC(F)F)ccc2nccn12] | ESI m/z 334.1 [M + 1]⁺ | (400 MHz, DMSO-d6) ppm: 8.89 (s, 1H), 8.04 (s, 1H), 7.91 (d, J = 9.6 Hz, 1H), 7.58 (d, J = 10.0 Hz, 1H), 7.49 (s, 0.3H), 7.42-7.33 (m, 2H), 7.31 (s, 0.5H), 7.28-7.18 (m, 2H), 7.12 (s, 0.3H), 4.71 (s, 2H). |
| 13 | 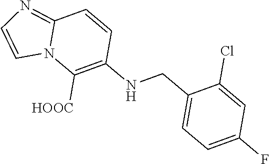 [SMILES: O=C(O)c1c(NCc2ccc(F)cc2Cl)ccc2nccn12] | ESI m/z 320.1 [M + 1]⁺ | (400 MHz, DMSO-d6) ppm 8.86 (d, J = 1.6 Hz, 1H), 8.11 (d, J = 2.0 Hz, 1H), 7.98 (d, J = 10.0 Hz, 1H), 7.62 (d, J = 10.0 Hz, 1H), 7.53 (dd, J = 2.4, 8.8 Hz, 1H), 7.44 (dd, J = 6.0, 8.0 Hz, 1H), 7.23-7.18 (m, 1H), 4.74 (s, 2H). |
| 14 | 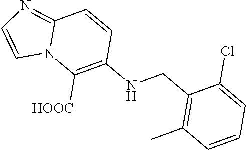 [SMILES: Cc1cccc(Cl)c1CNc1ccc2nccn2c1C(=O)O] | ESI m/z 316.1 [M + 1]⁺ | (400 MHz, DMSO-d6) ppm 8.82 (s, 1H), 8.15-8.08 (m, 2H), 7.99 (d, J = 9.6 Hz, 1H), 7.40 (d, J = 7.6 Hz, 1H), 7.35- 7.25 (m, 2H), 4.72 (s, 2H), 2.43 (s, 3H). |
| 15 | 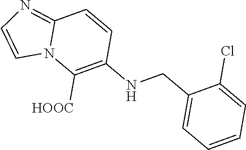 [SMILES: O=C(O)c1c(NCc2ccccc2Cl)ccc2nccn12] | ESI m/z 302.0 [M + 1]⁺ | (400 MHz, DMSO-d6) ppm 8.91 (s, 1H), 8.04 (d, J = 1.6 Hz, 1H), 7.91 (d, J = 10.0 Hz, 1H), 7.55-7.51 (m, 2H), 7.40-7.36 (m, 1H), 7.35- 7.28 (m, 2H), 4.76 (s, 2H). |
| 16 | 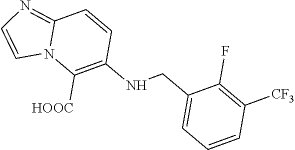 [SMILES: O=C(O)c1c(NCc2cccc(C(F)(F)F)c2F)ccc2nccn12] | ESI m/z 354.2 [M + 1]⁺ | (400 MHz, CD₃OD) ppm 9.02 (d, J = 2.0 Hz, 1H), 7.99- 7.88 (m, 2H), 7.76 (d, J = 10.0 Hz, 1H), 7.74-7.62 (m, 2H), 7.36 (t, J = 7.6 Hz, 1H), 4.88 (s, 2H). |
| 17 | 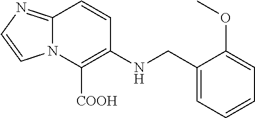 [SMILES: COc1ccccc1CNc1ccc2nccn2c1C(=O)O] | ESI m/z 297.9 [M + 1]⁺ | (400 MHz, CD₃OD) ppm 8.99 (d, J = 2.0 Hz, 1H), 7.94- 7.86 (m, 2H), 7.81 (d, J = 10.0 Hz, 1H), 7.35-7.27 (m, 2H), 7.04 (d, J = 8.4 Hz, 1H), 6.93 (t, J = 7.6 Hz, 1H), 4.67 (s, 2H), 3.91 (s, 3H). |
| 18 | 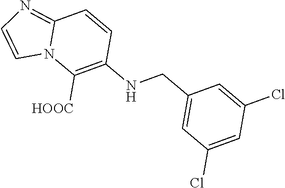 [SMILES: O=C(O)c1c(NCc2cc(Cl)cc(Cl)c2)ccc2nccn12] | ESI m/z 336.1 [M + 1]⁺ | (400 MHz, DMSO-d6) ppm 8.85 (d, J = 1.6 Hz, 1H), 8.10 (d, J = 2.0 Hz, 1H), 7.96 (d, J = 10.0 Hz, 1H), 7.61 (d, J = 10.0 Hz, 1H), 7.52 (s, 1H), 7.43 (s, 2H), 4.74 (s, 2H). |
| 19 | 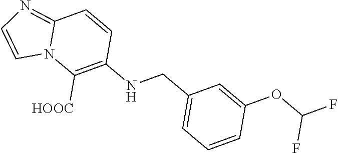 [SMILES: O=C(O)c1c(NCc2cccc(OC(F)F)c2)ccc2nccn12] | ESI m/z 334.1 [M + 1]⁺ | (400 MHz, DMSO-d6) ppm 8.93 (s, 1H), 7.98 (s, 1H), 7.87 (d, J = 9.6 Hz, 1H), 7.52 (d, J = 10.0 Hz, 1H), 7.44- 7.40 (m, 1H), 7.39 (s, 0.3H), 7.24 (s., 1H), 7.22 (s, 0.5H), 7.17 (s., 1H), 7.08 (d, J = 7.6 Hz, 1H), 7.04 (s, 0.2H), 4.70 (s, 2H). |
| 20 | 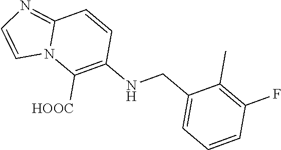 [SMILES: Cc1c(F)cccc1CNc1ccc2nccn2c1C(=O)O] | ESI m/z 300.0 [M + 1]⁺ | (400 MHz, DMSO-d6) ppm 8.85 (s, 1H), 8.11 (s, 1H), 7.97 (d, J = 10.0 Hz, 1H), 7.63 (d, J = 10.0 Hz, 1H), 7.23-7.14 (m, 1H), 7.13- 7.01 (m, 2H), 4.72 (s., 2H), 2.26 (s, 3H). |
| 21 | 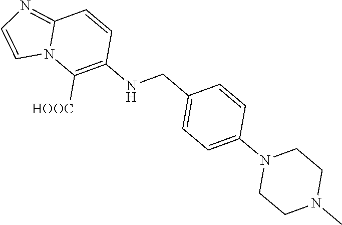 [SMILES: CN1CCN(c2ccc(CNc3ccc4nccn4c3C(=O)O)cc2)CC1] | ESI m/z 366.1 [M + 1]⁺ | (400 MHz, CD₃OD) ppm 9.17 (m, 1H), 7.57 (s, 1H), 7.53-7.51 (d, J = 8.0 Hz, 1H), 7.37-7.35 (d, J = 8.0 Hz, 1H), 7.28-7.26 (d, J = 8.0 Hz, 2H), 6.93-6.91 (d, J = 8.0 Hz, 2H), 4.44 (s, 2H), 3.25(m, 4H), 2.97-2.95 (m, 4H), 2.61 (s, 3H). |
| 22 | 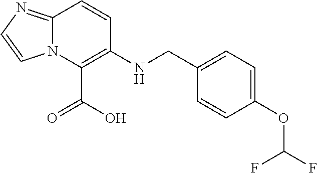 [SMILES: O=C(O)c1c(NCc2ccc(OC(F)F)cc2)ccc2nccn12] | ESI m/z 334.2 [M + 1]⁺ | (400 MHz, DMSO-d6) ppm 8.84 (d, J = 1.6 Hz, 1H), 8.09 (d, J = 2.0 Hz, 1H), 7.96 (d, J = 10.0 Hz, 1H), 7.66 (d, J = 10.0 Hz, 1H), 7.42 (d, J = 8.4 Hz, 2H), 7.40 (s, 0.3H), 7.22 (s, 0.5H), 7.17 (d, J = 8.0 Hz, 2H), 7.03 (s, 0.2H), 4.71 (s, 2H). |
| 23 | 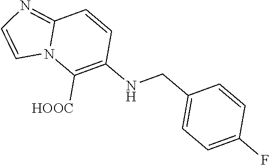 [SMILES: O=C(O)c1c(NCc2ccc(F)cc2)ccc2nccn12] | ESI m/z 286.0 [M + 1]⁺ | (400 MHz, DMSO-d6) ppm 8.83 (s, 1H), 8.09 (s, 1H), 7.97 (d, J = 10.0 Hz, 1H), 7.67 (d, J = 10.0 Hz, 1H), 7.43-7.39 (m, 2H), 7.25- 7.11 (m, 2H), 4.69 (s., 2H). |
| 24 | 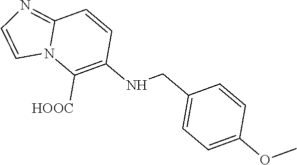 [SMILES: COc1ccc(CNc2ccc3nccn3c2C(=O)O)cc1] | ESI m/z 298.2 [M + 1]⁺ | (400 MHz, DMSO-d6) ppm 8.83 (d, J = 1.6 Hz, 1H), 8.09 (d, J = 1.6 Hz, 1H), 7.97 (d, J = 10.0 Hz, 1H), 7.70 (d, J = 10.0 Hz, 1H), 7.30 (d, J = 8.4 Hz, 2H), 6.92 (d, J = 8.4 Hz, 2H), 4.61 (s, 2H), 3.73 (s, 3H). |
| 25 | 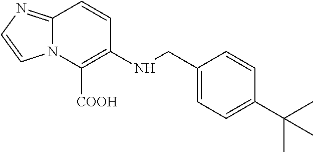 [SMILES: CC(C)(C)c1ccc(CNc2ccc3nccn3c2C(=O)O)cc1] | ESI m/z 324.0 [M + 1]⁺ | (400 MHz, DMSO-d6) ppm 8.82 (d, J = 2.0 Hz, 1H), 8.08 (d, J = 2.0 Hz, 1H), 7.98 (d, J = 10.0 Hz, 1H), 7.70 (d, J = 10.4 Hz, 1H), 7.36 (d, J = 8.0 Hz, 2H), 7.31-7.25 (m, 2H), 4.65 (s., 2H), 1.24 (s, 9H). |
| 26 | 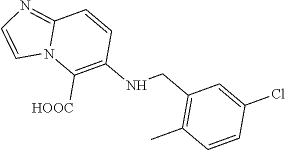 [SMILES: Cc1ccc(Cl)cc1CNc1ccc2nccn2c1C(=O)O] | ESI m/z 316.0 [M + 1]⁺ | (400 MHz, DMSO-d6) ppm 8.85 (s, 1H), 8.10 (d, J = 1.2 Hz, 1H), 7.98 (d, J = 10.0 Hz, 1H), 7.61 (d, J = 10.4 Hz, 1H), 7.31-7.14 (m, 3H), 4.67 (s, 2H), 2.33 (s, 3H). |
| 27 | 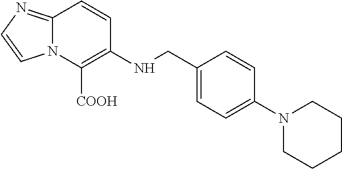 [SMILES: O=C(O)c1c(NCc2ccc(N3CCCCC3)cc2)ccc2nccn12] | ESI m/z 351.1 [M + 1]⁺ | (400 MHz, CD₃OD) ppm 8.98 (s, 1H), 7.47-7.36 (m, 2H), 7.28 (d, J = 8.4 Hz, 2H), 7.14 (d, J = 9.6 Hz, 1H), 6.96 (d, J = 8.4 Hz, 2H), 4.43 (s, 2H), 3.16-3.04 (m, 4H), 1.75-1.69 (m, 4H), 1.63- 1.54 (m, 2H). |
| 28 | 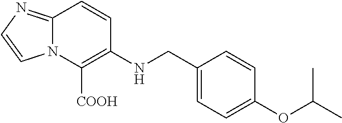 [SMILES: CC(C)Oc1ccc(CNc2ccc3nccn3c2C(=O)O)cc1] | ESI m/z 326.0 [M + 1]⁺ | (400 MHz, CD₃OD) 9.25-9.24 (d, J = 4.0 Hz, 1H), 7.65-7.64 (d, J = 4.0 Hz, 1H), 7.59-7.57 (d, J = 8.0 Hz, 1H), 7.47-7.45 (d, J = 4.0 Hz, 1H), 7.29-7.26 (d, J = 12.0 Hz, 2H), 6.86-6.83 (d, J = 28.0 Hz, 2H), 4.57- |
| | | | 4.51 (m, 1H), 4.47 (s, 2H), |
| | | | 1.28-1.27 (d, J = 4.0 Hz, |
| | | | 6H). |
| 29 | 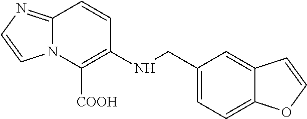 [SMILES: O=C(O)c1c(NCc2ccc3occc3c2)ccc2nccn12] | ESI m/z 308.0 [M + 1]⁺ | (400 MHz, DMSO-d6) ppm 8.85 (d, J = 2.0 Hz, 1H), 8.09 (d, J = 2.0 Hz, 1H), 8.00 (d, J = 2.0 Hz, 1H), 7.95 (d, J = 10.4 Hz, 1H), 7.71 (d, J = 10.0 Hz, 1H), 7.65 (s, 1H), 7.59 (d, J = 8.4 Hz, 1H), 7.34 (d, J = 8.4 Hz, 1H), 6.94 |
| | | | (d, J = 1.6 Hz, 1H), 4.80 (s, |
| | | | 2H). |
| 30 | 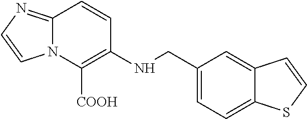 [SMILES: O=C(O)c1c(NCc2ccc3sccc3c2)ccc2nccn12] | ESI m/z 324.0 [M + 1]⁺ | (400 MHz, CD₃OD) ppm 9.01 (d, J = 2.0 Hz, 1H), 7.96- 7.90 (m, 2H), 7.87 (d, J = 10.8 Hz, 2H), 7.76 (d, J = 10.0 Hz, 1H), 7.60 (d, J = 5.6 Hz, 1H), 7.41 (d, J = 7.2 Hz, 1H), 7.36 (d, J = 5.6 Hz, 1H), 4.85 (s, 2H). |
| 31 | 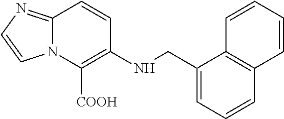 [SMILES: O=C(O)c1c(NCc2cccc3ccccc23)ccc2nccn12] | ESI m/z 318.0 [M + 1]⁺ | (400 MHz, DMSO-d6) ppm 8.85 (d, J = 2.0 Hz, 1H), 8.17 (d, J = 8.4 Hz, 1H), 8.11 (d, J = 2.0 Hz, 1H), 8.02-7.95 (m, 2H), 7.92-7.86 (m, 1H), 7.74 (d, J = 10.0 Hz, 1H), 7.66-7.55 (m, 2H), 7.46 (d, J = 5.2 Hz, 2H), 5.18 |
| | | | (s, 2H). |
| 32 | 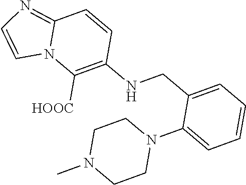 [SMILES: CN1CCN(c2ccccc2CNc2ccc3nccn3c2C(=O)O)CC1] | ESI m/z 365.9 [M + 1]⁺ | (400 MHz, CD₃OD) ppm 8.97 (s, 1H), 7.44 (d, J = 7.6 Hz, 1H), 7.41-7.34 (m, 2H), 7.25-7.16 (m, 2H), 7.12-7.03 (m, 2H), 4.56 (s, 2H), 3.03 (t, J = 4.0 Hz, 4H), 2.72 (br. s., 4H), 2.40 (s, 3H). |
| 33 | 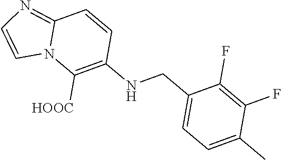 [SMILES: Cc1ccc(CNc2ccc3nccn3c2C(=O)O)c(F)c1F] | ESI m/z 318.0 [M + 1]⁺ | (400 MHz, DMSO-d6) ppm 8.83 (d, J = 1.6 Hz, 1H), 8.10 (d, J = 2.0 Hz, 1H), 7.99 (d, J = 10.0 Hz, 1H), 7.70 (d, J = 10.0 Hz, 1H), 7.11-7.01 (m, 2H), 4.77 (s., 2H), 2.25 (s, 3H). |
| 34 | 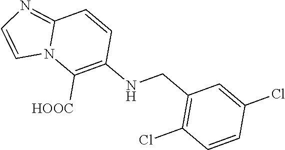 [SMILES: O=C(O)c1c(NCc2cc(Cl)ccc2Cl)ccc2nccn12] | ESI m/z 335.9 [M + 1]⁺ | (400 MHz, DMSO-d6) ppm 8.87 (d, J = 2.0 Hz, 1H), 8.12 (d, J = 2.0 Hz, 1H), 7.98 (d, J = 10.0 Hz, 1H), 7.58 (dd, J = 10.4, 12.8 Hz, 2H), 7.46- 7.39 (m, 2H), 4.76 (s, 2H). |
| 35 | 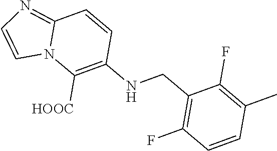 [SMILES: Cc1ccc(F)c(CNc2ccc3nccn3c2C(=O)O)c1F] | ESI m/z 318.0 [M + 1]⁺ | (400 MHz, DMSO-d6) ppm 8.79 (d, J = 1.6 Hz, 1H), 8.10 (dd, J = 3.6, 5.6 Hz, 2H), 7.86 (d, J = 10.0 Hz, 1H), 7.37-7.25 (m, 1H), 7.06 (t, J = 8.8 Hz, 1H), 4.74 (s, 2H), 2.21 (s, 3H). |
| 36 | 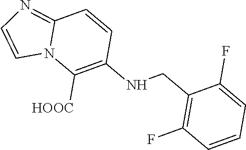 [SMILES: O=C(O)c1c(NCc2c(F)cccc2F)ccc2nccn12] | ESI m/z 304.0 [M + 1]⁺ | (400 MHz, DMSO-d6) ppm 8.80 (d, J = 2.0 Hz, 1H), 8.14- 8.04 (m, 2H), 7.86 (d, J = 10.4 Hz, 1H), 7.49-7.42 (m, 1H), 7.23-7.11 (m, 2H), 4.76 (s, 2H). |
| 37 | 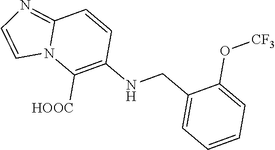 [SMILES: O=C(O)c1c(NCc2ccccc2OC(F)(F)F)ccc2nccn12] | ESI m/z 351.9 [M + 1]⁺ | (400 MHz, CD₃OD) ppm 9.02 (d, J = 2.0 Hz, 1H), 8.01- 7.85 (m, 2H), 7.68 (d, J = 10.0 Hz, 1H), 7.53 (d, J = 7.2 Hz, 1H), 7.48-7.33 (m, 3H), 4.80 (s, 2H). |
| 38 | 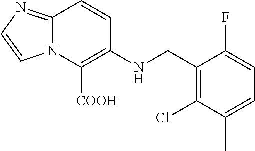 [SMILES: Cc1ccc(F)c(CNc2ccc3nccn3c2C(=O)O)c1Cl] | ESI m/z 334.1 [M + 1]⁺ | (400 MHz, CD₃OD) ppm 8.95 (d, J = 2.0 Hz, 1H), 8.01 (s, 2H), 7.94 (d, J = 2.0 Hz, 1H), 7.41-7.30 (m, 1H), 7.10 (t, J = 9.2 Hz, 1H), 4.85 (s, 2H), 2.39 (s, 3H). |
| 39 | 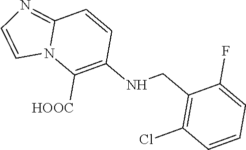 [SMILES: O=C(O)c1c(NCc2c(F)cccc2Cl)ccc2nccn12] | ESI m/z 320.2 [M + 1]⁺ | (400 MHz, DMSO-d6) ppm 8.80 (d, J = 2.0 Hz, 1H), 8.10 (dd, J = 4.0, 6.0 Hz, 2H), 7.91 (d, J = 10.0 Hz, 1H), 7.50-7.40 (m, 2H), 7.37- 7.28 (m, 1H), 4.79 (s, 2H). |
| 40 | 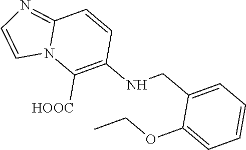 [SMILES: CCOc1ccccc1CNc1ccc2nccn2c1C(=O)O] | ESI m/z 312.0 [M + 1]⁺ | (400 MHz, CD₃OD) ppm 8.98 (d, J = 2.4 Hz, 1H), 7.94- 7.80 (m, 3H), 7.33 (d, J = 7.2 Hz, 1H), 7.31-7.24 (m, 1H), 7.01 (d, J = 8.4 Hz, 1H), 6.91 (t, J = 7.6 Hz, 1H), 4.67 (s, 2H), 4.17-4.12 (m, 2H), 1.48 (t, J = 6.8 Hz, 3H). |
| 41 | 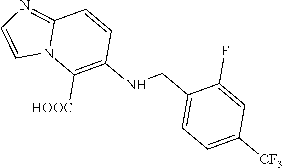 [SMILES: O=C(O)c1c(NCc2ccc(C(F)(F)F)cc2F)ccc2nccn12] | ESI m/z 353.9 [M + 1]⁺ | (400 MHz, DMSO-d6) ppm 8.86 (d, J = 1.6 Hz, 1H), 8.10 (d, J = 2.0 Hz, 1H), 7.96 (d, J = 9.6 Hz, 1H), 7.73 (d, J = 10.4 Hz, 1H), 7.67 (d, J = 10.0 Hz, 1H), 7.55 (s, 2H), 4.87 (s, 2H). |
| 42 | 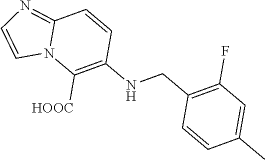 [SMILES: Cc1ccc(CNc2ccc3nccn3c2C(=O)O)c(F)c1] | ESI m/z 300.1 [M + 1]⁺ | (400 MHz, CD₃OD) ppm 8.99 (d, J = 2.0 Hz, 1H), 7.97- 7.89 (m, 2H), 7.78 (d, J = 10.0 Hz, 1H), 7.30 (t, J = 8.0 Hz, 1H), 7.04-6.96 (m, 2H), 4.72 (s, 2H), 2.35 (s, 3H). |
| 43 | 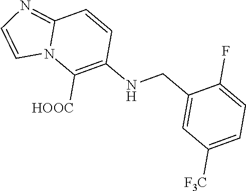 [SMILES: O=C(O)c1c(NCc2cc(C(F)(F)F)ccc2F)ccc2nccn12] | ESI m/z 354.1 [M + 1]⁺ | (400 MHz, CD₃OD) ppm 9.09 (d, J = 2.0 Hz, 1H), 7.96- 7.86 (m, 2H), 7.81-7.65 (m, 3H), 7.39 (t, J = 9.2 Hz, 1H), 4.84 (s, 2H). |
| 44 | 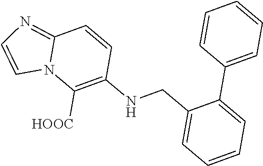 [SMILES: O=C(O)c1c(NCc2ccccc2-c2ccccc2)ccc2nccn12] | ESI m/z 344.0 [M + 1]⁺ | (400 MHz, CD₃OD) ppm 8.96 (d, J = 2.0 Hz, 1H), 7.91 (d, J = 2.0 Hz, 1H), 7.80 (d, J = 10.0 Hz, 1H), 7.54-7.48 (m, 1H), 7.48-7.26 (m, 9H), 4.62 (s, 2H). |
| 45 | 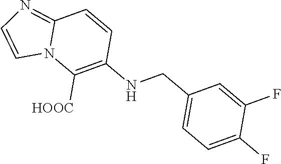 [SMILES: O=C(O)c1c(NCc2ccc(F)c(F)c2)ccc2nccn12] | ESI m/z 304.1 [M + 1]⁺ | (400 MHz, DMSO-d6) ppm 8.84 (d, J = 2.0 Hz, 1H), 8.09 (d, J = 2.0 Hz, 1H), 7.96 (d, J = 9.6 Hz, 1H), 7.64 (d, J = 10.0 Hz, 1H), 7.48-7.35 (m, 2H), 7.22 (s., 1H), 4.70 (s., 2H). |
| 46 | 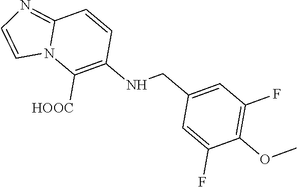 [SMILES: COc1c(F)cc(CNc2ccc3nccn3c2C(=O)O)cc1F] | ESI m/z 333.9 [M + 1]⁺ | (400 MHz, DMSO-d6) ppm 8.84 (d, J = 2.0 Hz, 1H), 8.09 (d, J = 2.0 Hz, 1H), 7.97 (d, J = 10.0 Hz, 1H), 7.62 (d, J = 10.0 Hz, 1H), 7.15 (d, J = 8.8 Hz, 2H), 4.67 (s, 2H), 3.87 (s, 3H). |
| 47 | 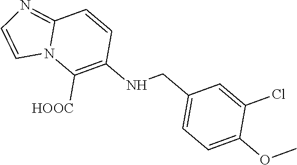 [SMILES: COc1ccc(CNc2ccc3nccn3c2C(=O)O)cc1Cl] | ESI m/z 332.1 [M + 1]⁺ | (400 MHz, CD₃OD) ppm 9.01 (d, J = 2.0 Hz, 1H), 7.94 (d, J = 2.0 Hz, 1H), 7.88 (d, J = 10.0 Hz, 1H), 7.71 (d, J = 10.0 Hz, 1H), 7.42 (d, J = 2.0 Hz, 1H), 7.32 (d, J = 8.4 Hz, 1H), 7.08 (d, J = 8.0 Hz, 1H), 4.65 (s, 2H), 3.88 (s, 3H). |
| 48 | 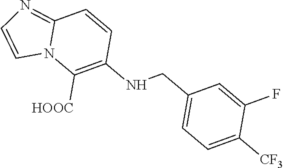 [SMILES: O=C(O)c1c(NCc2ccc(C(F)(F)F)c(F)c2)ccc2nccn12] | ESI m/z 354.0 [M + 1]⁺ | (400 MHz, CD₃OD) ppm 9.04 (d, J = 2.0 Hz, 1H), 7.95 (d, J = 2.4 Hz, 1H), 7.88 (d, J = 10.0 Hz, 1H), 7.70 (t, J = 8.0 Hz, 1H), 7.63 (d, J = 10.0 Hz, 1H), 7.45-7.34 (m, 2H), 4.85 (s, 2H). |
| 49 | 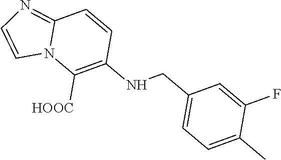 [SMILES: Cc1ccc(CNc2ccc3nccn3c2C(=O)O)cc1F] | ESI m/z 300.1 [M + 1]⁺ | (400 MHz, CD₃OD) ppm 9.01 (s, 1H), 7.94 (s, 1H), 7.89 (d, J = 10.0 Hz, 1H), 7.69 (d, J = 10.0 Hz, 1H), 7.24 (t, J = 7.6 Hz, 1H), 7.17- 7.02 (m, 2H), 4.69 (s, 2H), 2.25 (s, 3H). |
| 50 | 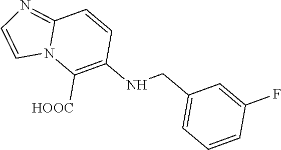 [SMILES: O=C(O)c1c(NCc2cccc(F)c2)ccc2nccn12] | ESI m/z 286.1 [M + 1]⁺ | (400 MHz, CD₃OD) ppm 9.02 (s, 1H), 7.95 (s, 1H), 7.88 (d, J = 9.6 Hz, 1H), 7.69 (d, J = 9.6 Hz, 1H), 7.45- 7.35 (m, 1H), 7.24 (d, J = 8.0 Hz, 1H), 7.15 (d, J = 9.6 Hz, 1H), 7.04 (t, J = 8.4 Hz, 1H), 4.75 (s, 2H). |
| 51 | 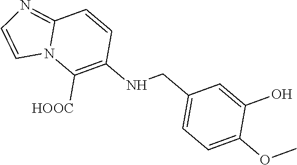 [SMILES: COc1ccc(CNc2ccc3nccn3c2C(=O)O)cc1O] | ESI m/z 314.1 [M + 1]⁺ | (400 MHz, CD₃OD) ppm 9.01 (s, 1H), 7.93 (s, 1H), 7.86 (d, J = 10.0 Hz, 1H), 7.72 (d, J = 9.6 Hz, 1H), 6.95- 6.90 (m, 1H), 6.89-6.79 (m, 2H), 4.59 (s, 2H), 3.85 (s, 3H). |
| 52 | 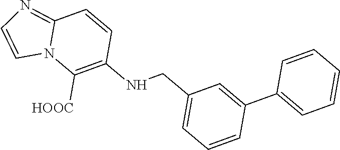 [SMILES: O=C(O)c1c(NCc2cccc(-c3ccccc3)c2)ccc2nccn12] | ESI m/z 344.1 [M + 1]⁺ | (400 MHz, CD₃OD) ppm 9.00 (d, J = 2.0 Hz, 1H), 7.93 (d, J = 2.0 Hz, 1H), 7.89 (d, J = 10.0 Hz, 1H), 7.77 (d, J = 10.0 Hz, 1H), 7.67 (s, 1H), 7.61 (d, J = 7.2 Hz, 2H), 7.56 (d, J = 7.6 Hz, 1H), 7.51- 7.29 (m, 5H), 4.79 (s, 2H). |
| 53 | 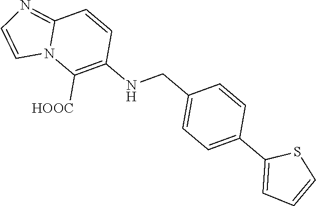 [SMILES: O=C(O)c1c(NCc2ccc(-c3cccs3)cc2)ccc2nccn12] | ESI m/z 350.0 [M + 1]⁺ | (400 MHz, CD₃OD) ppm 9.01 (d, J = 2.0 Hz, 1H), 7.93 (d, J = 1.6 Hz, 1H), 7.89 (d, J = 10.4 Hz, 1H), 7.73 (d, J = 10.0 Hz, 1H), 7.65 (d, J = 8.0 Hz, 2H), 7.43 (d, J = 8.0 Hz, 2H), 7.40-7.36 (m, 2H), 7.13-7.05 (m, 1H), 4.74 (s, 2H). |
| 54 | 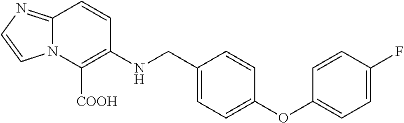 [SMILES: O=C(O)c1c(NCc2ccc(Oc3ccc(F)cc3)cc2)ccc2nccn12] | ESI m/z 378.1 [M + 1]⁺ | (400 MHz, CD₃OD) ppm 9.00 (d, J = 2.0 Hz, 1H), 7.94 (d, J = 2.0 Hz, 1H), 7.90 (d, J = 10.0 Hz, 1H), 7.75 (d, J = 10.4 Hz, 1H), 7.40 (d, J = 8.8 Hz, 2H), 7.14-7.07 (m, 2H), 7.05-6.93 (m, 4H), 4.69 (s, 2H). |
| 55 | 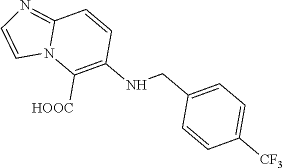 [SMILES: O=C(O)c1c(NCc2ccc(C(F)(F)F)cc2)ccc2nccn12] | ESI m/z 335.9 [M + 1]⁺ | (400 MHz, CD₃OD) ppm 9.03 (d, J = 1.6 Hz, 1H), 7.95 (d, J = 2.0 Hz, 1H), 7.87 (d, J = 10.0 Hz, 1H), 7.72-7.64 (m, 3H), 7.64-7.56 (m, 2H), 4.84 (s, 2H). |
| 56 | 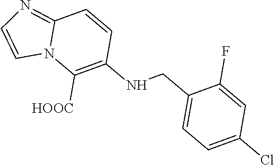 [SMILES: O=C(O)c1c(NCc2ccc(Cl)cc2F)ccc2nccn12] | ESI m/z 320.0 [M + 1]⁺ | (400 MHz, CD₃OD) ppm 9.01 (d, J = 2.0 Hz, 1H), 7.99- 7.88 (m, 2H), 7.74 (d, J = 10.0 Hz, 1H), 7.42 (t, J = 8.4 Hz, 1H), 7.29 (dd, J = 1.6, 10.0 Hz, 1H), 7.22 (d, J = 8.0 Hz, 1H), 4.77 (s, 2H). |
| 57 | 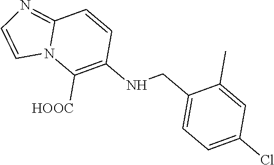 [SMILES: Cc1cc(Cl)ccc1CNc1ccc2nccn2c1C(=O)O] | ESI m/z 316.0 [M + 1]⁺ | (400 MHz, CD₃OD) ppm 9.02 (d, J = 2.4 Hz, 1H), 7.95 (d, J = 2.0 Hz, 1H), 7.90 (d, J = 10.0 Hz, 1H), 7.68 (d, J = 10.0 Hz, 1H), 7.27 (d, J = 8.8 Hz, 2H), 7.22-7.14 (m, 1H), 4.68 (s, 2H), 2.42 (s, 3H). |
| 58 | 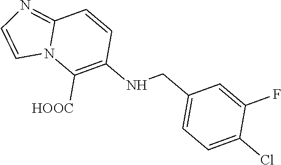 [SMILES: O=C(O)c1c(NCc2ccc(Cl)c(F)c2)ccc2nccn12] | ESI m/z 320.0 [M + 1]⁺ | (400 MHz, CD₃OD) ppm 9.02 (d, J = 1.6 Hz, 1H), 7.95 (d, J = 2.4 Hz, 1H), 7.89 (d, J = 10.0 Hz, 1H), 7.66 (d, J = 10.0 Hz, 1H), 7.48 (t, J = 8.0 Hz, 1H), 7.30 (d, J = 10.0 Hz, 1H), 7.24 (d, J = 8.0 Hz, 1H), 4.75 (s, 2H). |
| 59 | 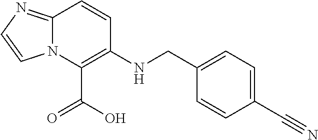 [SMILES: N#Cc1ccc(CNc2ccc3nccn3c2C(=O)O)cc1] | ESI m/z 293.1 [M + 1]⁺ | (400 MHz, D₂O) ppm 8.41 (s, 1H), 7.52 (d, J = 7.6 Hz, 2H), 7.40-7.29 (m, 4H), 7.00 (d, J = 9.6 Hz, 1H), 4.44 (s, 2H). |
| 60 | 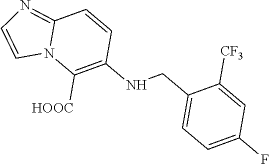 [SMILES: O=C(O)c1c(NCc2ccc(F)cc2C(F)(F)F)ccc2nccn12] | ESI m/z 353.9 [M + 1]⁺ | (400 MHz, DMSO-d6) ppm 8.89 (s, 1H), 8.13 (d, J = 1.6 Hz, 1H), 7.99 (d, J = 9.6 Hz, 1H), 7.78-7.69 (m, 1H), 7.64-7.41 (m, 3H), 4.86 (s., 2H). |
| 61 | 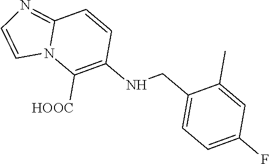 [SMILES: Cc1cc(F)ccc1CNc1ccc2nccn2c1C(=O)O] | ESI m/z 300.1 [M + 1]⁺ | (400 MHz, CD₃OD) ppm 9.01 (s, 1H), 8.01-7.84 (m, 2H), 7.73 (d, J = 10.0 Hz, 1H), 7.36-7.27 (m, 1H), 7.03 (d, J = 8.8 Hz, 1H), 6.92 (t, J = 8.4 Hz, 1H), 4.66 (s, 2H), 2.43 (s, 3H). |
| 62 | 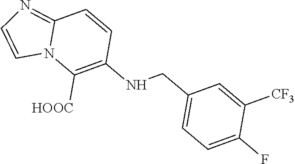 [SMILES: O=C(O)c1c(NCc2ccc(F)c(C(F)(F)F)c2)ccc2nccn12] | ESI m/z 354.0 [M + 1]⁺ | (400 MHz, CD₃OD) ppm 9.02 (s, 1H), 7.95 (s, 1H), 7.89 (d, J = 10.0 Hz, 1H), 7.80-7.64 (m, 3H), 7.36 (t, J = 9.6 Hz, 1H), 4.79 (s, 2H). |
| 63 | 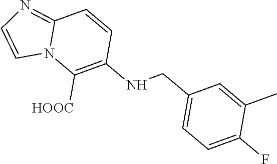 [SMILES: Cc1cc(CNc2ccc3nccn3c2C(=O)O)ccc1F] | ESI m/z 300.0 [M + 1]⁺ | (400 MHz, CD₃OD) ppm 9.01 (d, J = 2.0 Hz, 1H), 7.94 (d, J = 2.0 Hz, 1H), 7.88 (d, J = 10.0 Hz, 1H), 7.71 (d, J = 10.0 Hz, 1H), 7.29 (d, J = 7.6 Hz, 1H), 7.25-7.21 (m, 1H), 7.03 (t, J = 8.8 Hz, 1H), 4.66 (s, 2H), 2.27 (d, J = 0.8 Hz, 3H). |
| 64 | 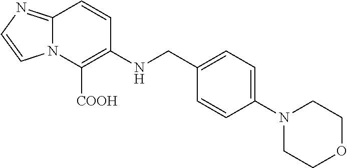 [SMILES: O=C(O)c1c(NCc2ccc(N3CCOCC3)cc2)ccc2nccn12] | ESI m/z 352.9 [M + 1]⁺ | (400 MHz, CD₃OD) ppm 9.02 (d, J = 2.0 Hz, 1H), 7.96 (d, J = 2.0 Hz, 1H), 7.90 (d, J = 10.0 Hz, 1H), 7.84 (d, J = 8.4 Hz, 2H), 7.69 (t, J = 10.0 Hz, 3H), 4.84 (s, 2H), 4.22-4.13 (m, 4H), 3.80-3.71 (m, 4H). |
| 65 | 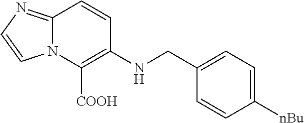 | ESI m/z 324.0 [M + 1]⁺ | (400 MHz, CD₃OD) ppm 9.01 (d, J = 2.0 Hz, 1H), 7.93 (d, J = 2.0 Hz, 1H), 7.86 (d, J = 10.0 Hz, 1H), 7.73 (d, J = 10.0 Hz, 1H), 7.31 (d, J = 8.4 Hz, 2H), 7.20 (d, J = 8.0 Hz, 2H), 4.68 (s, 2H), 2.61 (t, J = 7.6 Hz, 2H), 1.63- |
| | | | 1.56 (m, 2H), 1.42-1.31 |
| | | | (m, 2H), 0.95 (t, J = 7.6 Hz, |
| | | | 3H). |
| 66 | 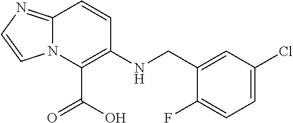 [SMILES: O=C(O)c1c(NCc2cc(Cl)ccc2F)ccc2nccn12] | ESI m/z 319.9 [M + 1]⁺ | (400 MHz, DMSO-d6) ppm 8.85 (d, J = 1.6 Hz, 1H), 8.10 (d, J = 2.0 Hz, 1H), 8.00 (d, J = 10.0 Hz, 1H), 7.70 (d, J = 10.0 Hz, 1H), 7.47-7.37 (m, 2H), 7.36-7.27 (m, 1H), 4.77 (s, 2H). |
| 67 | 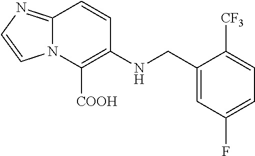 [SMILES: O=C(O)c1c(NCc2cc(F)ccc2C(F)(F)F)ccc2nccn12] | ESI m/z 353.9 [M + 1]⁺ | (400 MHz, DMSO-d6) ppm 8.88 (d, J = 1.2 Hz, 1H), 8.13 (d, J = 1.6 Hz, 1H), 8.00 (d, J = 10.0 Hz, 1H), 7.91-7.88 (m, 1H), 7.45 (d, J = 10.0 Hz, 1H), 7.41-7.29 (m, 2H), 4.89 (s, 2H). |
| 68 | 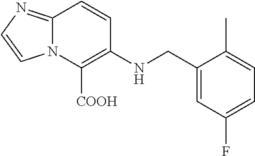 [SMILES: Cc1ccc(F)cc1CNc1ccc2nccn2c1C(=O)O] | ESI m/z 300.0 [M + 1]⁺ | (400 MHz, CD₃OD) ppm 9.03 (d, J = 2.0 Hz, 1H), 7.95 (d, J = 2.4 Hz, 1H), 7.90 (d, J = 10.0 Hz, 1H), 7.65 (d, J = 10.0 Hz, 1H), 7.28-7.24 (m, 1H), 7.02 (dd, J = 2.4, 9.6 Hz, 1H), 6.97-6.92 (m, 1H), 4.69 (s, 2H), 2.39 (s, 3H). |
| 69 | 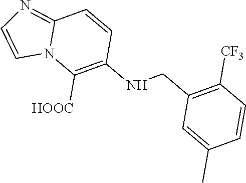 [SMILES: Cc1ccc(C(F)(F)F)c(CNc2ccc3nccn3c2C(=O)O)c1] | ESI m/z 350.1 [M + 1]⁺ | (400 MHz, DMSO-d6) ppm 8.87 (d, J = 1.2 Hz, 1H), 8.12 (d, J = 2.0 Hz, 1H), 8.01 (d, J = 10.0 Hz, 1H), 7.68 (d, J = 7.6 Hz, 1H), 7.51 (d, J = 10.0 Hz, 1H), 7.40-7.26 (m, 2H), 4.82 (s., 2H), 2.32 (s, 3H). |
| 70 | 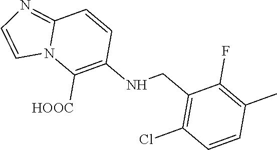 [SMILES: Cc1ccc(Cl)c(CNc2ccc3nccn3c2C(=O)O)c1F] | ESI m/z 334.0 [M + 1]⁺ | (400 MHz, CD₃OD) ppm 8.96 (d, J = 2.0 Hz, 1H), 8.01 (s, 2H), 7.94 (d, J = 2.0 Hz, 1H), 7.30-7.20 (m, 2H), 4.84 (s, 2H), 2.29 (d, J = 2.4 Hz, 3H). |
| 71 | 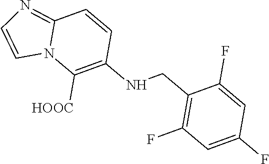 [SMILES: O=C(O)c1c(NCc2c(F)cc(F)cc2F)ccc2nccn12] | ESI m/z 321.9 [M + 1]⁺ | (400 MHz, DMSO-d6) ppm 8.80 (s, 1H), 8.15-8.04 (m, 2H), 7.84 (d, J = 10.0 Hz, 1H), 7.28 (t, J = 8.8 Hz, 2H), 4.73 (s, 2H). |
| 72 | 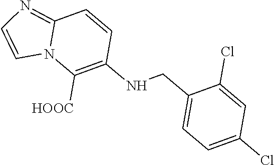 [SMILES: O=C(O)c1c(NCc2ccc(Cl)cc2Cl)ccc2nccn12] | ESI m/z 335.9 [M + 1]⁺ | (400 MHz, CD₃OD) ppm 9.00 (s, 1H), 7.53-7.37 (m, 4H), 7.28 (dd, J = 2.0, 8.8 Hz, 1H), 6.99 (d, J = 10.0 Hz, 1H), 4.61 (s, 2H). |
| 73 | 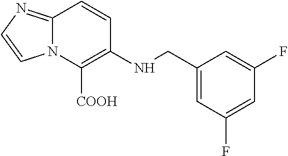 [SMILES: O=C(O)c1c(NCc2cc(F)cc(F)c2)ccc2nccn12] | ESI m/z 304.0 [M + 1]⁺ | (400 MHz, CD₃OD) ppm 9.02 (d, J = 1.6 Hz, 1H), 7.95 (d, J = 2.0 Hz, 1H), 7.91 (d, J = 9.6 Hz, 1H), 7.66 (d, J = 10.0 Hz, 1H), 7.03 (d, J = 6.4 Hz, 2H), 6.83-6.93 (m, 1H), 4.77 (s, 2H). |
| 74 | 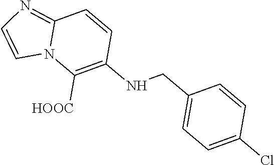 [SMILES: O=C(O)c1c(NCc2ccc(Cl)cc2)ccc2nccn12] | ESI m/z 302.0 [M + 1]⁺ | (400 MHz, CD₃OD) ppm 9.01 (s, 1H), 7.94 (s, 1H), 7.89 (d, J = 10.0 Hz, 1H), 7.69 (d, J = 10.0 Hz, 1H), 7.44-7.32 (m, 4H), 4.72 (s, 1H). |
| 75 | 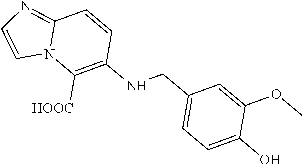 [SMILES: COc1cc(CNc2ccc3nccn3c2C(=O)O)ccc1O] | ESI m/z 314.0 [M + 1]⁺ | (400 MHz, CD₃OD) ppm 8.99 (d, J = 1.6 Hz, 1H), 7.96- 7.87 (m, 2H), 7.77 (d, J = 10.0 Hz, 1H), 7.00 (s, 1H), 6.85(d, J = 8.4 Hz, 1H), 6.79 (d, J = 8.0 Hz, 1H), 4.60 (s, 2H), 3.86 (s, 3H). |
| 76 | 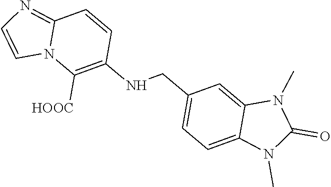 [SMILES: Cn1c(=O)n(C)c2cc(CNc3ccc4nccn4c3C(=O)O)ccc21] | ESI m/z 352.0 [M + 1]⁺ | (400 MHz, CD₃OD) ppm 9.00 (s, 1H), 7.93 (s, 1H), 7.88 (d, J = 10.0 Hz, 1H), 7.78 (d, J = 10.0 Hz, 1H), 7.26-7.18 (m, 2H), 7.14(d, J = 8.4 Hz, 1H), 4.76 (s, 2H), 3.42 (s, 6H). |
| 77 | 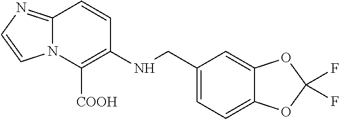 [SMILES: O=C(O)c1c(NCc2ccc3c(c2)OC(F)(F)O3)ccc2nccn12] | ESI m/z 347.9 [M + 1]⁺ | (400 MHz, CD₃OD) 9.01 (s, 1H), 7.45-7.43 (m, 2H), 7.25 (s, 1H), 7.21-7.19 (d, J = 8.0 Hz, 1H), 7.14- 7.11 (m, 2H), 4.54 (s, 2H). |
| 78 | 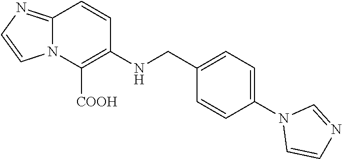 [SMILES: O=C(O)c1c(NCc2ccc(-n3ccnc3)cc2)ccc2nccn12] | ESI m/z 334.0 [M + 1]⁺ | (400 MHz, CD₃OD) ppm 9.51 (s, 1H), 9.02 (d, J = 2.0 Hz, 1H), 8.10 (s, 1H), 7.96 (d, J = 2.0 Hz, 1H), 7.92 (d, J = 10.0 Hz, 1H), 7.81-7.69 (m, 6H), 4.87 (s, 2H). |
| 79 | 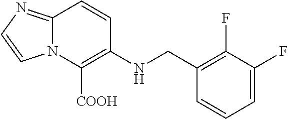 [SMILES: O=C(O)c1c(NCc2cccc(F)c2F)ccc2nccn12] | ESI m/z 304.0 [M + 1]⁺ | (400 MHz, CD₃OD) ppm 9.01 (d, J = 1.6 Hz, 1H), 8.00- 7.87 (m, 2H), 7.77 (d, J = 10.0 Hz, 1H), 7.29-7.11 (m, 3H), 4.84 (s, 2H). |
| 80 | 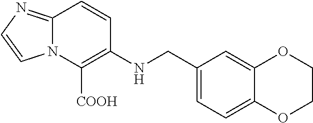 [SMILES: O=C(O)c1c(NCc2ccc3c(c2)OCCO3)ccc2nccn12] | ESI m/z 326.0 [M + 1]⁺ | (400 MHz, CD₃OD) ppm 9.00 (d, J = 1.6 Hz, 1H), 7.93 (d, J = 2.0 Hz, 1H), 7.88 (d, J = 10.0 Hz, 1H), 7.72 (d, J = 10.0 Hz, 1H), 6.90-6.79 (m, 3H), 4.59 (s, 2H), 4.23 (s, 4H). |
| 81 | 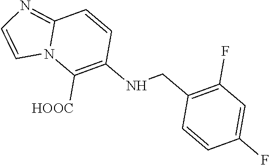 [SMILES: O=C(O)c1c(NCc2ccc(F)cc2F)ccc2nccn12] | ESI m/z 303.9 [M + 1]⁺ | (400 MHz, CD₃OD) ppm 9.01 (s, 1H), 7.93 (d, J = 11.6 Hz, 2H), 7.78 (d, J = 10.0 Hz, 1H), 7.51-7.42 (m, 1H), 7.09-7.01 (m, 1H), 6.98 (t, J = 8.0 Hz, 1H), 4.75 (s, 2H). |
| 82 | 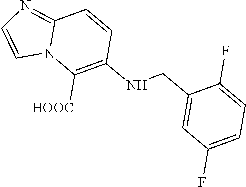 [SMILES: O=C(O)c1c(NCc2cc(F)ccc2F)ccc2nccn12] | ESI m/z 304.0 [M + 1]⁺ | (400 MHz, CD₃OD) ppm 9.01 (s, 1H), 8.00-7.88 (m, 2H), 7.74 (d, J = 10.0 Hz, 1H), 7.24-7.14 (m, 2H), 7.12-7.05 (m, 1H), 4.78 (s, 2H). |
| 83 | 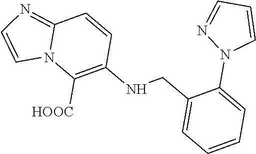 [SMILES: O=C(O)c1c(NCc2ccccc2-n2cccn2)ccc2nccn12] | ESI m/z 334.1 [M + 1]⁺ | (400 MHz, CD₃OD) ppm 8.99 (d, J = 2.0 Hz, 1H), 8.08 (d, J = 2.4 Hz, 1H), 7.93 (d, J = 2.0 Hz, 2H), 7.87 (d, J = 10.0 Hz, 1H), 7.69-7.60 (m, 2H), 7.57-7.46 (m, 3H), 6.65 (t, J = 2.4 Hz, 1H), 4.70 (s, 2H). |
| 84 | 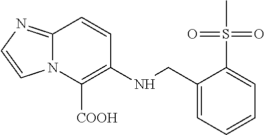 [SMILES: CS(=O)(=O)c1ccccc1CNc1ccc2nccn2c1C(=O)O] | ESI m/z 345.9 [M + 1]⁺ | (400 MHz, CD₃OD) ppm 9.03 (d, J = 1.6 Hz, 1H), 8.11 (d, J = 8.0 Hz, 1H), 7.98- 7.88 (m, 2H), 7.78 (d, J = 10.4 Hz, 1H), 7.74-7.66 (m, 2H), 7.62-7.58 (m, 1H), 5.19 (s, 2H), 3.26 (s, 3H). |
| 85 | 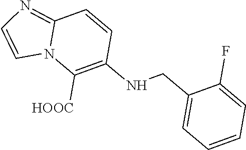 [SMILES: O=C(O)c1c(NCc2ccccc2F)ccc2nccn12] | ESI m/z 286.0 [M + 1]⁺ | (400 MHz, CD₃OD) ppm 9.01 (s, 1H), 7.99-7.87 (m, 2H), 7.78 (d, J = 10.0 Hz, 1H), 7.43 (t, J = 7.6 Hz, 1H), 7.38-7.33 (m, 1H), 7.23- 7.12 (m, 2H), 4.78 (s, 2H). |
| 86 | 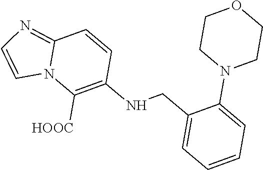 [SMILES: O=C(O)c1c(NCc2ccccc2N2CCOCC2)ccc2nccn12] | ESI m/z 353.0 [M + 1]⁺ | (400 MHz, CD₃OD) ppm 9.00 (s, 1H), 7.97-7.87 (m, 2H), 7.82(d, J = 10.0 Hz 1H), 7.56 (t, J = 8.0 Hz 2H), 7.47 (t, J = 7.6 Hz, 1H), 7.34 (t, J = 7.2 Hz 1H), 4.97 (s., 2H), 4.08 (s., 4H), 3.38 (s., 4H). |
| 87 | 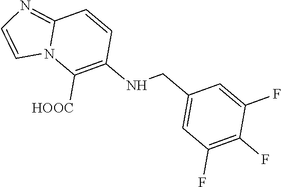 [SMILES: O=C(O)c1c(NCc2cc(F)c(F)c(F)c2)ccc2nccn12] | ESI m/z 322.0 [M + 1]⁺ | (400 MHz, CD₃OD) ppm 9.02 (d, J = 2.0 Hz, 1H), 7.96 (d, J = 2.0 Hz, 1H), 7.91 (d, J = 10.0 Hz, 1H), 7.65 (d, J = 10.0 Hz, 1H), 7.20 (t, J = 7.6 Hz 2H), 4.73 (s, 2H). |
| 88 | 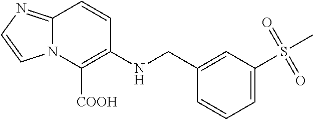 [SMILES: CS(=O)(=O)c1cccc(CNc2ccc3nccn3c2C(=O)O)c1] | ESI m/z 346.0 [M + 1]⁺ | (400 MHz, CD₃OD) ppm 9.03 (s, 1H), 8.00 (s, 1H), 7.95 (s, 1H), 7.90 (d, J = 9.2 Hz, 2H), 7.79 (d, J = 7.6 Hz, 1H), 7.73-7.64 (m, 2H), 4.87 (s, 2H), 3.14 (s, 3H). |
| 89 | 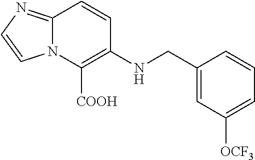 [SMILES: O=C(O)c1c(NCc2cccc(OC(F)(F)F)c2)ccc2nccn12] | ESI m/z 352.0 [M + 1]⁺ | (400 MHz, CD₃OD) ppm 9.01 (d, J = 2.4 Hz, 1H), 7.99- 7.85 (m, 2H), 7.70 (d, J = 13.2 Hz, 1H), 7.53-7.40 (m, 2H), 7.33 (s., 1H), 7.22 (d, J = 10.4 Hz, 1H), 4.79 (s, 2H). |
| 90 | 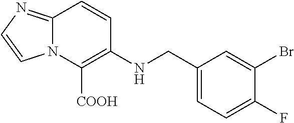 [SMILES: O=C(O)c1c(NCc2ccc(F)c(Br)c2)ccc2nccn12] | ESI m/z 363.9 [M + 1]⁺ | (400 MHz, CD₃OD) ppm 8.99 (s, 1H), 7.67 (d, J = 5.6 Hz, 1H), 7.52-7.32 (m, 3H), 7.16 (t, J = 8.4 Hz, 1H), 7.07 (d, J = 9.6 Hz, 1H), 4.52 (s, 2H). |
| 91 | 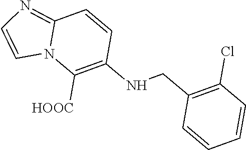 [SMILES: O=C(O)c1c(NCc2ccccc2Cl)ccc2nccn12] | ESI m/z 301.9 [M + 1]⁺ | (400 MHz, CD₃OD) ppm 9.02 (d, J = 2.0 Hz, 1H), 7.94 (d, J = 2.0 Hz, 1H), 7.90 (d, J = 10.0 Hz, 1H), 7.69 (d, J = 10.0 Hz, 1H), 7.43 (s, 1H), 7.40-7.33 (m, 2H), 7.33-7.27 (m, 1H), 4.74 (s, 2H). |
| 92 | 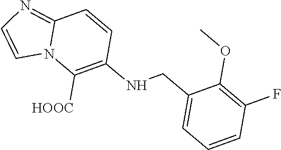 [SMILES: COc1c(F)cccc1CNc1ccc2nccn2c1C(=O)O] | ESI m/z 316.0 [M + 1]⁺ | (400 MHz, CD₃OD) ppm 8.99 (d, J = 1.6 Hz, 1H), 7.99- 7.87 (m, 2H), 7.80 (d, J = 10.0 Hz, 1H), 7.20-7.01 (m, 3H), 4.73 (s, 2H), 4.02 (d, J = 2.0 Hz, 3H). |
| 93 | 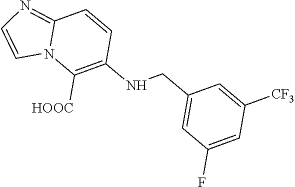 [SMILES: O=C(O)c1c(NCc2cc(F)cc(C(F)(F)F)c2)ccc2nccn12] | ESI m/z 354.0 [M + 1]⁺ | (400 MHz, CD₃OD) ppm 9.08 (d, J = 1.6 Hz, 1H), 7.94 (d, J = 2.0 Hz, 1H), 7.88 (d, J = 10.0 Hz, 1H), 7.66 (d, J = 10.0 Hz, 1H), 7.59 (s, 1H), 7.46 (d, J = 8.8 Hz, 1H), 7.40 (d, J = 8.4 Hz, 1H), 4.83 (s, 2H). |
| 94 | 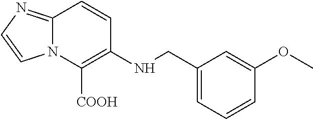 [SMILES: COc1cccc(CNc2ccc3nccn3c2C(=O)O)c1] | ESI m/z 298.0 [M + 1]⁺ | (400 MHz, CD₃OD) ppm 9.01 (d, J = 2.0 Hz, 1H), 7.93 (d, J = 2.0 Hz, 1H), 7.88 (d, J = 10.0 Hz, 1H), 7.72 (d, J = 10.0 Hz, 1H), 7.29 (t, J = 8.0 Hz, 1H), 7.02-6.92 (m, 2H), 6.90-6.82 (m, 1H), 4.69 (s, 2H), 3.79 (s, |
| | | | 3H). |
| 95 | 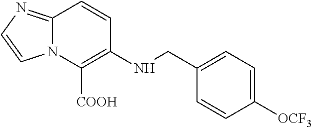 [SMILES: O=C(O)c1c(NCc2ccc(OC(F)(F)F)cc2)ccc2nccn12] | ESI m/z 351.9 [M + 1]⁺ | (400 MHz, CD₃OD) ppm 9.01 (d, J = 2.0 Hz, 1H), 7.95 (d, J = 2.0 Hz, 1H), 7.91 (d, J = 10.0 Hz, 1H), 7.71 (d, J = 10.0 Hz, 1H), 7.52 (d, J = 8.8 Hz, 2H), 7.29 (d, J = 8.0 Hz, 2H), 4.77 (s, 2H). |
| 96 | 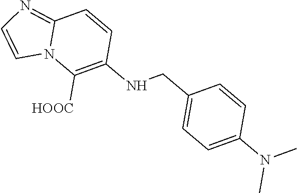 [SMILES: CN(C)c1ccc(CNc2ccc3nccn3c2C(=O)O)cc1] | ESI m/z 311.1 [M + 1]⁺ | (400 MHz, CD₃OD) ppm 8.97 (s, 1H), 7.46-7.37 (m, 2H), 7.25 (d, J = 8.4 Hz, 2H), 7.16 (d, J = 9.6 Hz, 1H), 6.77 (d, J = 8.4 Hz, 2H), 4.40 (s, 2H), 2.90 (s, 6H). |
| 97 | 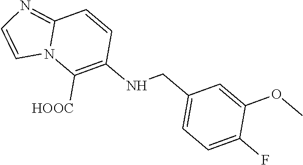 [SMILES: COc1cc(CNc2ccc3nccn3c2C(=O)O)ccc1F] | ESI m/z 316.0 [M + 1]⁺ | (400 MHz, CD₃OD) ppm 9.01 (d, J = 2.0 Hz, 1H), 7.94 (d, J = 2.0 Hz, 1H), 7.90 (d, J = 10.0 Hz, 1H), 7.73 (d, J = 9.6 Hz, 1H), 7.17 (dd, J = 1.6, 8.4 Hz, 1H), 7.10- 7.05 (m, 1H), 6.99-6.92 (m, 1H), 4.69 (s, 2H), 3.88 (s, 3H). |
| 98 | 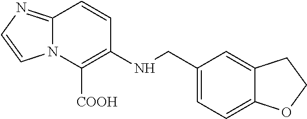 [SMILES: O=C(O)c1c(NCc2ccc3c(c2)CCO3)ccc2nccn12] | ESI m/z 310.1 [M + 1]⁺ | (400 MHz, CD₃OD) ppm 8.97 (s, 1H), 7.47-7.37 (m, 2H), 7.26 (s, 1H), 7.18- 7.09 (m, 2H), 6.67 (d, J = 8.4 Hz, 1H), 4.51 (t, J = 8.4 Hz, 2H), 4.43 (s, 2H), 3.17 (t, J = 8.4 Hz, 2H). |
| 99 | 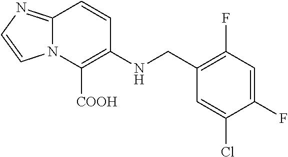 [SMILES: O=C(O)c1c(NCc2cc(Cl)c(F)cc2F)ccc2nccn12] | ESI m/z 337.8 [M + 1]⁺ | (400 MHz, CD₃OD) ppm 9.00 (s, 1H), 7.56 (t, J = 7.6 Hz, 1H), 7.50-7.42 (m, 2H), 7.19-7.09 (m, 2H), 4.56 (s, 2H). |
| 100 | 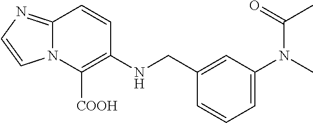 [SMILES: CC(=O)N(C)c1cccc(CNc2ccc3nccn3c2C(=O)O)c1] | ESI m/z 339.0 [M + 1]⁺ | (400 MHz, CD₃OD) ppm 9.01 (d, J = 1.6 Hz, 1H), 7.96- 7.88 (m, 2H), 7.74 (d, J = 10.0 Hz, 1H), 7.53-7.45 (m, 2H), 7.37 (s., 1H), 7.27 (d, J = 7.2 Hz, 1H), 4.78 (s, 2H), 3.26 (s, 3H), 1.85 (s, 3H). |

### Scheme for the Synthesis of Example 101

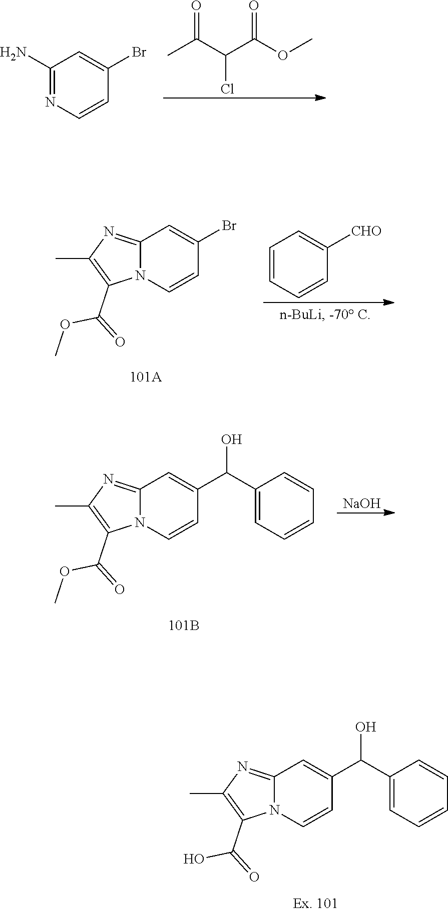

### Example 101A

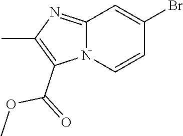

### Example 101A

To a mixture of 4-bromopyridin-2-amine (1.72 g, 9.94 mmol, 1 eq) in MeOH (50 mL) was added methyl 2-chloro-3-oxo-butanoate (478.63 mg, 3.18 mmol, 1.10 eq) dropwise at 20° C. The mixture was stirred at 20° C. for 20 mins, then heated to 70° C. and stirred for 50 hours. The reaction mixture was cooled to 20° C. and filtered. Example 101A (730 mg, 2.71 mmol, 27% yield) was obtained as a white solid.

### Example 101B

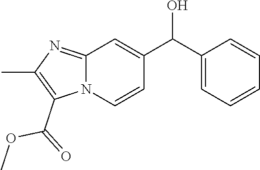 [SMILES: COC(=O)c1c(C)nc2cc(C(O)c3ccccc3)ccn12]

### Example 101B

To a solution of methyl 7-bromo-2-methyl-imidazo[1,2-a]pyridine-3-carboxylate (218 mg, 810 μmol, 1 eq) in THF (10 mL) was added a solution of n-BuLi (2.5 M, 389 μL, 1.2 eq) drop-wise at −70° C. under N₂. The reaction mixture was stirred at −70° C. for 15 mins. Benzaldehyde (103 mg, 972 μmol, 1.2 eq) was added dropwise. The resulting mixture was stirred at 20° C. for 5 hrs. The reaction mixture was quenched with aq. NH₄Cl (5 mL) and extracted with EA (10 mL*2). The combined organic layer was washed with brine (10 mL*2), dried over sodium sulfate, filtered and concentrated. The residue was purified by Prep TLC (EA:PE=1:3). Example 101B (12 mg, 40 μmol, 5% yield) was obtained as a white solid and used directly in next step.

### Example 101

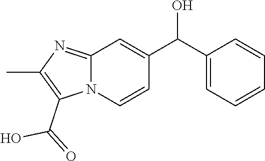 [SMILES: Cc1nc2cc(C(O)c3ccccc3)ccn2c1C(=O)O]

### Example 101

To a solution of methyl 7-[hydroxy(phenyl)methyl]-2-methyl-imidazo[1,2-a]pyridine-3-carboxylate (11 mg, 37 μmol, 1 eq) in THF:MeOH (1:1) (2 mL) was added NaOH (5 M, 15 μL, 2 eq). The mixture was stirred at 20° C. for 10 hrs. Then, the mixture was stirred at 70° C. for 8 hrs. The solvents were removed under reduced pressure then the residue was dissolved in 2 N HCl (5 mL). The resulting mixture was purified by Prep HPLC (YMC-Actus ODS-AQ 150*25 5u, 0.1% TFA-ACN), then treated with 2 N HCl (3 drops) and lyophilized. Example 101 (5.6 mg, 11.8 μmol, 45% yield) was obtained as a white solid.

¹H NMR (400 MHz, METHANOL-d4) δ ppm 2.83 (s, 3H), 5.98 (s, 1H), 7.30-7.51 (m, 7H), 8.04 (s, 1H), 9.52-9.54 (d, J=8.0 Hz 1 H).

### Scheme for the Synthesis of Example 102

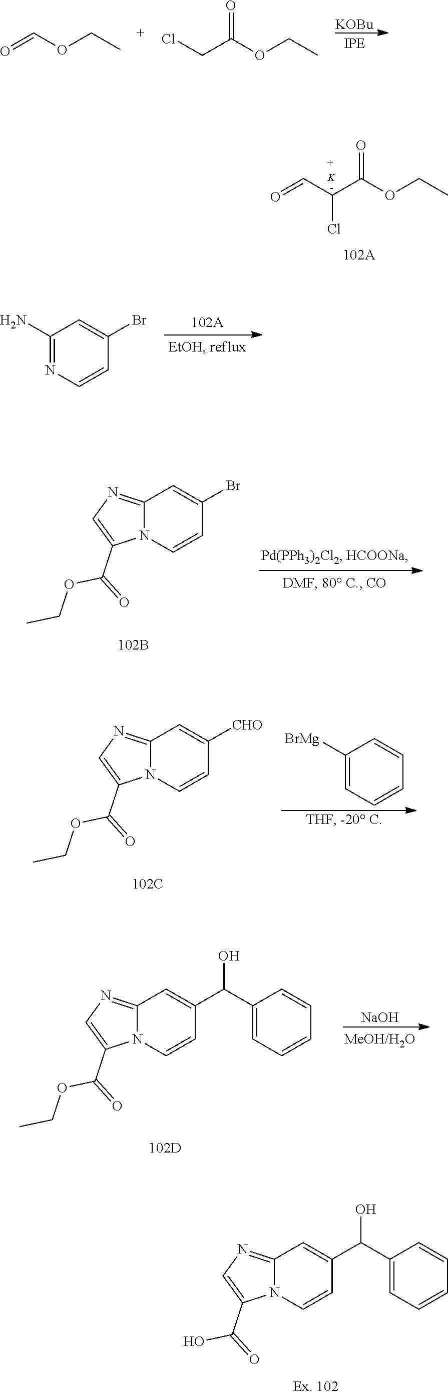

### Example 102A

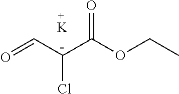

### Example 102A

A mixture of ethyl formate (20.00 g, 163 mmol, 17.39 mL, 1 eq), ethyl chloroacetate (12.09 g, 163 mmol, 13.14 mL, 1 eq), was added to a suspension of potassium tert-butoxide (18.31 g, 163 mmol, 1 eq) in isopropyl ether (200 mL) at 0° C. The reaction was stirred at 25° C. for 12 hr. The resulting precipitates were collected by filtration, and the solid was washed with petroleum ether (100 mL) and dried in vacuo. Example 102A (21.00 g, yield=68%) was obtained as a yellow solid and was used directly in the next step.

¹H NMR: (DMSO-d6, 400 MHz): ppm 8.94 (br. s., 1H), 3.93 (q, J=7.2 Hz, 2H), 1.12 (t, J=7.2 Hz, 3H).

### Example 102B

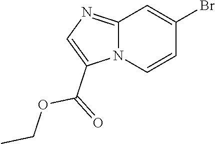

### Example 102B

To a solution of sulfuric acid (4.54 g, 46.24 mmol, 2.46 mL, 1.6 eq) and ethanol (75 mL) at 0° C. was added Example 102A (16.90 g, 89.59 mmol, 3.1 eq) and 2-amino-4-bromopyridine (5.00 g, 28.90 mmol, 1 eq). The reaction was stirred at 80° C. for 5 hr. The reaction was cooled to 25° C. and was diluted with water. The aqueous solution was adjusted to pH=7, and extracted with ethyl acetate (300 mL*3). The combined organic phase was washed with brine (200 mL*2), dried with anhydrous sodium sulfate, filtered and concentrated in vacuo. The residue was purified by silica gel chromatography. Example 102B (1.70 g, 6.32 mmol, yield=22%) was obtained as a white solid.

¹H NMR: (CDCl₃, 400 MHz): ppm 9.19 (d, J=7.2 Hz, 1H), 8.27 (s, 1H), 7.93 (s, 1H), 7.16 (dd, J=1.6, 7.6 Hz, 1H), 4.43 (q, J=7.6 Hz, 2H), 1.43 (t, J=7.2 Hz, 3H).

### Example 102C

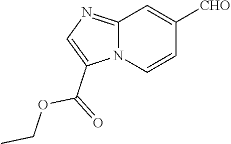 [SMILES: CCOC(=O)c1cnc2cc(C=O)ccn12]

### Example 102C

Toa solution of Example 102B (300 mg, 1.11 mmol, 1 eq) in DMF (10 mL) was added sodium formate (189.6 mg, 2.79 mmol, 2.5 eq), Pd(PPh₃)₂Cl₂ (78.3 mg, 111.5 umol, 0.1 eq). The suspension was degassed and purged with nitrogen three times. The mixture was stirred under carbon monoxide (50 psi) at 80° C. for 12 hr. The reaction was diluted with water (20 mL) and the aqueous solution extracted with ethyl acetate (20 mL*3). The combined organic phases were washed with brine (20 mL*2), dried over anhydrous sodium sulfate, filtered and concentrated in vacuo. The residue was purified by Prep-TLC. Example 102C (130 mg, yield=53.7%) was obtained as a yellow solid.

¹H NMR: (CDCl₃, 400 MHz): ppm 10.08 (s, 1H), 9.40 (d, J=7.2 Hz, 1H), 8.44 (s, 1H), 8.22 (s, 1H), 7.55 (d, J=7.2 Hz, 1H), 4.47 (q, J=7.2 Hz, 2H), 1.45 (t, J=7.2 Hz, 3H).

### Example 102D

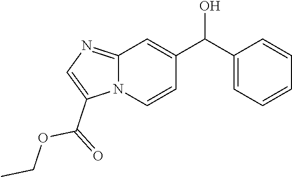 [SMILES: CCOC(=O)c1cnc2cc(C(O)c3ccccc3)ccn12]

### Example 102D

To a solution of Example 102C (100 mg, 458 umol, 1 eq) in THF (2 mL) was added phenyl magnesium bromide (3 M, 183 uL, 1.2 eq) dropwise at −20° C. The reaction was warmed slowly to 0° C. and stirred for 30 minutes (min). The reaction was quenched with water (10 mL) and the aqueous solution was extracted with ethyl acetate (10 mL*3). The combined organic phases were washed with brine (10 mL*2), dried with anhydrous sodium sulfate, filtered and concentrated in vacuo. The crude product Example 102D (80 mg, yield=58.9%) was used directly in the next step. ESI m/z 297.1 [M+1]⁺.

### Example 102

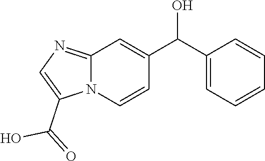 [SMILES: O=C(O)c1cnc2cc(C(O)c3ccccc3)ccn12]

### Example 102

A mixture of Example 102D (80 mg, 270 umol, 1 eq) and sodium hydroxide (21.6 mg, 540 umol, 2 eq) in methanol (5 mL) and water (1 mL) was stirred at 80° C. for 2 hr. The reaction was cooled to 25° C. and concentrated in vacuo. The residue was adjusted to pH=3 with 2N hydrochloric acid aqueous solution. The resulting yellow solid was filtered and dried in vacuo. Example 102 (44.9 mg, yield=60.8%) was obtained as a yellow solid.

¹H NMR: (DMSO-d6, 400 MHz): 9.15 (d, J=7.2 Hz, 1H), 8.19 (s, 1H), 7.77 (s, 1H), 7.44 (d, J=7.2 Hz, 2H), 7.34 (t, J=7.6 Hz, 2H), 7.28-7.21 (m, 1H), 7.14 (dd, J=1.2, 6.8 Hz, 1H), 6.24 (d, J=4.0 Hz, 1H), 5.83 (d, J=2.8 Hz, 1H). ESI m/z 269.1 [M+1]⁺.

### Scheme for the Synthesis of Example 103

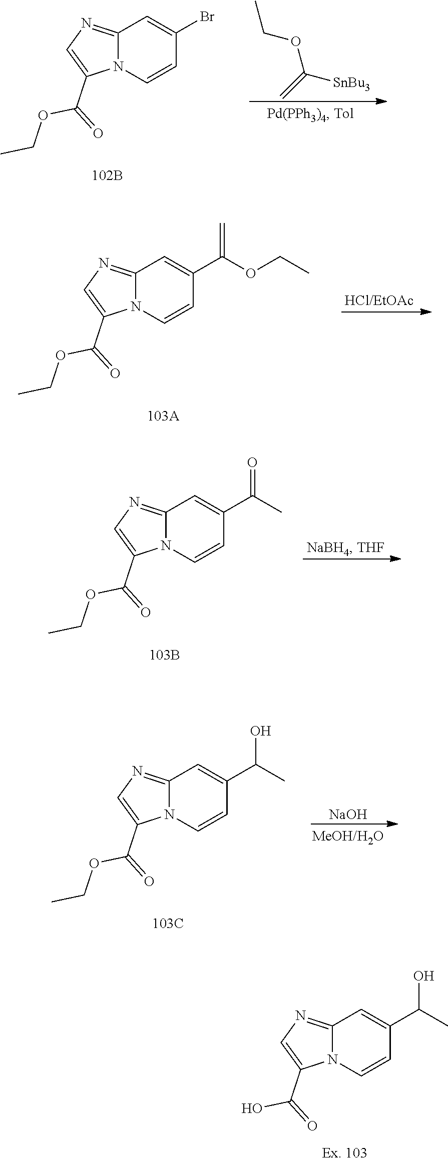

### Example 103A

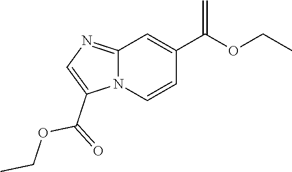 [SMILES: C=C(OCC)c1ccn2c(C(=O)OCC)cnc2c1]

### Example 103A

A mixture of Example 102B (100 mg, 372 umol, 1 eq), tributyl(1-ethoxyvinyl)stannane (161 mg, 446 umol, 151 uL, 1.2 eq) and tetrakis(triphenylphosphine) palladium (43 mg, 37 umol, 0.1 eq) in toluene (2 mL) was degassed and purged with nitrogen for 10 min. The mixture was stirred at 110° C. for 1 hr under a nitrogen atmosphere. The reaction was cooled to 25° C. and diluted with water (10 mL). The aqueous phase was extracted with ethyl acetate (20 mL*3). The combined organic phases were washed with brine (20 mL*2), dried over anhydrous sodium sulfate, filtered and concentrated in vacuo. The residue was purified by PreP-TLC. Example 104A (60 mg, yield=69.5%) was obtained as brown oil. ESI m/z 261.1 [M+1]⁺.

### Example 103B

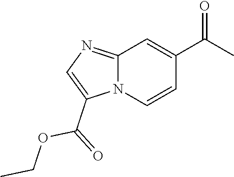 [SMILES: CCOC(=O)c1cnc2cc(C(C)=O)ccn12]

### Example 103B

A solution of Example 103A (60 mg, 231 umol, 1 eq) in HCl/EtOAc (5 mL) was stirred at 25° C. for 30 min. The reaction was concentrated in vacuo. Example 103B (35 mg, yield=65%) was obtained as brown oil. ESI m/z 233.1 [M+1]⁺.

### Example 103C

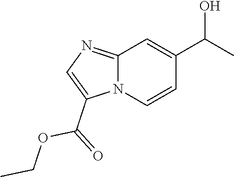 [SMILES: CCOC(=O)c1cnc2cc(C(C)O)ccn12]

### Example 103C

To a solution of Example 103B (35 mg, 151 umol, 1 eq) in THF (5 mL) was added sodium borohydride (11.4 mg, 301 umol, 2 eq) in portions. The reaction was stirred at 25° C. for 30 min. The reaction was diluted with water (10 mL) and the aqueous phase extracted with ethyl acetate (10 mL*3). The combined organic phases were washed with brine (10 mL*2), dried over anhydrous sodium sulfate, filtered and concentrated in vacuo. Example 103C (30 mg, 128 umol, 85% yield) was obtained as brown oil. ESI m/z 235.2 [M+1]⁺.

### Example 103

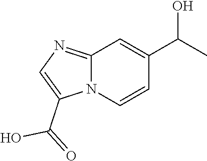

### Example 103

To a solution of Example 103C (30 mg, 128 umol, 1 eq) in ethanol (5 mL) and water (1 mL) was added sodium hydroxide (10.3 mg, 256 umol, 2 eq). The reaction was stirred at 80° C. for 30 min. The reaction was concentrated in vacuo. The residue was adjusted to pH=3 with hydrochloric acid and the aqueous phase purified by prep-HPLC. Example 103 (25 mg, yield=92%) was obtained as a white solid.

¹H NMR: (DMSO-d6, 400 MHz): 9.28 (d, J=6.8 Hz, 1H), 8.44 (s, 1H), 7.76 (s, 1H), 7.37 (s, 1H), 4.90 (d, J=6.4 Hz, 1H), 1.40 (d, J=6.8 Hz, 3H). ESI m/z 207.1[M+1]⁺.

### Scheme for the Synthesis of Example 104

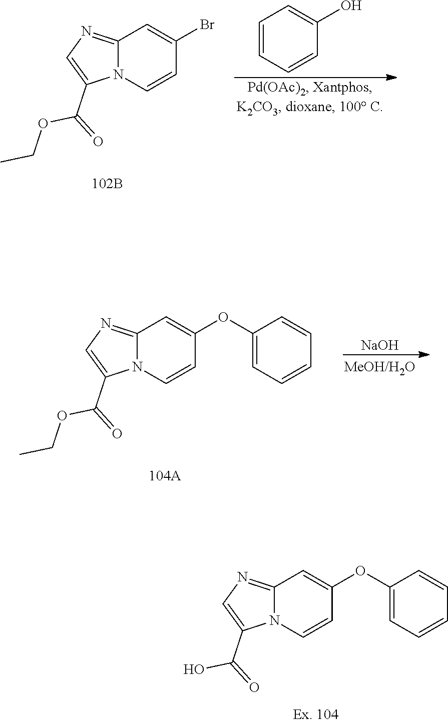

### Example 104A

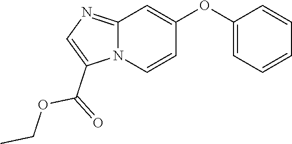 [SMILES: CCOC(=O)c1cnc2cc(Oc3ccccc3)ccn12]

### Example 104A

To a solution of Example 102B (200 mg, 743 umol, 1 eq) in dioxane (2 mL) was added phenol (140 mg, 1.49 mmol, 131 uL, 2 eq), Pd(OAc)₂ (16.7 mg, 74 umol, 0.1 eq), xantphos (86 mg, 149 umol, 0.2 eq) and K₂CO₃ (308 mg, 2.23 mmol, 3 eq). The mixture was stirred at 100° C. for 12 hr. The reaction was cooled to 25° C. and concentrated in vacuo. The residue was diluted with water (20 mL). The aqueous phase was extracted with ethyl acetate (20 mL*3). The combined organic phases were washed with brine (20 mL*2), dried with anhydrous sodium sulfate, filtered and concentrated in vacuo. The residue was purified by Prep-TLC. Example 104A (50 mg, yield=24%) was obtained as a brown solid.

### Example 104

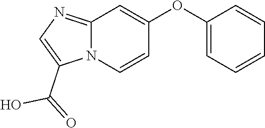 [SMILES: O=C(O)c1cnc2cc(Oc3ccccc3)ccn12]

### Example 104

A mixture of Example 104A (50 mg, 177 umol, 1 eq) and NaOH (14 mg, 354 umol, 2 eq) in MeOH (5 mL) and H₂O (1 mL) was stirred at 80° C. for 2 hr. The mixture was adjusted to pH=3 with 1N HCl. The aqueous solution was purified by Prep-HPLC. Example 105 (31 mg, yield=60%) was obtained as white solid.

¹H NMR: (DMSO-d6, 400 MHz): 9.31 (d, J=8.0 Hz, 1H), 8.40 (s, 1H), 7.55 (t, J=8.0 Hz, 2H), 7.36 (t, J=8.0 Hz, 1H), 7.28-7.23 (m, 3H), 7.04 (d, J=4.0 Hz, 1H).

### Scheme for the Synthesis of Example 105

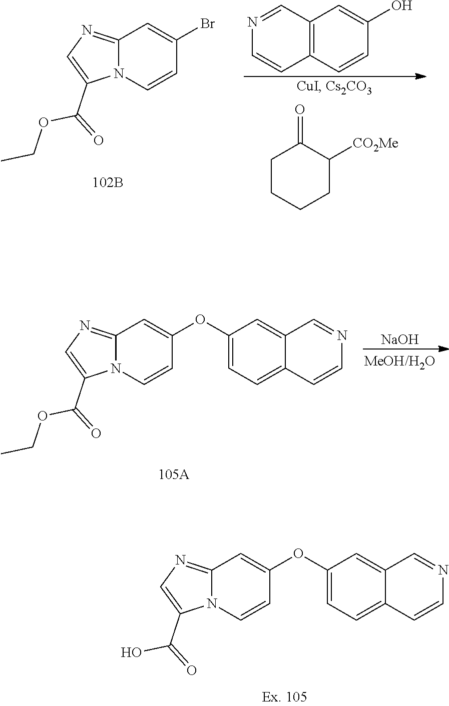

### Example 105A

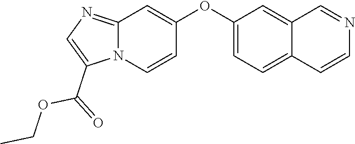 [SMILES: CCOC(=O)c1cnc2cc(Oc3ccc4ccncc4c3)ccn12]

### Example 105A

In a sealed tube, a solution of isoquinolin-7-ol (108 mg, 743 umol, 1 eq), Example 102B (200 mg, 743 umol, 1 eq), methyl 2-oxo cyclohexane carboxylate (23 mg, 149 umol, 21 uL, 0.2 eq), CuI (14 mg, 74 umol, 0.1 eq) and Cs₂CO₃ (484 mg, 1.49 mmol, 2 eq) in DMF (1 mL) was degassed and purged with N₂ for 10 min. The reaction was stirred at 100° C. for 12 hr. The reaction was cooled to 25° C. and diluted with water (10 mL). The aqueous solution was extracted with ethyl acetate (10 mL*3). The combined organic phases were washed with brine (10 mL*2), dried over anhydrous sodium sulfate, filtered and concentrated in vacuo. The residue was purified by Prep-TLC. Example 105A (100 mg crude) was obtained as a brown solid and was used directly in the next step.

### Example 105

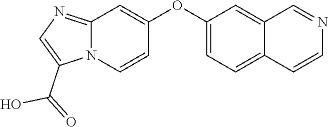 [SMILES: O=C(O)c1cnc2cc(Oc3ccc4ccncc4c3)ccn12]

### Example 105

To a solution of Example 105A (100 mg, 300 umol, 1 eq) in H₂O (1 mL) and MeOH (4 mL) was added NaOH (24 mg, 600 umol, 2 eq). The mixture was stirred at 80° C. for 30 min. The reaction was concentrated in vacuo. The residue was adjusted to pH=3 with HCl (1M) solution and purified by Prep-HPLC. Example 105 (8 mg, yield=9%) was obtained as a white solid.

¹H NMR: (DMSO-d6, 400 MHz): 9.46 (s, 1H), 9.29 (s, 1H), 8.51 (d, J=4.0 Hz, 1H), 8.12 (d, J=8.0 Hz, 1H), 8.05 (s, 1H), 7.89 (d, J=8.0 Hz, 1H), 7.84 (s, 1H), 7.73-7.70 (m, 1H), 7.20 (s, 1H), 7.07 (d, J=4.0 Hz, 1H).

### Scheme for the Synthesis of Example 106

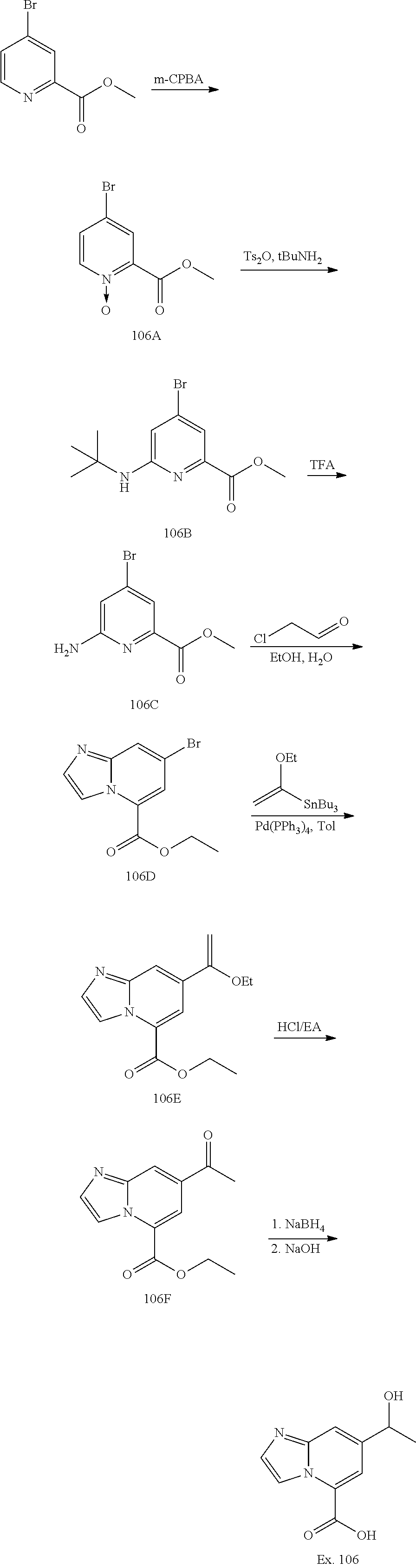

### Example 106A

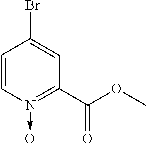

### Example 106A

To a solution of methyl 4-bromopyridine-2-carboxylate (4.00 g, 18.52 mmol, 1 eq) in DCM (100 mL) was added 3-chloroperbenzoic acid (4.57 g, 20.37 mmol, 1.1 eq) in one portion at 0° C. under N₂. The mixture was stirred at 20° C. for 12 hrs. Additional 3-chloroperbenzoic acid (4.57 g, 20.37 mmol, 1.1 eq) was added. The mixture was stirred at 20° C. for another 36 hrs. The reaction mixture was filtered. The filtrate was washed with aq. NaHCO₃ (50 mL), H₂O (2*50 mL), brine (2*50 mL), dried over sodium sulfate, filtered and concentrated. The residue was purified by column chromatography on silica gel (gradient 10 to 100% EA in PE). Example 106A (1.69 g crude) was obtained as a white solid and used directly in next step.

### Example 106B

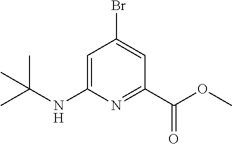 [SMILES: COC(=O)c1cc(Br)cc(NC(C)(C)C)n1]

### Example 106B

To a mixture of Example 106A (1.69 g, 7.28 mmol, 1 eq) and 2-methylpropan-2-amine (4.79 g, 65.52 mmol, 6.84 mL, 9 eq) in CHCl₃ (50 mL) was added p-tolylsulfonyl 4-methylbenzenesulfonate (10.69 g, 32.76 mmol, 4.5 eq) portionwise at 0° C. under N₂. The mixture was stirred at 0° C. for 30 mins. H₂O (50 mL) was added and the aqueous layer was extracted with DCM (2*50 mL). The combined organic layer was washed with H₂O (2*40 mL), brine (2*40 mL), dried over sodium sulfate, filtered and concentrated. The residue was purified by column chromatography on silica gel (EA:PE=1:5). Example 106B (1.1 g, crude) was obtained as a white solid and used directly in next step.

### Example 106C

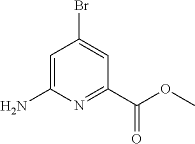

### Example 106C

A mixture of Example 107B (1.31 g, 3.83 mmol, 1 eq) in TFA (10 mL) was heated to 80° C. for 12 hrs. The solvent was concentrated and the residue was dissolved in DCM (100 mL). The resulting mixture was washed with aq. NaHCO₃ (3*30 mL), H₂O (3*30 mL), brine (30 mL), dried over sodium sulfate, filtered and concentrated. The residue was purified by column chromatography on silica gel (EA:PE=1:2). Example 106C (664 mg, crude) was obtained as a brown oil and used directly in next step.

### Example 106D

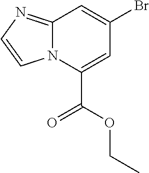

### Example 106D

A mixture of Example 106C (644 mg, 2.79 mmol, 1 eq), 2-chloroacetaldehyde (40% in H2O, 1.63 mL, 23.33 eq) and NaHCO₃ (398 mg, 4.74 mmol, 1.7 eq) in EtOH (10 mL) was heated to 80° C. 12 hours. The mixture was cooled to 20° C. and basified with aq. Na₂CO₃. The aqueous phase was extracted with ethyl acetate (30 mL*2). The combined organic phase was washed with brine (10 mL*2), dried with anhydrous sodium sulfate, filtered and concentrated in vacuo. The residue was purified by column chromatography (gradient 5 to 50% EA in PE). Example 106D (600 mg crude) was obtained as a brown oil and used directly in next step.

### Example 106E

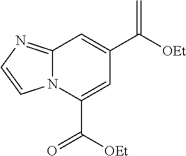 [SMILES: C=C(OCC)c1cc(C(=O)OCC)n2ccnc2c1]

### Example 106E

To a mixture of Example 107D (158 mg, 587 umol, 1 eq) and tributyl(1-ethoxyvinyl)stannane (254 mg, 704 umol, 238 uL, 1.2 eq) in toluene (3 mL) was added tetrakis(triphenylphosphine) palladium (68 mg, 59 umol, 0.1 eq) under N₂. The mixture was heated to 110° C. and stirred for 2 hours. The reaction mixture was filtered and the filtrate was concentrated. The residue was purified by Prep TLC (EA:PE=1:2). Example 106E (45 mg, 173 umol, 29% yield) was obtained as a brown oil.

### Example 106F

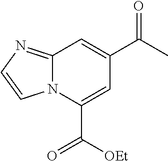 [SMILES: CCOC(=O)c1cc(C(C)=O)cc2nccn12]

### Example 106F

A mixture of Example 107E (45 mg, 173 umol, 1 eq) in HCl/EtOAc (2 mL) was stirred for 20 mins. The solvent was removed in vacuo. Example 106F (41 mg crude) was used directly in the next step.

### Example 106G

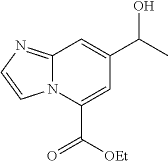 [SMILES: CCOC(=O)c1cc(C(C)O)cc2nccn12]

### Example 106G

To a mixture of Example 106F (41 mg, 177 umol, 1 eq) in MeOH (1 mL) was added NaBH₄ (6.7 mg, 177 umol, 1 eq) in one portion at 5° C. The mixture was stirred at 5° C. for 10 mins. H₂O (0.5 mL) was added and the resulting mixture was concentrated in vacuo. Example 106G (66 mg crude) was used directly in the next step.

### Example 106

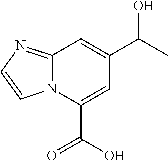

### Example 106

To a mixture of Example 106G (66 mg, 282 umol, 1 eq) in MeOH (2 mL) and H₂O (1 mL) was added NaOH (22.5 mg, 563.5 umol, 2 eq) in one portion at 20° C. The mixture was heated to 70° C. and stirred for 1 hr. The mixture was adjusted to pH 3 with 1 N HCl and concentrated. The residue was purified by Prep HPLC (Welch Ultimate AQ-C18 150*30 mm*5 um, 0.1% TFA-ACN), treated with 1N HCl and lyophilized. Example 106 (14.7 mg, 60 umol, 21.3% yield, HCl) was obtained as a white solid.

¹H NMR (CD₃OD, 400 MHz): 1.54-1.57 (d, J=12.0 Hz, 3H), 5.10-5.14 (q, J=12.0 Hz, 1H), 8.14-8.16 (m, 2H), 8.31 (s, 1H), 9.15 (s, 1H).

### Scheme for the Synthesis of Example 107

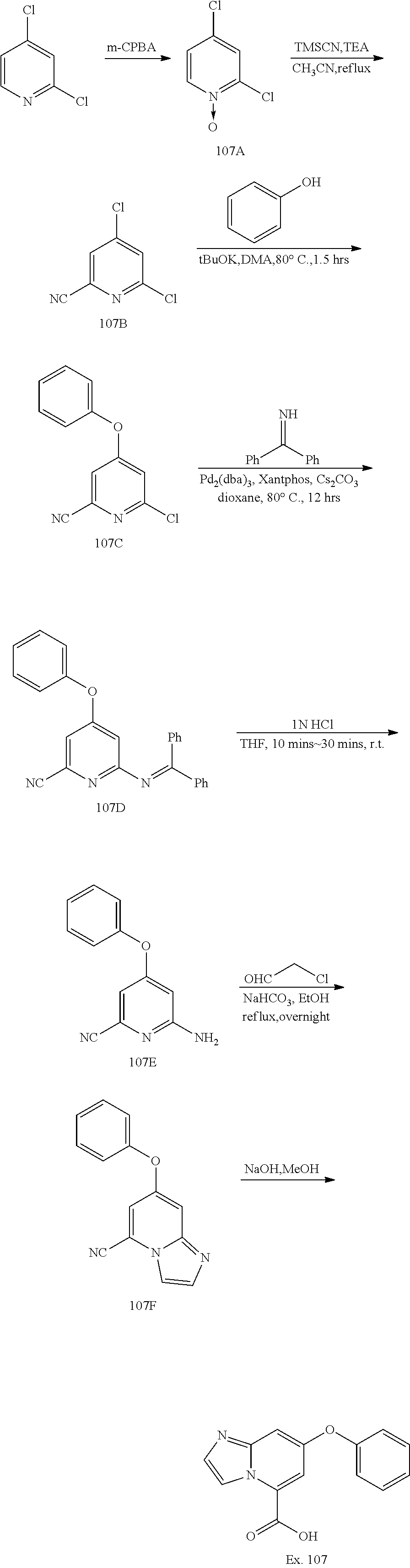

### Example 107A

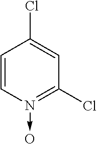

### Example 107A

To a mixture of 2,4-dichloropyridine (9.00 g, 60.81 mmol, 6.57 mL, 1 eq) in DCM (150 mL) was added m-CPBA (16.36 g, 73 mmol, 77% purity, 1.2 eq) in portions at 5° C. The mixture was stirred at 5° C. for 30 mins then stirred for 12 hours at 25° C. The reaction mixture was filtered. The filtrate was washed with aq. NaHCO₃ (100 mL), H₂O (100 mL), brine (100 mL), dried over sodium sulfate, filtered and concentrated. The residue was purified by column chromatography to give Example 107A (5.40 g, 32.9 mmol, 54.2%) as a white solid. ESI m/z 164.0[M+1]⁺.

### Example 107B

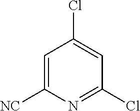

### Example 107B

A mixture of Example 108A (5.40 g, 32.93 mmol, 1 eq) and TEA (6.66 g, 65.86 mmol, 9.12 mL, 2 eq) in CH₃CN (40 mL) was heated at 80° C. for 12 hrs. The mixture was concentrated in vacuo. The residue was washed with H₂O (200 mL*2), brine (200 mL*2), dried over sodium sulfate, filtered and concentrated. The residue was purified by column chromatography to give Example 107B (2.00 g, 11.56 mmol, yield=35%) as a red solid. ESI m/z 172.0[M+1]⁺.

¹H NMR: (CDCl₃, 400 MHz): s ppm 7.67 (s, 1H), 7.62 (s, 1H).

### Example 107C

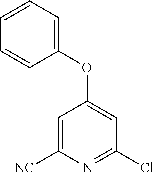 [SMILES: N#Cc1cc(Oc2ccccc2)cc(Cl)n1]

### Example 107C

To a mixture of phenol (65 mg, 694 umol, 61 uL, 1.2 eq) in DMA (2 mL) was added t-BuOK (77.8 mg, 694 umol, 1.2 eq) in one portion at 25° C. under N₂. The mixture was stirred at 25° C. for 30 mins, and Example 108B (100 mg, 578 umol, 1 eq) was added. The reaction was heated to 80° C. and stirred for 4 hours. The mixture was diluted with H₂O (10 mL) and extracted with EtOAc (30 mL). The organic layer was washed with H₂O (10 mL*2), brine (10 mL), dried over sodium sulfate, filtered and concentrated. The residue was purified by prep TLC (EtOAc:PE=1:10). Example 107C (162 mg, yield=73%, 60% purity) was obtained as a colorless oil. ESI m/z 230.0[M+1]⁺.

### Example 107D

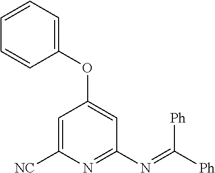 [SMILES: N#Cc1cc(Oc2ccccc2)cc(N=C(c2ccccc2)c2ccccc2)n1]

### Example 107D

Example 107C (162 mg, 421 umol, 1 eq), diphenylmethanimine (92 mg, 506 umol, 1.2 eq), Xantphos (49 mg, 84 umol, 0.2 eq) and Pd₂(dba)₃ (39 mg, 42. umol, 0.1 eq) in dioxane (5 mL) was degassed with N₂ and then heated to 85° C. for 2 hours under N₂. The mixture was filtered and the filtrate was concentrated. The residue was purified by prep-TLC (EtOAc:PE=1:10). Example 107D (100 mg, yield=63%) was obtained as a yellow solid. ESI m/z 375.1[M+1]⁺.

¹H NMR: (CDCl3, 400 MHz): δ ppm 7.53-7.51 (m, 2H), 7.41-7.37 (m, 2H), 7.29-7.26 (m, 6H), 7.15-7.14 (m, 2H), 6.92 (s, 2H), 6.91 (s, 1H), 6.86-6.84 (d, J=8.0 Hz, 2H), 6.13 (s, 1H).

### Example 107E

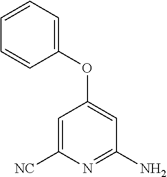 [SMILES: N#Cc1cc(Oc2ccccc2)cc(N)n1]

### Example 107E

To a mixture of Example 107D (100 mg, 266 umol, 1 eq) in THF (2 mL) was added HCl (1 M, 266 uL, 1 eq) in one portion at 25° C. The mixture was stirred at 25° C. for 5 mins. The mixture was adjusted to pH=10 with aq. Na₂CO₃. The resulting mixture was extracted with EtOAc (20 mL*2). The combined organic layer was washed with H₂O (10 mL), brine (10 mL), dried over sodium sulfate, filtered and concentrated. The residue was treated with EtOAc (0.5 mL) and PE (10 mL), and then filtered. Example 107E (41 mg, yield=73%) was obtained as a white solid. ESI m/z 375.1[M+1]⁺.

### Example 107F

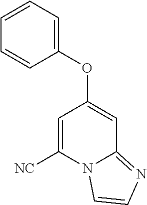 [SMILES: N#Cc1cc(Oc2ccccc2)cc2nccn12]

### Example 107F

To a mixture of Example 107E (41 mg, 194 umol, 1 eq) and 2-chloroacetaldehyde (500 mg, 6.37 mmol, 410 uL, 32.8 eq) in EtOH (5 mL) was added NaHCO₃ (28 mg, 330 umol, 1.7 eq) in one portion at 25° C. under N₂. The mixture was stirred at 80° C. for 12 hrs. The mixture was concentrated in vacuo. The residue was adjusted to pH=10 with aq. Na₂CO₃. The resulting mixture was extracted with DCM (20 mL*2), washed with brine (20 mL), dried over sodium sulfate, filtered and concentrated. The residue was purified by prep-TLC (EtOAc:PE=1:2). Example 107F (40 mg crude) was obtained as a brown oil. ESI m/z 235.1[M+1]⁺.

### Example 107

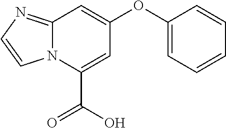 [SMILES: O=C(O)c1cc(Oc2ccccc2)cc2nccn12]

### Example 107

To a mixture of Example 107F (40 mg, 170 umol, 1 eq) in H₂O (1 mL) was added NaOH (6.8 mg, 170 umol, 1 eq) in one portion at 25° C. The mixture was heated to 80° C. and stirred for 12 hours. The mixture was concentrated in vacuo. The residue was adjusted to pH=3 with 1 N HCl and filtered. Example 107 (7.8 mg, 30 umol, yield=18%) was obtained as a white solid. ESI m/z 254.1[M+1]⁺.

¹H NMR: (CD₃OD, 400 MHz): s ppm 9.10 (s, 1H), 7.83 (s, 1H), 7.77 (s, 1H), 7.59-7.55 (m, 2H), 7.42-7.40 (m, 1H), 7.30-7.28 (d, J=8.0 Hz, 2H), 7.07 (s, 1H).

EXAMPLE 108 was prepared in a manner similar to that used for Example 107.

### Example 108A

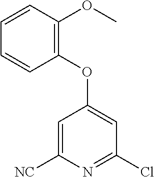 [SMILES: COc1ccccc1Oc1cc(Cl)nc(C#N)c1]

### Example 108A

To a mixture of 2-methoxyphenol (420 mg, 3.38 mmol, 378 uL, 1.17 eq) in DMA (5 mL) was added t-BuOK (389 mg, 3.47 mmol, 1.2 eq) in one portion at 25° C. under N₂. The mixture was stirred at 25° C. for 30 mins, then Example 107B (500 mg, 2.89 mmol, 1 eq) was added, and the mixture heated to 80° C. and stirred for 1.5 hours. The mixture was diluted with H₂O (40 mL) and extracted with EtOAc (40 mL*3). The organic layer was washed with H₂O (40 mL*2), brine (40 mL*2), dried over sodium sulfate, filtered and concentrated. The residue was purified by column chromatography. Example 108A (581 mg, yield=77%) was obtained as a yellow solid. ESI m/z 261.0[M+1]⁺.

### Example 108B

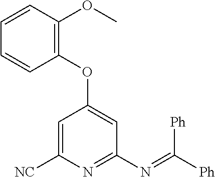 [SMILES: COc1ccccc1Oc1cc(C#N)nc(N=C(c2ccccc2)c2ccccc2)c1]

### Example 108B

Example 108A (581 mg, 2.23 mmol, 1 eq), diphenylmethanimine (485 mg, 2.68 mmol, 1.2 eq), Cs₂CO₃ (2.91 g, 8.92 mmol, 4 eq) and Pd₂(dba)₃ (204 mg, 223 umol, 0.1 eq) in dioxane (20 mL) was de-gassed and then heated to 85° C. for 12 hours under N₂. The mixture was concentrated in vacuo. The mixture was filtered and the filtrate concentrated. The residue was purified by column chromatography. Example 108B (655 mg, yield=72%) was obtained as a yellow solid. ESI m/z 406.1[M+1]⁺.

¹H NMR: (CDCl3, 400 MHz): s ppm 7.76 (s, 2H), 7.40-7.33 (m, 7H), 7.25 (s, 2H), 7.00-6.88 (m, 3H), 6.85 (s, 1H), 6.15 (s, 1H), 3.73 (s, 3H).

### Example 108C

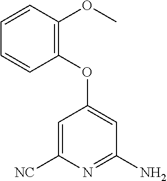 [SMILES: COc1ccccc1Oc1cc(N)nc(C#N)c1]

### Example 108C

To a mixture of Example 108B (655 mg, 1.62 mmol, 1 eq) in THF (5 mL) was added HCl (1 M, 1.62 mL, 1 eq) at 25° C. The mixture was stirred at 25° C. for 10 mins. The mixture was extracted with EtOAc (10 mL). The organic layer was dried over sodium sulfate, filtered and concentrated. The residue was treated with EtOAc (3 mL) and PE (20 mL) and then filtered. Example 108C (336 mg, yield=86%) was obtained as a white solid. ESI m/z 242.1[M+1]⁺.

### Example 108D

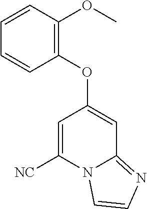 [SMILES: COc1ccccc1Oc1cc(C#N)n2ccnc2c1]

### Example 108D

To a mixture of 2-chloroacetaldehyde (10.64 g, 135.5 mmol, 8.72 mL, 97.5 eq) and Example 108C (336 mg, 1.39 mmol, 1 eq) in EtOH (10 mL) was added NaHCO₃ (164 mg, 1.95 mmol, 1.4 eq) in one portion at 25° C. under N₂. The mixture was heated to 80° C. and stirred for 12 hours. The mixture was concentrated in vacuo. The residue was adjusted to pH=10 with aq. Na₂CO₃. The resulting mixture was extracted with DCM (40 mL*2), washed with brine (20 mL), dried over sodium sulfate, filtered and concentrated. The residue was purified by Prep TLC. Example 108D (389 mg crude) was obtained as a yellow solid and used directly in next step. ESI m/z 266.1[M+1]⁺.

¹H NMR: (CDCl3, 400 MHz): s ppm 7.80 (s, 1H), 7.69 (s, 1H), 7.29-7.28 (m, 2H), 7.15-7.13 (m, 1H), 7.08-7.04 (m, 3H), 3.80 (s, 3H).

### Example 108

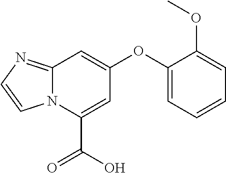 [SMILES: COc1ccccc1Oc1cc(C(=O)O)n2ccnc2c1]

### Example 108

To a mixture of Example 108D (389 mg, 1.47 mmol, 1 eq) in MeOH (50 mL) and H₂O (5 mL) was added NaOH (59 mg, 1.47 mmol, 1 eq) in one portion at 25° C. The mixture was heated to 80° C. and stirred for 12 hours. The mixture was concentrated in vacuo. The residue was adjusted to pH=3 with 1 N HCl and filtered. Example 108 (7.8 mg, yield=2%) was obtained as a white solid. ESI m/z 285.1[M+1]⁺.

¹H NMR: (CD₃OD, 400 MHz): s ppm 9.08 (s, 1H), 7.81 (s, 1H), 7.71 (s, 1H), 7.39-7.37 (m, 1H), 7.29-7.24 (m, 2H), 7.13-7.11 (m, 1H), 6.97 (s, 1H), 3.81 (s, 1H).

EXAMPLE 109 was prepared in a manner similar to that used for Example 107.

### Example 109A

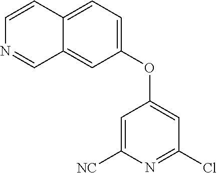 [SMILES: N#Cc1cc(Oc2ccc3ccncc3c2)cc(Cl)n1]

### Example 109A

To a mixture of Example 107B (250 mg, 1.45 mmol, 1 eq) in DMA (6 mL) was added t-BuOK (179 mg, 1.60 mmol, 1.1 eq) in one portion at 25° C. under N₂. The mixture was stirred at 25° C. for 30 mins. Isoquinolin-7-ol (231 mg, 1.59 mmol, 1.1 eq) was added. Then the reaction was heated to 80° C. and stirred for 1 hour. The reaction mixture was filtered. The filtrate was diluted with H₂O (40 mL) and extracted with EtOAc (40 mL*3). The organic layer was washed with H₂O (40 mL*2), brine (40 mL*2), dried over sodium sulfate, filtered and concentrated. The residue was purified by column chromatography. Example 109A (228 mg, yield=56%) was obtained as a colorless oil. ESI m/z 282.0[M+1]⁺.

¹H NMR: (DMSO-d6, 400 MHz): ppm 9.31 (s, 1H), 8.66-8.65 (d, J=4 Hz, 1H), 8.04-8.01 (d, J=12 Hz, 1H), 7.77-7.72 (m, 2H), 7.49-7.46 (m, 1H), 7.27-7.26 (d, J=4 Hz, 1H), 7.07-7.06 (d, J=4 Hz, 1H).

### Example 109B

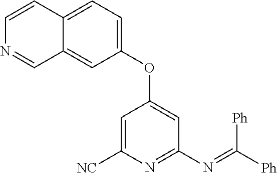 [SMILES: N#Cc1cc(Oc2ccc3ccncc3c2)cc(N=C(c2ccccc2)c2ccccc2)n1]

### Example 109B

Example 109A (228 mg, 809 umol, 1 eq), diphenylmethanimine (161 mg, 890 umol, 149 uL, 1.1 eq), Cs₂CO₃ (1.05 g, 3.24 mmol, 4 eq) and Pd₂(dba)₃ (74 mg, 81 umol, 0.1 eq) in dioxane (10 mL) was degassed and then heated to 85° C. for 12 hours under N₂. The mixture was filtered and the filtrate was concentrated. The residue was purified by column chromatography. Example 109B (243 mg crude) was obtained as a pale yellow solid and used directly in next step. ESI m/z 427.1[M+1]⁺.

### Example 109C

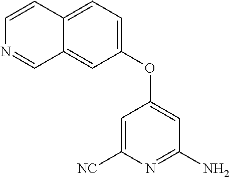 [SMILES: N#Cc1cc(Oc2ccc3ccncc3c2)cc(N)n1]

### Example 109C

To a mixture of Example 109B (228 mg, 535 umol, 1 eq) in THF (3 mL) was added HCl (19.5 mg, 535 umol, 19 uL, 1 eq) at 25° C. The mixture was stirred at 25° C. for 10 mins. The mixture was extracted with EtOAc (10 mL). The organic layer was dried over sodium sulfate, filtered and concentrated. The residue was treated with EtOAc (3 mL) and PE (20 mL), and then filtered. Example 109C (160 mg crude) was obtained as a white solid and used directly in next step. ESI m/z 263.1[M+1]⁺.

### Example 109D

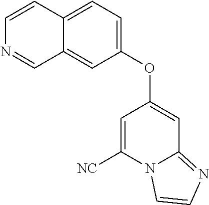 [SMILES: N#Cc1cc(Oc2ccc3ccncc3c2)cc2nccn12]

### Example 109D

To a mixture of Example 109C (160 mg, 610 umol, 1 eq) and 2-chloroacetaldehyde (6.35 g, 81 mmol, 5.20 mL, 133 eq) in EtOH (10 mL) was added NaHCO₃ (87 mg, 1.04 mmol, 1.7 eq) at 25° C. The mixture was heated to 80° C. and stirred for 12 hours. The mixture was concentrated in vacuo. The residue was adjusted to pH=10 with aq. Na₂CO₃. The resulting mixture was extracted with DCM (40 mL*2), washed with brine (20 mL), dried over sodium sulfate, filtered and concentrated. The residue was purified by Prep TLC. Example 109D (157 mg, crude) was obtained as a brown solid and used directly in next step. ESI m/z 287.1[M+1]⁺.

### Example 109

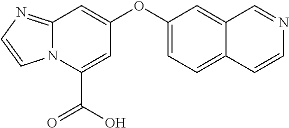 [SMILES: O=C(O)c1cc(Oc2ccc3ccncc3c2)cc2nccn12]

### Example 109

To a mixture of Example 109D (157 mg, 548 umol, 1 eq) in MeOH (10 mL) was added NaOH (1 M, 548 uL, 1 eq). The mixture was heated to 80° C. and stirred for 12 hours. The mixture was concentrated in vacuo. The residue was adjusted to pH=3 with 1 N HCl and filtered. Example 109 (16 mg, yield=8.7%) was obtained as a grey solid. ESI m/z 306.1[M+1]⁺.

¹H NMR: (DMSO-d6, 400 MHz): δ ppm 9.29 (s, 1H), 8.90 (s, 1H), 8.52 (s, 1H), 8.15-8.12 (d, J=12 Hz, 1H), 7.91-7.87 (m, 2H), 7.80 (s, 1H), 7.75-7.73 (d, J=8 Hz, 1H), 7.58 (s, 1H), 7.46 (s, 1H).

### Scheme for the Synthesis of Examples 110 and 111

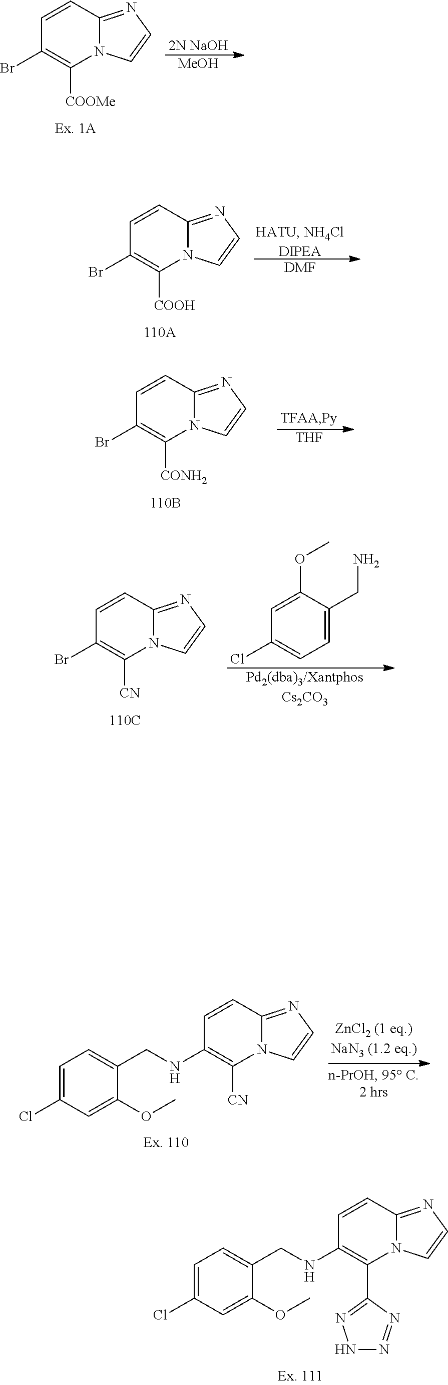

### Example 110A

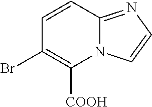 [SMILES: O=C(O)c1c(Br)ccc2nccn12]

### Example 110A

To a mixture of Example 1A (700 mg, 2.74 mmol, 1 eq) in H₂O (1 mL) and MeOH (10 mL) was added NaOH (219 mg, 5.48 mmol, 2 eq). The mixture was stirred at 25° C. for 1 hour. The mixture was concentrated in vacuo. The residue was adjusted to pH 3 with 2 N HCl, and filtered. Example 110A (630 mg crude) was obtained as a grey solid and used directly in next step. ESI m/z 241.0[M+1]⁺.

### Example 110B

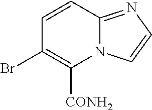 [SMILES: NC(=O)c1c(Br)ccc2nccn12]

### Example 110B

A mixture of Example 110A (389 mg, 1.61 mmol, 1 eq), DIPEA (834 mg, 6.46 mmol, 1.13 mL, 4 eq), NH₄C₁ (604 mg, 11.30 mmol, 395 uL, 7 eq) and HATU (920 mg, 2.42 mmol, 1.5 eq) in DMF (10 mL) was stirred at 25° C. for 12 hrs. The mixture was diluted with EtOAc (100 mL) and washed with H₂O (30 mL*2). The separated organic layer was washed with brine (30 mL*2), dried over sodium sulfate, filtered and concentrated. The residue was treated with EtOAc (10 mL) and filtered. Example 110B (180 mg crude) was collected as a grey solid. ESI m/z 242.1[M+1]⁺.

### Example 110C

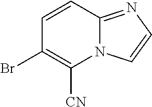 [SMILES: N#Cc1c(Br)ccc2nccn12]

### Example 110C

To a mixture of Example 110B (300 mg, 1.25 mmol, 1 eq) in THF (1 mL) was added pyridine (375 mg, 4.74 mmol, 382 uL, 3.79 eq) followed by TFAA (289 mg, 1.37 mmol, 191 uL, 1.1 eq) dropwise at 0° C. The mixture was stirred at 25° C. for 12 hrs. The mixture was poured into ice water and extracted with EtOAc (50 mL*2). The organic layer was washed with aq. NaHCO₃ (50 mL), brine (50 mL), dried over sodium sulfate, filtered and concentrated. Example 110C (120 mg crude) was obtained as a grey solid and used directly in next step. ESI m/z 222.1[M+1]⁺.

### Example 110

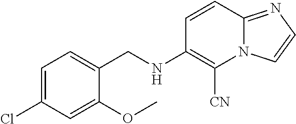 [SMILES: COc1cc(Cl)ccc1CNc1ccc2nccn2c1C#N]

### Example 110

A stirred mixture of Example 110C (100 mg, 450 umol, 1 eq), (4-chloro-2-methoxy-phenyl)methanamine (77 mg, 450 umol, 1 eq), Pd₂(dba)₃ (41 mg, 45 umol, 0.1 eq), Cs₂CO₃ (587 mg, 1.80 mmol, 4 eq) and Xantphos (52 mg, 90 umol, 0.2 eq) in dioxane (1 mL) was degassed and then heated to 100° C. for 10 hours under N₂. The mixture was filtered and concentrated. The residue was purified by prep-TLC (DCM:MeOH=25:1). Example 110 (46 mg, 146 umol, 32% yield) was obtained as a white solid. ESI m/z 313.2[M+1]⁺.

¹H NMR (CDCl₃ 400 MHz): 3.90 (s, 3H), 4.53-4.52 (d, J=4 Hz, 2H), 5.27-5.24 (m, 1 H), 6.95-6.93 (m, 2H), 7.09-7.07 (d, J=8 Hz, 1H), 7.17-7.15 (d, J=8 Hz, 1H), 7.68-7.67 (d, J=4 Hz, 2 H), 8.38-8.36 (m, 1H).

### Example 111

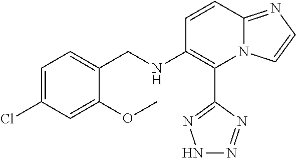 [SMILES: COc1cc(Cl)ccc1CNc1ccc2nccn2c1-c1nn[nH]n1]

### Example 111

A mixture of Example 110 (79 mg, 253 umol, 1 eq), NaN₃ (20 mg, 303 umol, 1.2 eq), and ZnCl₂ (34 mg, 253 umol, 1 eq) in n-PrOH (2 mL) was degassed and then heated to 95° C. for 2 hours under N₂. The mixture was concentrated and 5% NaOH (5 mL) was added. The mixture was filtered and the filtrate was adjusted to pH 3 with 2 N HCl, and then filtered. The residue was treated with DCM:MeOH (20 mL: 1 mL) and filtered. Example 111 (50 mg, 132 umol, 52% yield) was collected as a white solid. ESI m/z 356.2[M+1]⁺.

¹H NMR (CD₃OD 400 MHz): 3.90 (s, 3H), 4.72 (d, 2H), 6.93-6.91 (m, 1H), 7.04 (s, 1 H), 7.33-7.29 (m, 1H), 7.95-7.91 (m, 2H), 8.04 (s, 1H), 9.42 (s, 1H).

### Scheme for the Synthesis of Example 112

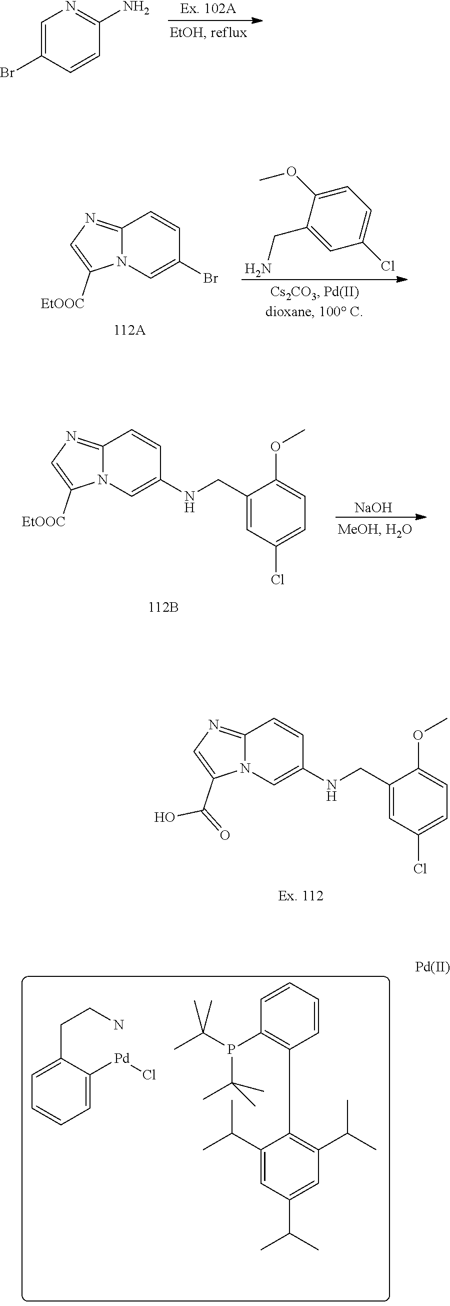

### Example 112A

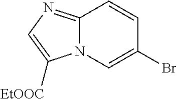 [SMILES: CCOC(=O)c1cnc2ccc(Br)cn12]

### Example 112A

To a solution of H2SO₄ (1.81 g, 18.50 mmol, 986 uL, 1.6 eq) and EtOH (25 mL) at 0° C. was added Example 102A (6.76 g, 35.8 mmol, 3.1 eq) and 2-amino-5-bromopyridine (2.00 g, 11.56 mmol, 1 eq). The reaction was stirred at 80° C. for 5 hr. The reaction was cooled to 25° C. To the mixture was added water and the pH of the solution was adjusted to 7 with saturated NaHCO₃. The mixture was extracted with ethyl acetate (300 mL*3). The combined organic phase was washed with brine (200 mL*2), dried with anhydrous sodium sulfate, filtered and concentrated in vacuo. The residue was purified by silica gel chromatography (Petroleum ether: Ethyl acetate=10/1, 3/1). Example 112A (1.00 g, 3.72 mmol, 32% yield) was obtained as a white solid. ESI m/z 268.9[M+1]⁺.

¹H NMR: (CDCl3, 400 MHz): ppm 9.56 (s, 1H), 8.38 (s, 1H), 8.03 (d, J=8.8 Hz, 1H), 7.6 (dd, J=1.6, 9.6 Hz, 1H), 4.47-4.42 (m, 2H), 1.43 (t, J=7.2 Hz, 3H).

### Example 112B

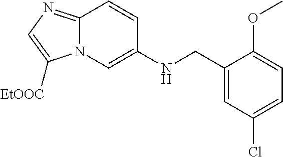 [SMILES: CCOC(=O)c1cnc2ccc(NCc3cc(Cl)ccc3OC)cn12]

### Example 112B

To a solution of Example 112A (100 mg, 372 umol, 1 eq) and 5-chloro-2-methoxybenzyl amine (80 mg, 372 umol, 1 eq) in dioxane (2 mL) was added Cs₂CO₃ (242 mg, 743 umol, 2 eq) and [2-(2-aminoethyl)phenyl]-chloro-palladium; ditert-butyl-[2-(2,4,6-triisopropylphenyl)phenyl]phosphine (25.5 mg, 37 umol, 0.1 eq) under N₂. The reaction was stirred at 100° C. for 12 hours (hr). The reaction was cooled to 25° C. and water (10 mL) was added. The aqueous phase was extracted with ethyl acetate (20 mL*3). The combined organic phase was washed with brine (20 mL*2), dried with anhydrous sodium sulfate, filtered and concentrated in vacuo. The residue was purified by Prep-TLC (Petroleum ether/Ethyl acetate=2/1). Example 112B (45 mg, 125 umol, 34% yield) was obtained as a brown solid. ESI m/z 360.1[M+1]^{P}.

### Example 112

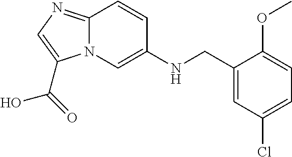 [SMILES: COc1ccc(Cl)cc1CNc1ccc2ncc(C(=O)O)n2c1]

### Example 112

To a solution of Example 112B (35 mg, 97 umol, 1 eq) in MeOH (2 mL) and H₂O (400 uL) was added NaOH (7.8 mg, 195 umol, 2 eq). The reaction was stirred at 80° C. for 1 h. The reaction was cooled to 25° C. and concentrated in vacuo. The residue was adjusted to pH=3 and the aqueous solution purified by Prep-HPLC (YMC-Actus ODS-AQ 150*30 5u, water (0.1% TFA)-ACN). Example 112 (20 mg, 60 umol, 61% yield, 97.8% purity) was obtained as a white solid.

¹H NMR: (DMSO-d6, 400 MHz): ppm 8.40 (d, J=1.6 Hz, 1H), 8.03 (s, 1H), 7.58 (d, J=9.6 Hz, 1H), 7.34 (d, J=2.4 Hz, 1H), 7.29 (dd, J=2.4, 8.8 Hz, 1H), 7.22 (dd, J=1.6, 9.6 Hz, 1H), 7.06 (d, J=8.8 Hz, 1H), 6.48 (br. s., 1H), 4.22 (d, J=4.8 Hz, 2H), 3.88 (s, 3H). ESI m/z 332.2[M+1]⁺.

### Scheme for the Synthesis of Example 113

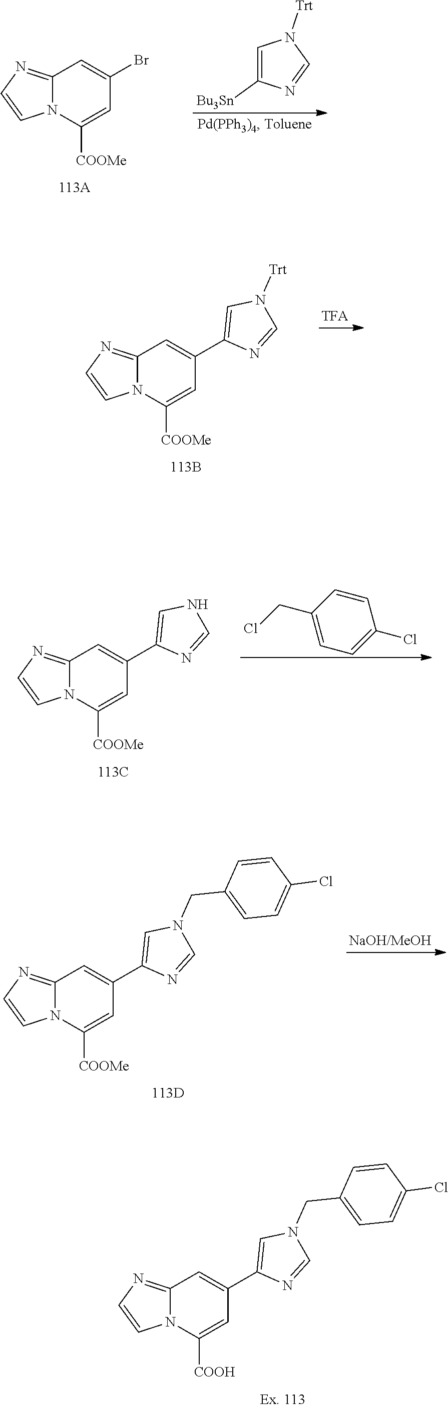

### Example 113B

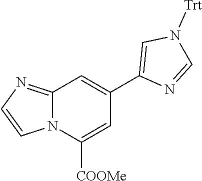

### Example 113B

A mixture of Example 113A (prepared in a manner analogous to Example 106D, 300 mg, 1.18 mmol, 1 eq), 4-(tributylstannyl)-1-trityl-1H-imidazole ([208934-35-4], 707.3 mg, 1.18 mmol, 1 eq) and Pd(PPh₃)₄ (136.4 mg, 118 umol, 0.10 eq) in dioxane (20 mL) was degassed and purged with N₂ three times. The mixture was stirred at 100° C. for 5 hours under N₂ atmosphere. The reaction was quenched with water, extracted with EtOAc (20 ml×2), and concentrated. The crude product was purified by prep-TLC. The title compound was obtained (300 mg) as a yellow oil. ESI m/z 485.2 [M+1]⁺.

### Example 113C

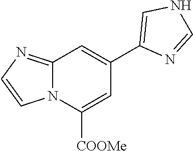 [SMILES: COC(=O)c1cc(-c2c[nH]cn2)cc2nccn12]

### Example 113C

To a solution of Example 113B (200 mg, 413 umol, 1 eq) in CHCl₃ (1 mL) was added TFA (306 uL, 4.13 mmol, 10 eq) and the mixture was stirred at 25° C. for 0.1 hour under N₂ atmosphere. The solvent was removed in vacuo to give the crude product (100 mg) as an oil which was used in the next step without further purification.

### Example 113D

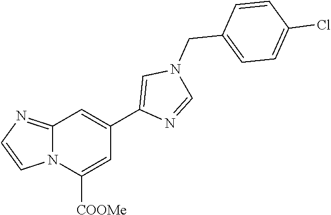 [SMILES: COC(=O)c1cc(-c2cn(Cc3ccc(Cl)cc3)cn2)cc2nccn12]

### Example 113D

A mixture of Example 113C (200 mg, 826 umol, 1 eq), 1-chloro-4-(chloromethyl)benzene (199.4 mg, 1.24 mmol, 1.5 eq) and K₂CO₃ (171.2 mg, 1.24 mmol, 1.5 eq) in DMF (1 mL) was degassed and purged with N₂ 3 times. The mixture was stirred at 80° C. for 2 hour under N₂ atmosphere. The DMF was removed in vacuo. The resulting residue was dissolved in EtOAc (50 ml), washed with water and concentrated to give crude Example 113D (200 mg) which was used in the next step without further purification. ESI m/z 367.0 [M+1]⁺.

### Example 113

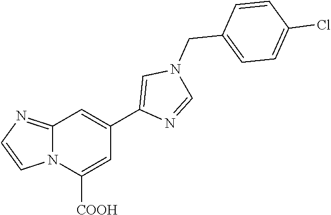 [SMILES: O=C(O)c1cc(-c2cn(Cc3ccc(Cl)cc3)cn2)cc2nccn12]

### Example 113

To a solution of Example 113D (150 mg, 409 umol, 1 eq) in MeOH (2 mL) and water (0.5 mL) was added NaOH (65.4 mg, 1.64 mmol, 4 eq). The mixture was stirred at 25° C. for 1 hour under N₂ atmosphere. The reaction was neutralized with HCl to pH 7 and concentrated. The crude product was purified by prep-HPLC (basic buffer) to provide the title compound (18.0 mg, 12.5%) as a white solid. ¹H NMR (400 MHz, CD₃OD) δ=8.84 (s, 1H), 8.01-8.01 (m, 1H), 8.01 (s, 1H), 7.92 (s, 1H), 7.87 (s, 1H), 7.70 (s, 1H), 7.56 (s, 1H), 7.46-7.40 (m, 2H), 7.38-7.33 (m, 2H), 5.30 (s, 3H) ESI m/z 353.0 [M+1]⁺.

### Scheme for the Synthesis of Example 114

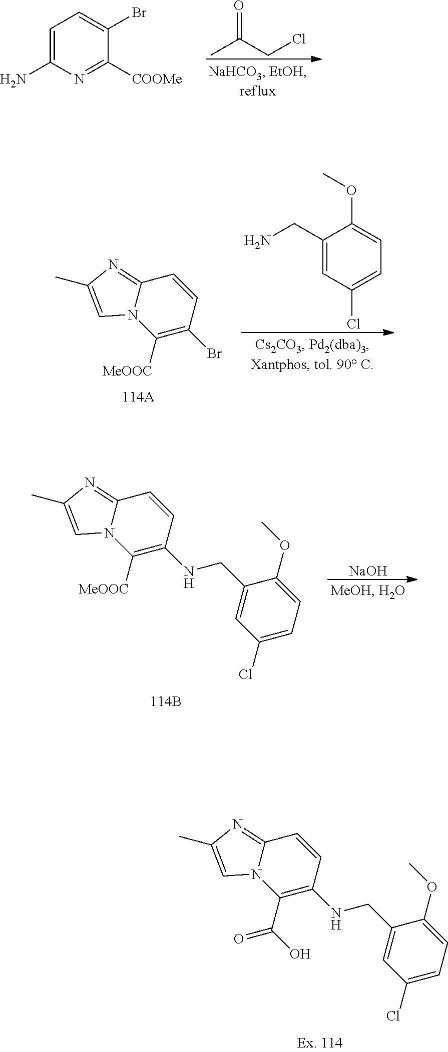

### Example 114A

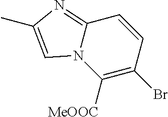

### Example 114A

To a solution of methyl 6-amino-3-bromopicolinate (200 mg, 866 umol, 1 eq) in EtOH (50 mL) was added NaHCO₃ (124 mg, 1.47 mmol, 1.7 eq) and 1-chloropropan-2-one (2.35 g, 25.4 mmol, 3.00 mL, 29.3 eq). The reaction was stirred at 90° C. for 24 hr. The reaction was cooled to 25° C. and concentrated in vacuo. To the residue was added water (50 mL). The aqueous phase was extracted with ethyl acetate (50 mL*3). The combined organic phase was washed with brine (50 mL*2), dried with anhydrous Na₂SO₄, filtered and concentrated in vacuo. The residue was purified by Prep-TLC (Petroleum ether/Ethyl acetate=1/1). Example 114A (120 mg crude) was obtained as a brown oil. ESI m/z 269[M+1]⁺.

### Example 114B

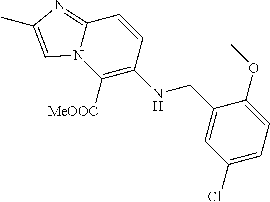 [SMILES: COC(=O)c1c(NCc2cc(Cl)ccc2OC)ccc2nc(C)cn12]

### Example 114B

To a solution of Example 114A (120 mg, 446 umol, 1 eq) and 5-chloro-2-methoxybenzyl amine (77 mg, 446 μmol, 1 eq) in toluene (1 mL) was added Cs₂CO₃ (291 mg, 892 μmol, 2 eq), Pd(dba)₂ (25.6 mg, 44.6 μmol, 0.1 eq), Xantphos (51.6 mg, 89.2 umol, 0.2 eq) under N₂. The reaction was stirred at 100° C. for 12 hr. The reaction was cooled to 25° C. and water (10 mL) was added. The aqueous phase was extracted with ethyl acetate (20 mL*3). The combined organic phase was washed with brine (20 mL*2), dried with anhydrous Na₂SO₄, filtered and concentrated in vacuo. The residue was purified by Prep-TLC (Petroleum ether/Ethyl acetate=1/1), Example 114B (50 mg, 139 umol, 31% yield) was obtained as a brown solid. ESI m/z 360.2[M+1]⁺.

### Example 114

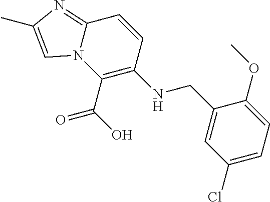 [SMILES: COc1ccc(Cl)cc1CNc1ccc2nc(C)cn2c1C(=O)O]

### Example 114

To a solution of Example 114B (50 mg, 139 umol, 1 eq) in MeOH (1 mL) and H₂O (200 uL) was added NaOH (11 mg, 278 umol, 2 eq). The reaction was stirred at 80° C. for 1 h. The reaction was cooled to 25° C. and concentrated in vacuo. The aqueous solution was adjusted to pH=5 and filtered. The solid was collected and purified by Prep-HPLC (YMC-Actus ODS-AQ 150*30 5u, water (0.1% TFA)-ACN). Example 114 (23.7 mg, 61 umol, 44% yield, 89% purity) was obtained as a yellow solid. ¹H NMR: (MeOD, 400 MHz): ppm 8.85 (s, 1H), 7.74 (d, J=10.0 Hz, 1H), 7.62 (d, J=10.0 Hz, 1H), 7.30-7.23 (m, 2H), 7.02 (d, J=9.2 Hz, 1H), 4.62 (s, 2H), 3.91 (s, 3H), 2.51 (s, 3H). ESI m/z 346.2[M+1]⁺.

### Scheme for the Synthesis of Example 115

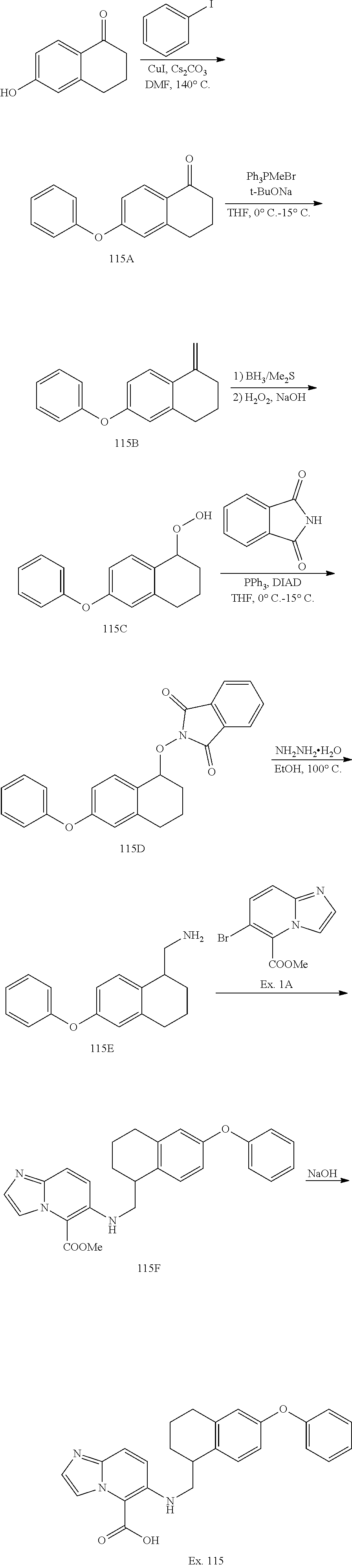

### Example 115A

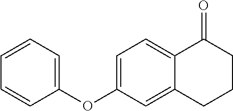 [SMILES: O=C1CCCc2cc(Oc3ccccc3)ccc21]

### Example 115A

A mixture of 6-hydroxy-3,4-dihydronaphthalen-1 (2H)-one (7.00 g, 43.16 mmol, 1 eq), iodobenzene (17.61 g, 86.32 mmol, 9.62 mL, 2 eq), CuI (822 mg, 4.32 mmol, 0.1 eq), Cs₂CO₃ (14.06 g, 43.16 mmol, 1 eq) and N,N′-dimethylethane-1,2-diamine (760 mg, 8.63 mmol, 928 uL, 0.2 eq) in DMF (20 mL) was degassed and purged with N₂ 3 times, and then the mixture was stirred at 120° C. for 20 hr under N₂ atmosphere. The reaction mixture was filtered, and the filtrate was diluted with H₂O (20 mL) and extracted with EtOAc (20 mL*2). The combined organic layers were washed with brine (30 mL*2), dried over sodium sulfate, filtered and concentrated in vacuo. The residue was purified by column chromatography (SiO₂, Petroleum ether/Ethyl acetate=50:1 to 20:1). Example 115A (2.80 g, 11.75 mmol, 27% yield) was obtained as a yellow oil.

### Example 115B

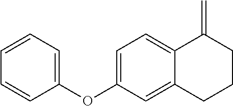 [SMILES: C=C1CCCc2cc(Oc3ccccc3)ccc21]

### Example 115B

To a solution of methyl-(triphenyl)phosphonium bromide (3.00 g, 8.39 mmol, 4 eq) in THF (15 mL) was added t-BuONa (802.6 mg, 8.35 mmol, 3 eq) at 0° C. under N₂ atmosphere. The mixture was stirred at 0° C. for 0.5 hr. To the mixture was added Example 115A (500 mg, 2.10 mmol, 1 eq) drop-wise over 15 min, then the mixture was heated to 55° C. and stirred for 2 hr. The reaction mixture was diluted with H₂O and extracted with EtOAc (40 mL*2). The combined organic layers were washed with brine (20 mL*3), dried over sodium sulfate, filtered and concentrated in vacuo. The residue was purified by column chromatography (SiO₂, Petroleum ether/Ethyl acetate=100/1 to 50:1). Example 115B (300 mg, 1.27 mmol, 60.5% yield) was obtained as a brown oil. ESI m/z 237.1 [M+1]⁺.

¹H NMR (400 MHz, CHLOROFORM-d) ppm 1.89 (m, 2H), 2.53-2.59 (m, 2H), 2.81 (t, J=6.4 Hz, 2H), 4.93 (d, J=0.8 Hz, 1H), 5.41 (s, 1H), 6.75 (d, J=2.4 Hz, 1H), 6.83 (dd, J=8.8, 2.4 Hz, 1H), 7.04 (d, J=7.6 Hz, 2H), 7.10-7.15 (m, 1H), 7.33-7.38 (m, 2H), 7.64 (d, J=8.8 Hz, 1H).

### Example 115C

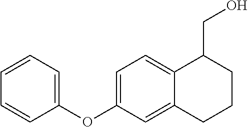 [SMILES: OCC1CCCc2cc(Oc3ccccc3)ccc21]

### Example 115C

To a solution of Example 115B (600 mg, 2.54 mmol, 1 eq) in THF (10 mL) was added BH₃-Me₂S (10 M, 2.54 mL, 10 eq) drop-wise at 0° C. The mixture was stirred at 15° C. for 3 hr and then cooled to 0° C. again. A mixture of NaOH (2.03 g, 50.78 mmol, 20 eq) dissolved in H₂O (800 uL) and H₂O₂ (5.76 g, 50.78 mmol, 4.88 mL, 30% purity, 20 eq) was added to the mixture. Stirring was continued for 3 hr at 15° C. The reaction mixture was diluted with H₂O and extracted with EtOAc (20 mL*2). The combined organic layers were washed with brine (20 mL*2), dried over sodium sulfate, filtered and concentrated in vacuo. The residue was purified by prep-TLC (SiO₂, Petroleum ether/Ethyl acetate=4:1). Example 115C (500 mg, 1.97 mmol, 77.4% yield) was obtained as a light yellow oil.

¹H NMR (400 MHz, CHLOROFORM-d) δ ppm 1.70-1.99 (m, 4H), 2.67-2.77 (m, 2 H), 2.97 (m, 1H), 3.81 (d, J=6.4 Hz, 2H), 6.76 (s, 1H), 6.81 (dd, J=8.8, 2.8 Hz, 1H), 7.01 (d, J=8.0 Hz, 2H), 7.07-7.13 (m, 1H), 7.20 (d, J=8.4 Hz, 1H), 7.30-7.37 (m, 2H).

### Example 115D

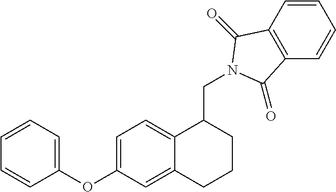 [SMILES: O=C1c2ccccc2C(=O)N1CC1CCCc2cc(Oc3ccccc3)ccc21]

### Example 115D

A mixture of Example 115C (500 mg, 1.97 mmol, 1 eq), isoindoline-1,3-dione (347.8 mg, 2.36 mmol, 1.2 eq) and PPh₃ (1.03 g, 3.94 mmol, 2 eq) in THF (10 mL) was degassed and purged with N₂ for 3 times. The mixture was cooled to 0° C. DIAD (796.7 mg, 3.94 mmol, 766 uL, 2 eq) was added dropwise under N₂ atmosphere. After addition, the mixture was warmed to 15° C. and stirred for 3 hr. The reaction mixture was concentrated in vacuo. The residue was purified by column chromatography (SiO₂, Petroleum ether/Ethyl acetate=40/1 to 20:1). Example 115D (700 mg, 1.83 mmol, 92.7% yield) was obtained as a light yellow oil.

¹H NMR (400 MHz, METHANOL-d4) ppm 1.74 (t, J=4.0 Hz, 3H), 1.97-2.09 (m, 1H), 2.60-2.88 (m, 2H), 3.26 (dd, J=9.6, 4.8 Hz, 1H), 3.72-3.92 (m, 2H), 6.72 (d, J=2.851 Hz, 2 H), 6.94 (d, J=7.6 Hz, 2H), 7.03-7.11 (m, 1H), 7.16-7.24 (m, 1H), 7.32 (t, J=8.0 Hz, 2H), 7.78-7.84 (m, 2H), 7.85-7.92 (m, 2H).

### Example 115E

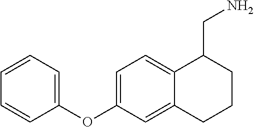 [SMILES: NCC1CCCc2cc(Oc3ccccc3)ccc21]

### Example 115E

To a solution of Example 115D (700 mg, 1.83 mmol, 1 eq) in EtOH (10 mL) was added NH₂NH₂.H₂O (1.08 g, 18.34 mmol, 1.05 mL, 85% purity, 10.02 eq). The mixture was stirred at 100° C. for 8 hr. The reaction mixture was concentrated in vacuo to remove EtOH. The residue was extracted with EtOAc, then washed with brine (20 ml*2), dried over sodium sulfate, and concentrated in vacuo. The crude product was used for the next step without further purification. Example 115E (330 mg, 1.30 mmol, 71% yield) was obtained as a light yellow oil.

¹H NMR (400 MHz, CHLOROFORM-d) δ ppm 1.69-1.78 (m, 1H), 1.80-1.94 (m, 3 H), 2.69-2.76 (m, 2H), 2.77-2.85 (m, 1H), 2.88-2.99 (m, 2H), 6.75 (s, 1H), 6.81 (dd, J=8.4, 2.4 Hz, 1H), 7.01 (d, J=7.6 Hz, 2H), 7.06-7.12 (m, 1H), 7.16 (d, J=8.4 Hz, 1H), 7.33 (t, J=8.0 Hz, 2H).

### Example 115F

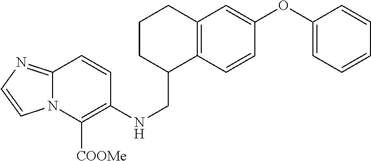 [SMILES: COC(=O)c1c(NCC2CCCc3cc(Oc4ccccc4)ccc32)ccc2nccn12]

### Example 115F

A mixture of Example 115E (180 mg, 710.5 umol, 1 eq), Example 1A (181.2 mg, 710.5 umol, 1 eq), Cs₂CO₃ (463 mg, 1.42 mmol, 2 eq), xantphos (82.2 mg, 142.1 umol, 0.2 eq) and Pd₂(dba)₃ (65 mg, 71 umol, 0.1 eq) in toluene (4 mL) was degassed and purged with N₂ 3 times. The mixture was stirred at 105° C. for 10 hr under N₂ atmosphere. The reaction mixture was filtered and concentrated in vacuo. The residue was purified by prep-TLC (SiO₂, Petroleum ether/Ethyl acetate=1:2). Example 115F (230 mg, 538 umol, 75.7% yield) was obtained as a brown oil. ESI m/z 428.1 [M+1]⁺.

### Example 115

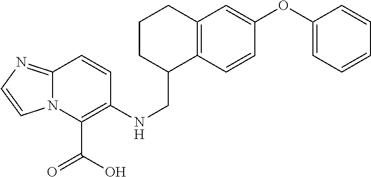 [SMILES: O=C(O)c1c(NCC2CCCc3cc(Oc4ccccc4)ccc32)ccc2nccn12]

### Example 115

To a solution of Example 114F (230 mg, 538 umol, 1 eq) in MeOH (2 mL) and H₂O (1 mL) was added NaOH (43 mg, 1.08 mmol, 2 eq). The mixture was stirred at 70° C. for 2 hr. The reaction mixture was filtered and concentrated in vacuo. The resultant mixture was adjusted to pH=4 with 2 M HCl solution, then filtered. The filter cake was washed with H₂O and dried in vacuo. Example 115 (139 mg, 336.2 umol, 62.5% yield) was obtained as a brown solid. ESI m/z 414.1 [M+1]⁺.

¹H NMR (400 MHz, METHANOL-d4) ppm 1.73-1.85 (m, 1H), 1.90-2.05 (m, 3H), 2.73-2.88 (m, 2H), 3.22 (d, J=5.2 Hz, 1H), 3.65-3.80 (m, 2H), 6.71-6.81 (m, 2H), 6.94 (d, J=8.0 Hz, 2H), 7.06-7.14 (m, 1H), 7.25-7.39 (m, 3H), 7.79-7.89 (m, 1H), 7.89-7.98 (m, 2 H), 8.98 (d, J=1.6 Hz, 1H).

### Scheme for the Synthesis of Example 116

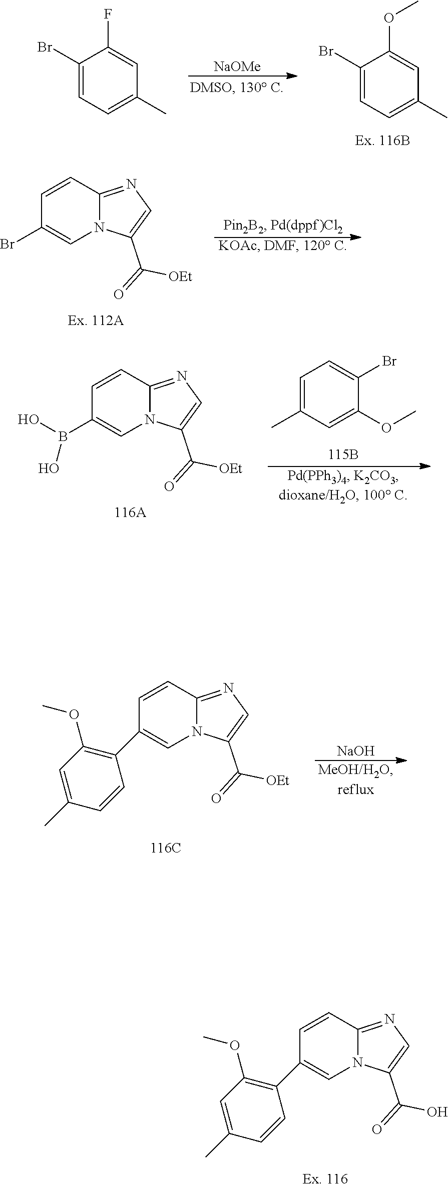

### Example 116A

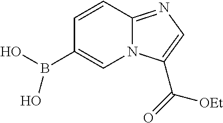 [SMILES: CCOC(=O)c1cnc2ccc(B(O)O)cn12]

### Example 116A

To a solution of Example 112A (900 mg, 3.34 mmol, 1 eq) and Pin₂B₂ (1.70 g, 6.68 mmol, 2 eq) in DMF (10 mL) was added potassium acetate (656.5 mg, 6.69 mmol, 2 eq) and bis(diphenylphosphino)ferrocene)palladium (244.7 mg, 334.5 umol, 0.1 eq). The reaction was stirred at 120° C. for 12 hrs under nitrogen. The reaction was cooled to 25° C. Water was added and the aqueous phase was extracted with ethyl acetate (100 mL*3). The combined organic phases were washed with brine (100 mL*2), dried over anhydrous sodium sulfate, filtered and concentrated in vacuo. Example 116A (1.00 g crude) was obtained as a brown oil which was used in the next step directly. ESI m/z 235.1[M+1]⁺.

### Example 116B

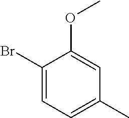

### Example 116B

To a solution of 1-bromo-2-fluoro-4-methylbenzene (1.00 g, 5.29 mmol, 1 eq) in DMSO (10 mL) was added sodium methanolate (2.86 g, 52.90 mmol, 10 eq). The mixture was stirred at 130° C. for 12 h then cooled to 25° C. Water (50 mL) was added and the aqueous phase was extracted with ethyl acetate (50 mL*3). The combined organic phases were washed with brine (50 mL*2), dried over anhydrous sodium sulfate, filtered and concentrated in vacuo. Example 116B (700 mg, yield=65.8%) was obtained as colorless oil.

¹H NMR: (CDCl3, 400 MHz): ppm 7.40 (d, J=8.0 Hz, 1H), 6.73 (s, 1H), 6.67 (d, J=8.0 Hz, 1H), 3.89 (s, 3H), 2.33 (s, 3H).

### Example 116C

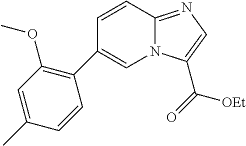 [SMILES: CCOC(=O)c1cnc2ccc(-c3ccc(C)cc3OC)cn12]

### Example 116C

To a solution of Example 116A (1.00 g, 3.16 mmol, 1 eq) and Example 116B (763 mg, 3.80 mmol, 1.20 eq) in water (1 mL) and dioxane (10 mL) was added potassium carbonate (874.3 mg, 6.33 mmol, 2 eq) and tetrakis(triphenylphosphine) palladium (365.5 mg, 316.3 umol, 0.10 eq). The reaction was stirred at 100° C. for 2 h under nitrogen. The reaction was cooled to 25° C. and concentrated in vacuo. To the residue was added water (50 mL) and the aqueous phase was extracted with ethyl acetate (100 mL*3). The combined organic phases were washed with brine (50 mL*2), dried over anhydrous sodium sulfate, filtered and concentrated in vacuo. The residue was purified by prep-TLC. Example 116C (800 mg, yield=81.6%) was obtained as a colorless oil. ESI m/z 311.1[M+1]⁺.

### Example 116

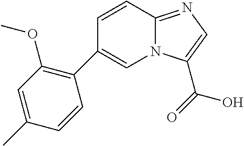 [SMILES: COc1cc(C)ccc1-c1ccc2ncc(C(=O)O)n2c1]

### Example 116

To a solution of Example 116C (300 mg, 966.7 umol, 1 eq) in methanol (10 mL) and water (2 mL) was added sodium hydroxide (77.3 mg, 1.93 mmol, 2 eq). The reaction was stirred at 80° C. for 2 h. The reaction was cooled to 25° C. and concentrated in vacuo. The aqueous solution was adjusted to pH=3 with hydrochloric acid (2 mL, 2M). The resulting suspension was filtered and the solid collected and dried in vacuo. Example 116 (100 mg, yield=36.3%) was obtained as a white solid.

¹H NMR: (DMSO-d6, 400 MHz): ppm 9.38 (s, 1H), 8.33 (s, 1H), 7.83 (d, J=9.2 Hz, 1H), 7.73 (dd, J₁=1.2, J₂=9.2 Hz, 1H), 7.32 (d, J=7.6 Hz, 1H), 7.02 (s, 1H), 6.92 (d, J=7.6 Hz, 1H), 3.81 (s, 3H), 2.38 (s, 3H). ESI m/z 283.2 [M+1]⁺.

### Scheme for the Synthesis of Example 117

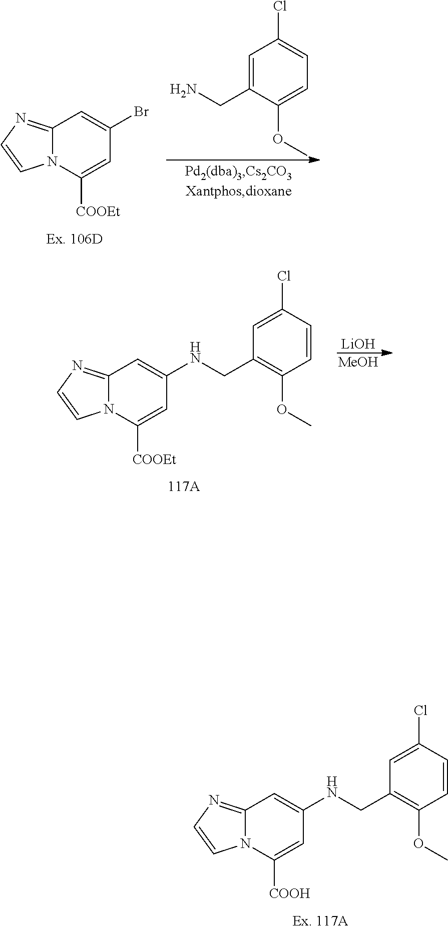

### Example 117A

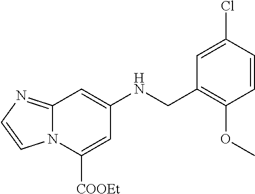 [SMILES: CCOC(=O)c1cc(NCc2cc(Cl)ccc2OC)cc2nccn12]

### Example 117A

To a solution of Example 106D (70 mg, 260 umol, 1 eq) in dioxane (5 mL) was added (5-chloro-2-methoxyphenyl)methanamine (53.6 mg, 312 umol, 1.2 eq), Xantphos (30 mg, 52 umol, 0.2 eq), Pd₂(dba)₃ (23.8 mg, 26 umol, 0.1 eq) and Cs₂CO₃ (169.5 mg, 520.3 umol, 2 eq). The reaction was stirred at 95° C. for 12 hours under N₂. The reaction was filtered and concentrated in vacuo to give Example 117A (100 mg crude) as yellow oil. It was used directly for next step.

### Example 117

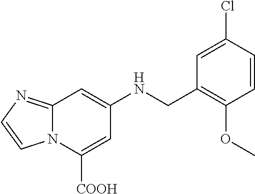 [SMILES: COc1ccc(Cl)cc1CNc1cc(C(=O)O)n2ccnc2c1]

### Example 117

To a solution of Example 117A (100 mg, 278 umol, 1 eq) in methanol (4 mL) was added LiOH.H₂O (2 M, 278 uL, 2 eq). The reaction was stirred at 20° C. for 12 hours. The reaction was concentrated in vacuo. Water (2 mL) was added and the mixture was adjusted to pH 2 with HCl (1M, 2 mL). The residue was purified by Prep-HPLC to give Example 117 (12.0 mg, yield=13%) as a yellow solid. ESI m/z 332.1 [M+1].

¹H NMR (400 MHz, CD3OD) ppm 8.67 (d, J=2.0 Hz, 1H), 7.72 (d, J=2.0 Hz, 1H), 7.59 (d, J=2.4 Hz, 1H), 7.33-7.27 (m, 2H), 7.07-7.02 (m, 1H), 6.60 (s, 1H), 4.48 (s, 2H), 3.93 (s, 3H).

### Scheme for the Synthesis of Example 118

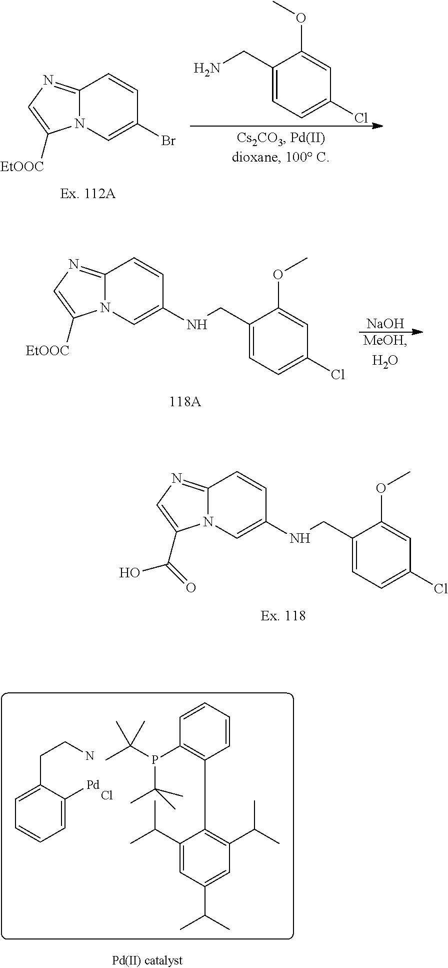

### Example 118A

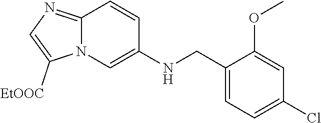 [SMILES: CCOC(=O)c1cnc2ccc(NCc3ccc(Cl)cc3OC)cn12]

### Example 118A

To a solution of Example 112A (130 mg, 483 umol, 1 eq) and (4-chloro-2-methoxyphenyl)methanamine (117 mg, 580 umol, 1.2 eq) in dioxane (2 mL) was added cesium carbonate (314.8 mg, 966 umol, 2 eq) and the Pd(II) catalyst (33.2 mg, 48.3 umol, 0.1 eq) under nitrogen. The reaction was stirred at 100° C. for 12 hr. The reaction was cooled to 25° C. Water (10 mL) was added and the aqueous phase was extracted with ethyl acetate (20 mL*3). The combined organic phases were washed with brine (20 mL*2), dried with anhydrous sodium sulfate, filtered and concentrated in vacuo. The residue was purified by prep-TLC. Example 118A (65 mg, yield=37.4%) was obtained as a brown solid. ESI m/z 360.1 [M+1]⁺.

### Example 118

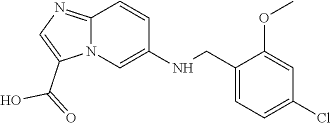 [SMILES: COc1cc(Cl)ccc1CNc1ccc2ncc(C(=O)O)n2c1]

### Example 118

To a solution of Example 118A (65 mg, 180.7 umol, 1 eq) in methanol (2 mL) and water (400 uL) was added sodium hydroxide (14.5 mg, 361.3 umol, 2 eq). The reaction mixture was stirred at 80° C. for 1 h, and then cooled to 25° C. The resulting mixture was concentrated in vacuo. The aqueous solution was adjusted to pH=8 with hydrochloric acid (1M, 2 mL). The aqueous solution was purified by prep-HPLC to provide Example 118 (14.5 mg, yield=23.8%) as a yellow solid.

¹H NMR: (CD₃OD, 400 MHz): ppm 8.88 (s, 1H), 7.91 (s, 1H), 7.39-7.31 (m, 2H), 7.08 (dd, J=2.0, 9.6 Hz, 1H), 7.00 (s, 1H), 6.91 (d, J=8.0 Hz, 1H), 4.30 (s, 2H), 3.91 (s, 3H). ESI m/z 332.0 [M+1]⁺.

### Scheme for the Synthesis of Example 119

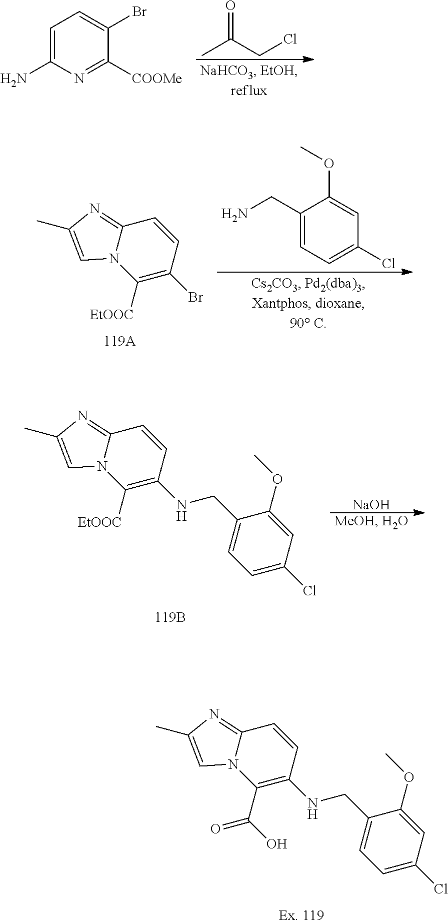

### Example 119A

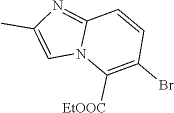 [SMILES: CCOC(=O)c1c(Br)ccc2nc(C)cn12]

### Example 119A

To a solution of methyl 6-amino-3-bromopicolinate (1.00 g, 4.33 mmol, 1 eq) in ethanol (50 mL) was added sodium bicarbonate (618 mg, 7.36 mmol, 1.7 eq) and 1-chloropropan-2-one (2.47 g, 26.7 mmol, 6.2 eq). The reaction was stirred at 90° C. for 12 hr. The reaction was cooled to 25° C. and concentrated in vacuo. To the residue was added water (50 mL) and the aqueous phase was extracted with ethyl acetate (50 mL*3). The combined organic phases were washed with brine (50 mL*2), dried over anhydrous sodium sulfate, filtered and concentrated in vacuo. The residue was purified by prep-TLC. Example 119A (400 mg, yield=34.3%) was obtained as a brown solid. ESI m/z 283.0 [M+1]⁺.

### Example 119B

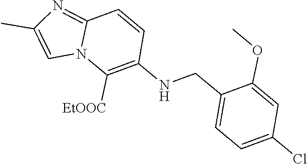 [SMILES: CCOC(=O)c1c(NCc2ccc(Cl)cc2OC)ccc2nc(C)cn12]

### Example 119B

To a solution of Example 119A (200 mg, 743 umol, 1 eq) and 4-chloro-2-methoxybenzyl amine (153 mg, 899 umol, 1.2 eq) in dioxane (5 mL) was added cesium carbonate (484 mg, 1.49 mmol, 2 eq), Xantphos (86 mg, 149 umol, 0.2 eq) and Pd₂(dba)₃ (68 mg, 74 umol, 0.1 eq) under nitrogen. The reaction was stirred at 90° C. for 12 hr. The reaction was cooled to 25° C. and water (10 mL) was added. The aqueous phase was extracted with ethyl acetate (20 mL*3). The combined organic phases were washed with brine (20 mL*2), dried over anhydrous sodium sulfate, filtered and concentrated in vacuo. The residue was purified by prep-TLC. Example 119B (105 mg, yield=39%) was obtained as a brown solid. ESI m/z 374.1 [M+1]⁺.

### Example 119

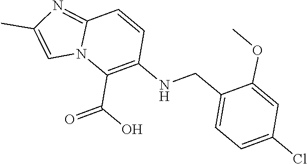 [SMILES: COc1cc(Cl)ccc1CNc1ccc2nc(C)cn2c1C(=O)O]

### Example 119

To a solution of Example 119B (105 mg, 292 umol, 1 eq) in methanol (1 mL) and water (200 uL) was added sodium hydroxide (23.4 mg, 584 umol, 2 eq). The reaction was stirred at 25° C. for 1 h. The reaction was adjusted to pH=10 with acetic acid (1 M, 2 mL) and concentrated in vacuo. The aqueous solution was purified by prep-HPLC and lyophilized. Example 119 (17.6 mg, yield=16.5%) was obtained as a yellow solid. ¹H NMR: (CD₃OD, 400 MHz): ppm 8.65 (s, 1H), 7.35-7.23 (m, 2H), 7.05-6.95 (m, 2H), 6.87 (dd, J=1.2, 8.0 Hz, 1H), 4.44 (s, 2H), 3.90 (s, 3H), 2.36 (s, 3H). ESI m/z 346.0 [M+1]⁺.

### Scheme for the Synthesis of Example 120

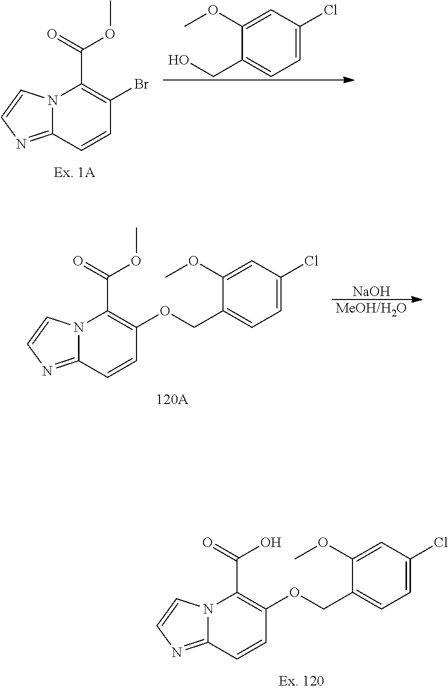 [SMILES: COC(=O)c1c(Br)ccc2nccn12 ; COc1cc(Cl)ccc1CO ; COC(=O)c1c(OCc2ccc(Cl)cc2OC)ccc2nccn12 ; "NaOH" ; "MeOH/H2O" ; COc1cc(Cl)ccc1COc1ccc2nccn2c1C(=O)O]

### Example 120A

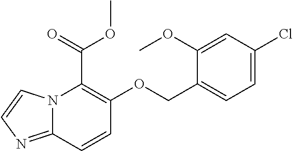 [SMILES: COC(=O)c1c(OCc2ccc(Cl)cc2OC)ccc2nccn12]

### Example 120A

A mixture of Example 1A (50 mg, 196 umol, 1 eq), (4-chloro-2-methoxyphenyl)methanol (40.6 mg, 235 umol, 1.2 eq), CuI (3.7 mg, 19.6 umol, 0.1 eq), 1,10-phenanthroline (7 mg, 39 umol, 0.2 eq) and cesium hydrogen carbonate (76 mg, 392 umol, 2 eq) in toluene (2 mL) was degassed and purged with N₂ 3 times. The mixture was stirred at 100° C. for 12 hour under N₂ atmosphere. The reaction mixture was filtered and the filtrate concentrated in vacuo to afford the crude product, which was purified by Prep-TLC to afford Example 120A (20 mg, yield=29.4%) as a light yellow solid. ¹H NMR: (CD₃OD, 400 MHz): ppm 8.38 (s, 1H), 7.83 (d, J=9.0 Hz, 1H), 7.65 (s, 1H), 7.51-7.43 (m, 2H), 7.04-6.98 (m, 2H), 5.22 (s, 2H), 3.95 (s, 3H), 3.83 (s, 3H).

### Example 120

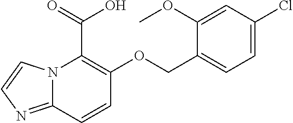 [SMILES: COc1cc(Cl)ccc1COc1ccc2nccn2c1C(=O)O]

### Example 120

To a solution of Example 120A (40 mg, 115 umol, 1 eq) in MeOH (1 mL) was added NaOH (2 M, 2.0 mL, 34.7 eq). The mixture was stirred at 25° C. for 1 hour. The reaction mixture was concentrated in vacuo to remove the MeOH. The aqueous mixture was adjusted with HCl (1M) to pH=9 and with AcOH to pH=4. The mixture was extracted with DCM (20 ml*3). The combined organic phases were dried over anhydrous sodium sulfate, filtered and concentrated in vacuo to afford the crude product as a light yellow solid. The residue was purified by Prep-TLC to afford Example 120 (15 mg, yield=39%) as a white solid.

¹H NMR: (CD₃OD, 400 MHz): 7.92 (s, 1H), 7.55 (d, J=6.8 Hz, 2H), 7.46 (d, J=9.6 Hz, 1H), 7.32 (d, J=9.6 Hz, 1H), 7.02 (s, 1H), 6.97 (d, J=8.4 Hz, 1H), 5.18 (s, 2H), 3.88 (s, 3H). ESI m/z 332.9 [M+1]⁺.

### Scheme for the Synthesis of Example 121

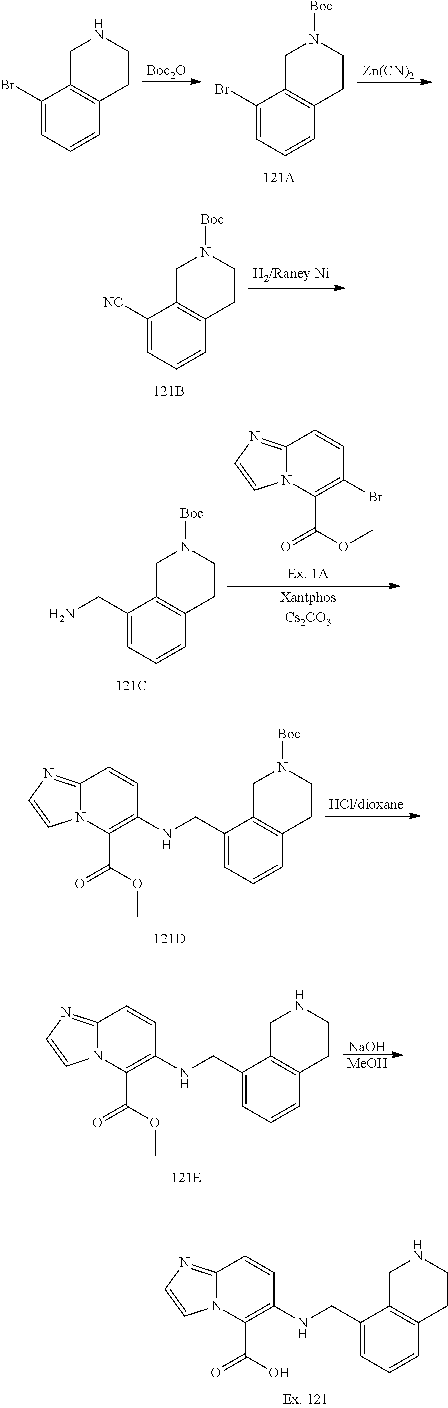

### Example 121A

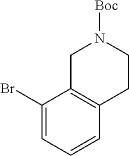 [SMILES: CC(C)(C)OC(=O)N1CCc2cccc(Br)c2C1]

### Example 121A

To a solution of 8-bromo-1,2,3,4-tetrahydroisoquinoline HCl salt (3.50 g, 14.1 mmol, 1 eq.) in tetrahydrofuran (50 mL) was added triethylamine (2.85 g, 28.2 mmol, 3.9 mL, 2 eq.) and Boc₂O (3.10 g, 14.2 mmol, 1.01 eq.). The mixture was stirred at 25° C. for 1 hour. The reaction mixture was diluted with H₂O (50 mL) and extracted with ethyl acetate (100 mL*3). The organic phases were washed with brine (100 mL), dried over anhydrous sodium sulfate and concentrated in vacuo to give Example 121A (4.20 g, yield=95.5%).

¹H NMR (400 MHz, CDCl3) δ 7.41 (d, J=7.6 Hz, 1H), 7.12-7.01 (m, 2H), 4.54 (s., 2H), 3.64 (t, J=5.2 Hz, 2H), 2.84 (t, J=5.2 Hz, 2H), 1.51 (s, 9H).

### Example 121B

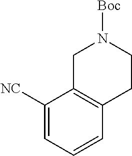 [SMILES: CC(C)(C)OC(=O)N1CCc2cccc(C#N)c2C1]

### Example 121B

A mixture of Example 121A (4.50 g, 14.4 mmol, 1 eq.), Pd₂(dba)₃ (660 mg, 720.5 umol, 0.05 eq.), DPPF (799 mg, 1.44 mmol, 0.10 eq.) and Zn(CN)₂ (1.86 g, 15.9 mmol, 1.10 eq.) in DMF (120 mL) was degassed and purged with N₂ 3 times. The mixture was stirred at 90° C. for 3 hours. The reaction mixture was filtered and diluted with H₂O (100 mL) and extracted with ethyl acetate (100 mL*2). The combined organic phases were washed with brine (100 mL), dried over anhydrous sodium sulfate and concentrated in vacuo to give a residue. The residue was purified by column chromatography to give Example 121B (3.20 g, yield=85.9%) as a white solid.

¹H NMR (400 MHz, CDCl3) δ 7.53 (d, J=7.6 Hz, 1H), 7.37 (d, J=7.6 Hz, 1H), 7.27 (d, J=7.6 Hz, 1H), 4.77 (s., 2H), 3.69 (t, J=5.6 Hz, 2H), 2.87 (t, J=5.6 Hz, 2H), 1.51 (s, 9H).

### Example 121C

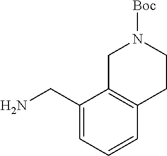 [SMILES: CC(C)(C)OC(=O)N1CCc2cccc(CN)c2C1]

### Example 121C

To a solution of Example 121B (1.00 g, 3.87 mmol, 1 eq.) in methanol (20 mL) and NH₄OH (4 mL, 37% in water) was added Raney-Ni (33 mg, 387 umol, 0.10 eq.) and the mixture was stirred under H2 (50 psi) at 25° C. for 12 hours. The reaction was concentrated to give the crude product. The crude product was purified by column chromatography to give Example 121C (0.60 g, yield=59%).

¹H NMR (400 MHz, DMSO-d6) δ 7.25 (s, 1H), 7.16 (s., 1H), 7.01 (s., 1H), 4.53 (s., 2H), 3.67 (s., 2H), 3.54 (s., 2H), 2.77 (s., 2H), 1.43 (s., 9H). ESI m/z 263.1 [M+1]⁺.

### Example 121D

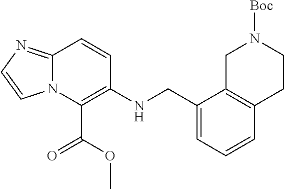 [SMILES: COC(=O)c1c(NCc2cccc3c2CN(C(=O)OC(C)(C)C)CC3)ccc2nccn12]

### Example 121D

To a solution of Example 121C (300 mg, 1.14 mmol, 1.2 eq.) and Example 1A (242.3 mg, 950 umol, 1 eq.) in dioxane (5 mL) was added Cs₂CO₃ (619 mg, 1.90 mmol, 2 eq.), Xantphos (110 mg, 190 umol, 0.2 eq.) and Pd₂(dba)₃ (87 mg, 95 umol, 0.1 eq.). The mixture was stirred at 100° C. for 12 hours under N₂. The reaction was concentrated to give a residue and the residue was purified by column chromatography to give Example 121D (400 mg, yield=90.5%). ESI m/z 437.2 [M+1]⁺.

### Example 121E

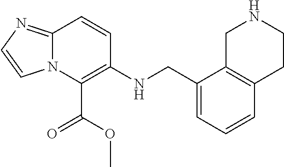 [SMILES: COC(=O)c1c(NCc2cccc3c2CNCC3)ccc2nccn12]

### Example 121E

A solution of Example 121D (400 mg, 916 umol, 1 eq.) in 4 M HCl/dioxane (20 mL) was stirred at 25° C. for 2 hours. To the reaction mixture was added saturated sodium bicarbonate solution to bring the pH to 8. The mixture was diluted with H₂O (100 mL) and extracted with DCM/MeOH=10/1 (100 mL*2). The organic phases were washed with brine (100 mL), dried over anhydrous sodium sulfate and concentrated in vacuo to give Example 121E (0.25 g, yield=81.1%). ESI m/z 337.1 [M+1]⁺.

### Example 121

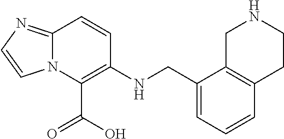 [SMILES: O=C(O)c1c(NCc2cccc3c2CNCC3)ccc2nccn12]

### Example 121

To a solution of Example 121E (250 mg, 743 umol, 1 eq.) in methanol (5 mL) was added sodium hydroxide (59.5 mg, 1.49 mmol, 2 eq.). The mixture was stirred at 80° C. for 0.5 hour. To the reaction mixture was added 2M HCl (20 mL) to bring the pH to 7 and the resulting precipitate was filtered to give Example 121 (60.0 mg, yield=25.1%). ¹H NMR (400 MHz, DMSO-d6) δ 9.14 (s, 1H), 7.61-7.55 (m, 2H), 7.27-7.19 (m, 3H), 7.13 (d, J=6.8 Hz, 1H), 4.48 (s, 2H), 4.40 (s, 2H), 3.36 (t, J=6.0 Hz, 2H), 3.03 (t, J=6.0 Hz, 2H). ESI m/z 323.0 [M+1]⁺.

### Example 122

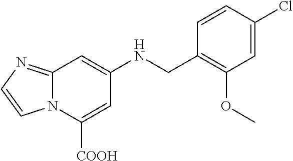 [SMILES: COc1cc(Cl)ccc1CNc1cc(C(=O)O)n2ccnc2c1]

Example 122 was prepared according to the method used for Example 117. ¹H NMR (400 MHz, CD₃C₁) 6=8.48 (s, 1H), 7.30-7.25 (m, 2H), 7.24 (d, J=2.4 Hz, 1H), 7.04 (d, J=1.4 Hz, 1H), 6.92 (dd, J=1.6, 8.0 Hz, 1H), 6.36 (d, J=2.0 Hz, 1H), 4.36 (s, 2H), 3.92 (s, 3H). ESI m/z 331.9 [M+1]⁺.

### Scheme for the Synthesis of Example 123

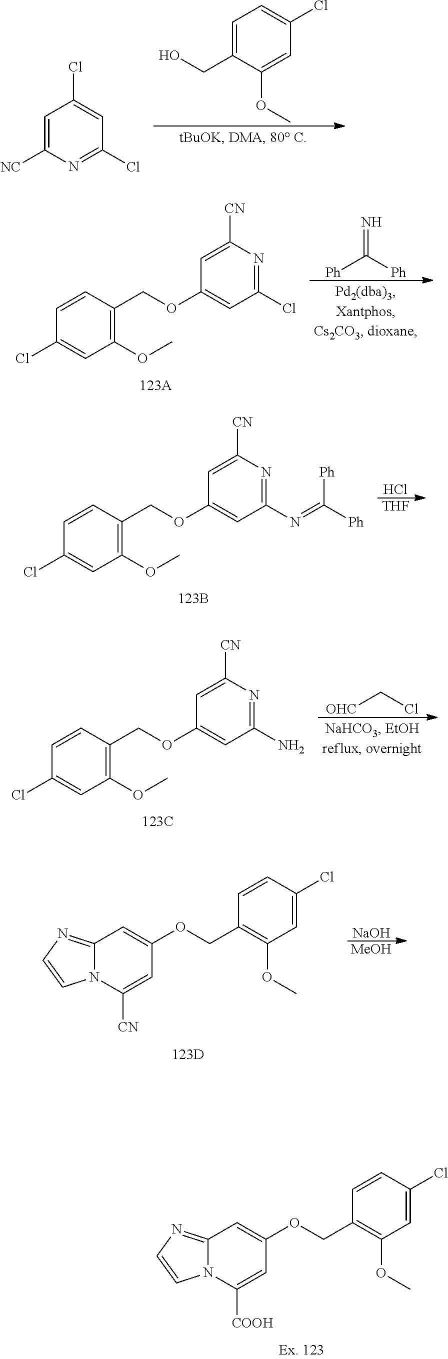

### Example 123A

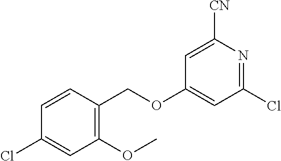 [SMILES: COc1cc(Cl)ccc1COc1cc(Cl)nc(C#N)c1]

### Example 123A

To a solution of 4,6-dichloropicolinonitrile (599 mg, 3.47 mmol, 1.2 eq) in DMA (5 mL) was added t-BuOK (389 mg, 3.47 mmol, 1.2 eq) at 25° C. The reaction was stirred at 25° C. for 0.5 hour under nitrogen, then (4-chloro-2-methoxyphenyl)methanol (500 mg, 2.89 mmol, 1 eq) was added. The reaction was heated to 80° C. 4 hours. The mixture was diluted with H₂O (50 mL) and extracted with EtOAc (50 mL). The organic layer was washed with H₂O (50 mL*2), brine (10 mL), dried over sodium sulfate, filtered and concentrated. The residue was purified by column chromatography to give Example 123A (570 mg, yield=63.8%) as a yellow solid.

¹H NMR (400 MHz, CD₃C₁) ppm=7.28-7.26 (m, 1H), 7.23 (d, J=2.4 Hz, 1H), 7.12 (d, J=2.4 Hz, 1H), 7.00 (dd, J=2.4, 8.0 Hz, 1H), 6.95 (d, J=2.4 Hz, 1H), 5.15 (s, 2H), 3.90 (s, 3H).

### Example 123B

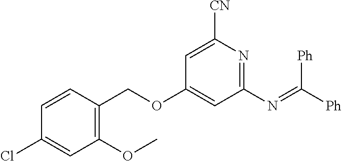 [SMILES: COc1cc(Cl)ccc1COc1cc(C#N)nc(N=C(c2ccccc2)c2ccccc2)c1]

### Example 123B

To a solution of Example 123A (570 mg, 1.84 mmol, 1 eq) in dioxane (5 mL) was added diphenylmethanimine (501 mg, 2.77 mmol, 464 uL, 1.5 eq), Xantphos (213 mg, 369 umol, 0.2 eq), Pd₂(dba)₃ (169 mg, 184 umol, 0.1 eq) and Cs₂CO₃ (1.20 g, 3.69 mmol, 2 eq). The reaction was stirred at 80° C. for 10 hours under N₂. The reaction was filtered and concentrated in vacuo. The residue was purified by column chromatography to give the Example 123B (370 mg, yield=31.5%) as yellow oil. ESI m/z 454.1 [M+1].

### Example 123C

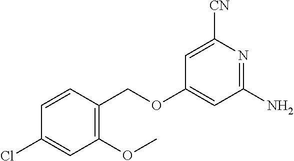 [SMILES: COc1cc(Cl)ccc1COc1cc(N)nc(C#N)c1]

### Example 123C

To a solution of Example 123B (370 mg, 815 umol, 1 eq) in THF (10 mL) was added HCl (1 M, 815 uL, 1 eq). The reaction was stirred at 20° C. for 0.5 hours. The mixture was adjusted to pH=10 with aq. Na₂CO₃. The resulting mixture was extracted with EtOAc (20 mL*2). The combined organic layers were washed with H₂O (10 mL), brine (10 mL), dried over sodium sulfate, filtered and concentrated. The residue was treated with EtOAc (0.5 mL) and PE (10 mL), and then filtered to give Example 123C (250 mg, yield=84.6%) as a yellow solid. ESI m/z 289.9 [M+1].

### Example 123D

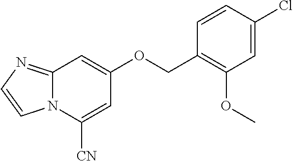 [SMILES: COc1cc(Cl)ccc1COc1cc(C#N)n2ccnc2c1]

### Example 123D

To a solution of Example 123C (250 mg, 862.9 umol, 1 eq) in EtOH (10 mL) was added 2-chloroacetaldehyde (2.54 g, 12.9 mmol, 2.08 mL, 15 eq) and NaHCO₃ (123 mg, 1.47 mmol, 1.7 eq). The reaction was stirred at 80° C. for 12 hours. The mixture was concentrated in vacuo. The residue was adjusted to pH=10 with aq. Na₂CO₃. The resulting mixture was extracted with ethyl acetate (20 mL*2). The combined organic phases were washed with brine (20 mL), dried over sodium sulfate, filtered and concentrated. The residue was purified by column chromatography to give Example 123D (200 mg, yield=67%) as a yellow solid. ESI m/z 314.0 [M+1].

### Example 123

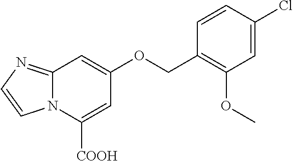 [SMILES: COc1cc(Cl)ccc1COc1cc(C(=O)O)n2ccnc2c1]

### Example 123

To a solution of Example 123D (150 mg, 478 umol, 1 eq) in MeOH (4 mL) and H₂O (1 mL) was added NaOH (38.3 mg, 956 umol, 2 eq). The reaction was stirred at 80° C. for 4 hours. The mixture was concentrated in vacuo. The residue was adjusted to pH=3 with 1 N HCl and filtered to give Example 123 (65 mg, yield=40.9%) as a brown solid. ESI m/z 333.0 [M+1].

¹H NMR (400 MHz, CD₃OD) δ ppm 8.55 (s, 1H), 7.32-7.30 (m, 2H), 7.21 (d, J=2.4 Hz, 1H), 6.96 (d, J=2.0 Hz, 1H), 6.89-6.88 (m, 2H), 5.05 (s, 2H), 3.81 (s, 3H).

### Scheme for the Synthesis of Example 124

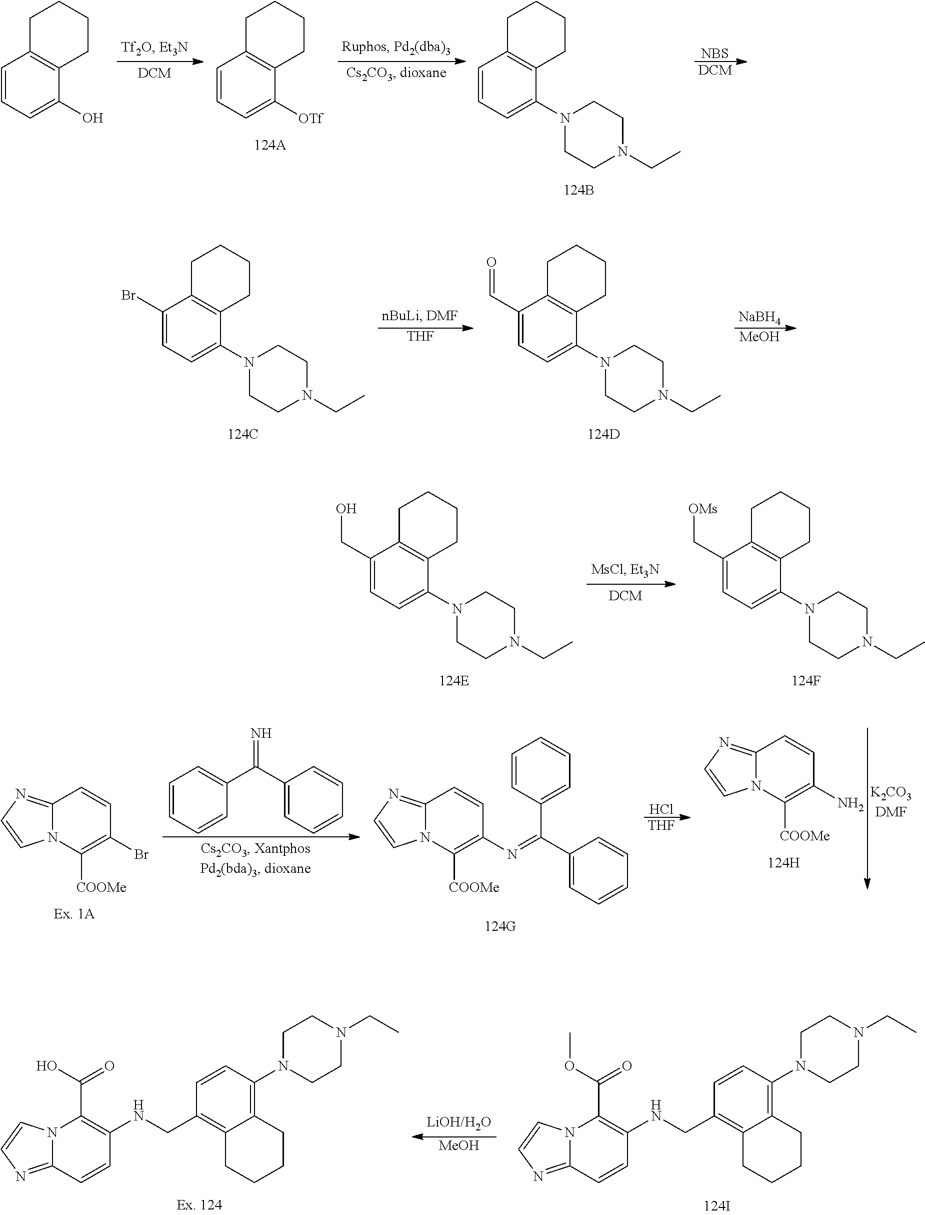

### Example 124A

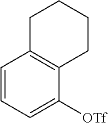 [SMILES: O=S(=O)(Oc1cccc2c1CCCC2)C(F)(F)F]

### Example 124A

To a solution of tetralin-5-ol (5 g, 33.74 mmol, 1 eq) and Et₃N (10.24 g, 101 mmol, 14 mL, 3 eq) in DCM (50 mL) at 0° C. was added dropwise trifluoromethylsulfonyl trifluoromethanesulfonate (10.47 g, 37.11 mmol, 6.12 mL, 1.1 eq). The reaction mixture was warmed to 25° C. and stirred for 4 hours. To the mixture was added water (50 mL) and the mixture extracted with DCM (25 mL*3). The combined organics were dried over anhydrous sodium sulfate and concentrated in vacuo to afford the crude product. The crude product was purified by flash chromatography on silica gel (eluent: petroleum ether) to give the product as an oil (8 g, 85% yield).

¹H NMR (400 MHz, CD₃OD) δ 7.26-7.13 (m, 2H), 7.10 (d, J=7.8 Hz, 1H), 2.84 (t, J=5.6 Hz, 2H), 2.78 (t, J=5.6 Hz, 2H), 1.89-1.76 (m, 4H).

### Example 124B

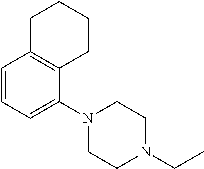 [SMILES: CCN1CCN(c2cccc3c2CCCC3)CC1]

### Example 124B

To a solution of Example 124A (4.90 g, 17.48 mmol, 1 eq), 1-ethylpiperazine (2.79 g, 24.47 mmol, 3.10 mL, 1.40 eq), Cs₂CO₃ (11.39 g, 34.96 mmol, 2 eq) and RuPhos (1.14 g, 2.45 mmol, 0.14 eq) in dioxane (50 mL) was added Pd₂(dba)₃ (800 mg, 874 μmol, 0.05 eq). The mixture was heated to 100° C. under a N₂ atmosphere for 6 hours. The mixture was cooled to RT and filtered. The filtrate was concentrated in vacuo to afford crude product. The crude product was purified by flash chromatography on silica gel (DCM/MeOH=20/1) to obtain the product as a yellow solid (1.85 g, 43% yield). ESI m/z 245.0 [M+1]⁺.

¹H NMR (400 MHz, CD₃OD) δ 7.10-7.04 (m, 1H), 6.92 (d, J=8.0 Hz, 1H), 6.84 (d, J=7.8 Hz, 1H), 3.03 (br. s, 8H), 2.87 (q, J=7.2 Hz, 2H), 2.81-2.72 (m, 4H), 1.84-1.73 (m, 4H), 1.28 (t, J=7.4 Hz, 3H).

### Example 124C

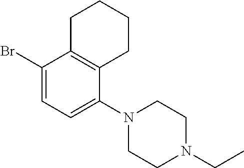 [SMILES: CCN1CCN(c2ccc(Br)c3c2CCCC3)CC1]

### Example 124C

To a solution of Example 124B (2.70 g, 11.05 mmol, 1 eq) in DCM (100 mL) was added NBS (2.16 g, 12.16 mmol, 1.10 eq) portion-wise. The mixture was stirred at 25° C. for 2 hours. The solvent was removed in vacuo to afford the crude product. The crude product was purified by flash chromatography on silica gel (DCM/MeOH=20/1) to obtain the product as a yellow solid (2.6 g, 73% yield). ESI m/z 324.9 [M+1]⁺.

¹H NMR (400 MHz, CD₃OD) δ 7.37 (d, J=8.4 Hz, 1H), 6.87 (d, J=8.4 Hz, 1H), 3.00-2.96 (m, 4H), 2.78-2.67 (m, 8H), 1.88-1.69 (m, 6H), 1.24-1.20 (m, 3H).

### Example 124D

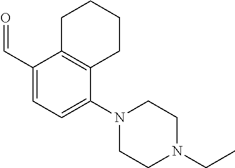 [SMILES: CCN1CCN(c2ccc(C=O)c3c2CCCC3)CC1]

### Example 124D

To a solution of Example 124C (600 mg, 1.86 mmol, 1 eq) in THF (20 mL) was added n-BuLi (2.5 M, 1.86 mL, 2.50 eq) at −78° C. After 30 min, DMF (1.36 g, 18.60 mmol, 1.43 mL, 10 eq) was added to the solution and the reaction stirred at −78° C. for 1 hour, then warmed to 0° C. for 30 min. Ice water was slowly added to the solution at 0° C., and the mixture extracted with EA (50 mL*3). The organics were dried with anhydrous sodium sulfate and concentrated in vacuo to afford the crude product. The crude product was purified by flash chromatography on silica gel (DCM/MeOH=100/1, 0.25% Et₃N) to obtain the product as a yellow oil (0.36 g, 71% yield). ESI m/z 273.0 [M+1]⁺.

¹H NMR (400 MHz, CD₃OD) δ 10.10 (s, 1H), 7.67 (d, J=8.2 Hz, 1H), 7.05 (d, J=8.2 Hz, 1H), 3.23 (t, J=6.6 Hz, 2H), 3.04 (t, J=4.2 Hz, 4H), 2.80-2.75 (m, 2H), 2.68 (br. s., 4H), 2.57-2.50 (m, 2H), 1.90-1.81 (m, 2H), 1.76-1.69 (m, 2H), 1.20-1.14 (m, 3H).

### Example 124E

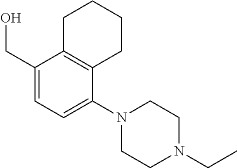 [SMILES: CCN1CCN(c2ccc(CO)c3c2CCCC3)CC1]

### Example 124E

To a solution of Example 124D (350 mg, 1.28 mmol, 1 eq) in MeOH (5 mL) was added NaBH₄ (97 mg, 2.56 mmol, 2 eq) slowly. The reaction was stirred for 2 hours at 25° C. The solvent was removed in vacuo. Water (10 mL) was added. The mixture was extracted with ethyl acetate. The organics were dried with anhydrous sodium sulfate and concentrated in vacuo to afford the crude product. The crude product was purified by flash chromatography on silica gel (DCM/MeOH) to obtain the product as an oil. (150 mg, 42.7% yield). ESI m/z 275.0 [M+1]⁺.

### Example 124F

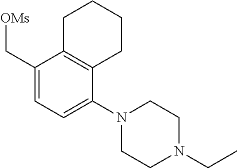 [SMILES: CCN1CCN(c2ccc(COS(C)(=O)=O)c3c2CCCC3)CC1]

### Example 124F

To a solution of Example 124E (180 mg, 656 μmol, 1 eq) and Et₃N (132.76 mg, 1.31 mmol, 182 μl, 2 eq) in DCM (2 mL) was added MsCl (112.71 mg, 984 μmol, 76.16 μl, 1.50 eq) at 0° C. The mixture was stirred at 0° C. for 1 hour. The solvent was removed in vacuo and the residue was used directly for the next step without further purification (200 mg crude target compound).

### Example 124G

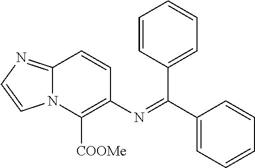 [SMILES: COC(=O)c1c(N=C(c2ccccc2)c2ccccc2)ccc2nccn12]

### Example 124G

To a solution of Example 1A (400 mg, 1.57 mmol, 1 eq) and diphenylmethanimine (313 mg, 1.73 mmol, 290 uL, 1.1 eq) in dioxane (5 mL) were added Xantphos (181.5 mg, 314 umol, 0.2 eq), Pd₂(dba)₃ (143.6 mg, 157 umol, 0.1 eq) and Cs₂CO₃ (2.04 g, 6.27 mmol, 4 eq). The mixture was degassed and purged with N₂ 3 times, then stirred at 85° C. for 8 hr under N₂ atmosphere. The reaction mixture was filtered and concentrated to give the crude product which was purified by prep-TLC. Example 124G (520 mg, yield=93.2%) was obtained as a light yellow oil. ESI m/z 356.1 [M+1]⁺.

### Example 12411

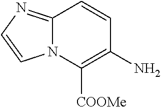 [SMILES: COC(=O)c1c(N)ccc2nccn12]

### Example 124H

To a solution of Example 124G (520 mg, 1.46 mmol, 1 eq) in THF (5 mL) was added HCl (2 M, 1.46 mL, 2 eq). The mixture was stirred at 25° C. for 1 hr. The reaction mixture was adjusted to pH=9-10 with 1M NaOH solution (10 mL) and extracted with DCM (20 mL*3). The combined organic layers were washed with H₂O (10 mL*2), dried over sodium sulfate, filtered and concentrated. The residue was triturated with PE (10 mL) and the precipitated solid was filtered and dried in vacuo. Example 124H (300 mg crude) was obtained as a yellow solid. ESI m/z 192.2 [M+1]⁺.

### Example 1241

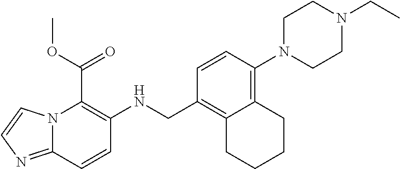 [SMILES: CCN1CCN(c2ccc(CNc3ccc4nccn4c3C(=O)OC)c3c2CCCC3)CC1]

### Example 1241

To a solution of Example 124H (51.5 mg, 269.5 umol, 0.95 eq), K₂CO₃ (117.6 mg, 851.1 umol, 3 eq) in DMF (4 mL) was added Example 128F (100 mg, 283.7 umol, 1 eq). The mixture was heated to 80° C. for 2 hours. The reaction was filtered to remove the solid and the filtrate was evaporated to afford the crude product. The crude product was purified by Prep-TLC. Example 1241 was obtained as a yellow solid (30 mg, yield=23.6%).

¹H NMR (400 MHz, CD₃OD) δ=8.44 (s, 1H), 7.50 (d, J=10.0 Hz, 1H), 7.40-7.36 (m, 1H), 7.04-6.96 (m, 2H), 6.81 (d, J=8.2 Hz, 1H), 4.40 (s, 2H), 3.92 (s, 3H), 2.85 (br. s., 4H), 2.69 (t, J=6.0 Hz, 6H), 2.57-2.51 (m, 2H), 1.80-1.73 (m, 2H), 1.70-1.62 (m, 2H), 1.09 (t, J=7.2 Hz, 3H). ESI m/z 448.0 [M+1]⁺.

### Example 124

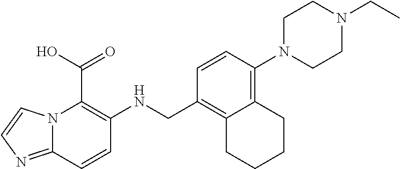 [SMILES: CCN1CCN(c2ccc(CNc3ccc4nccn4c3C(=O)O)c3c2CCCC3)CC1]

### Example 124

To a solution of Example 1241 (25 mg, 55.9 umol, 1 eq) in MeOH (5 mL) was added LiOH solution (2 M, 1 mL). The mixture was stirred at 25° C. for 2 hours. The reaction was adjusted to a pH of 7 with 2 M HCl solution and concentrated in vacuo. The resulting solid was filtered, washed with water (2*1 mL) and dried in vacuo to obtain Example 124 as a yellow solid (16 mg, yield=66.1%).

¹H NMR (400 MHz, CD₃OD) δ=9.10-8.96 (m, 1H), 7.50-7.37 (m, 2H), 7.20-7.08 (m, 2H), 6.86 (d, J=8.0 Hz, 1H), 4.37 (s, 2H), 2.94 (br. s., 4H), 2.90-2.71 (m, 8H), 2.67 (q, J=7.2 Hz, 2H), 1.84 (d, J=5.6 Hz, 2H), 1.75 (d, J=5.2 Hz, 2H), 1.21 (t, J=7.2 Hz, 3H). ESI m/z 434.0 [M+1]⁺.

### Scheme for the Synthesis of Example 125

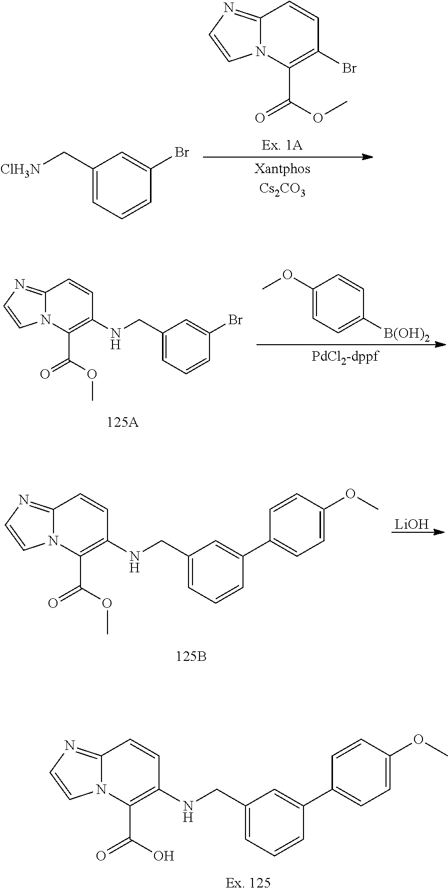

### Example 125A

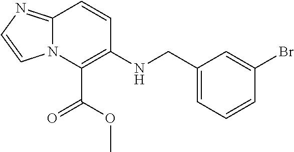 [SMILES: COC(=O)c1c(NCc2cccc(Br)c2)ccc2nccn12]

Example 125A was prepared according to the procedure used for Example 1B. ESI m/z 360.1 [M+1]⁺.

### Example 125B

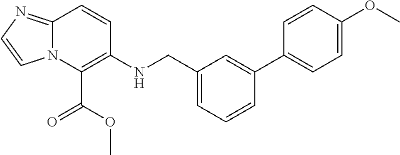 [SMILES: COC(=O)c1c(NCc2cccc(-c3ccc(OC)cc3)c2)ccc2nccn12]

### Example 125B

To a mixture of Example 125A (71 mg, 0.20 mmol) in toluene (1.5 mL) and saturated NaHCO₃ (1.5 mL) was added 4-methoxyphenyl boronic acid (45 mg, 0.30 mmol) and PdCl₂-dppf (22 mg, 0.03 mmol). The mixture was degassed 3 times and then stirred under a nitrogen atmosphere at 90° C. overnight. The mixture was cooled and diluted with water. The mixture was extracted with ethyl acetate. The organic layer was washed with brine, dried and concentrated. The residue was purified by prep TLC. ESI m/z 388.2 [M+1]⁺.

### Example 125

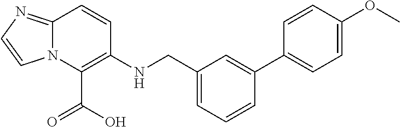 [SMILES: COc1ccc(-c2cccc(CNc3ccc4nccn4c3C(=O)O)c2)cc1]

Example 125 was prepared according to the procedure used for Example 1. ESI m/z 374.2 [M+1]⁺.

¹H NMR (500 MHz, DMSO-d6) δ ppm 7.67 (d, J=9.5 Hz, 2H), 7.61 (br s, 1H), 7.57 (d, J=8.5 Hz, 2H), 7.49 (d, J=7.5, 1H), 7.39 (t, J=7.5 Hz, 1H), 7.32 (br s, 1H), 7.27 (d, J=7.5 Hz, 1H), 7.01 (d, J=8.5 Hz, 2H), 4.64 (s, 2H), 3.79 (s, 3H).

### Example 126

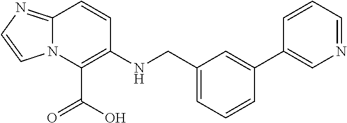 [SMILES: O=C(O)c1c(NCc2cccc(-c3cccnc3)c2)ccc2nccn12]

Example 126 was prepared from Example 125A according to the procedures for Example 125B and Example 1. ESI m/z 345.2 [M+1]⁺.

¹H NMR (500 MHz, DMSO-d6) δ ppm 9.04 (br s, 1H), 8.87 (d, J=2 Hz, 1H), 8.57 (d, J=3.5 Hz, 1H), 8.05 (d, J=7.5 Hz, 1H), 7.73 (s, 1H), 7.67 (m, 2H), 7.61 (d, J=8 Hz, 1H), 7.48 (m, 2H), 7.40 (d, J=7.5 Hz, 1H), 7.33 (d, J=10 Hz, 1H), 4.67 (s, 2H).

### Example 127

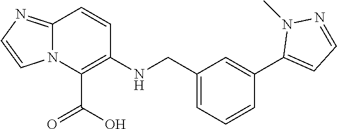 [SMILES: Cn1nccc1-c1cccc(CNc2ccc3nccn3c2C(=O)O)c1]

Example 127 was prepared from Example 124A according to the procedures for Example 125B and Example 1. ESI m/z 348.2 [M+1]⁺.

¹H NMR (500 MHz, DMSO-d6) δ ppm 9.00 (s, 1H), 7.83 (s, 1H), 7.78 (d, J=9.5 Hz, 1H), 7.45 (m, 6H), 6.37 (s, 1H), 4.70 (s, 2H), 3.81 (s, 3H).

### Scheme for the Synthesis of Example 128

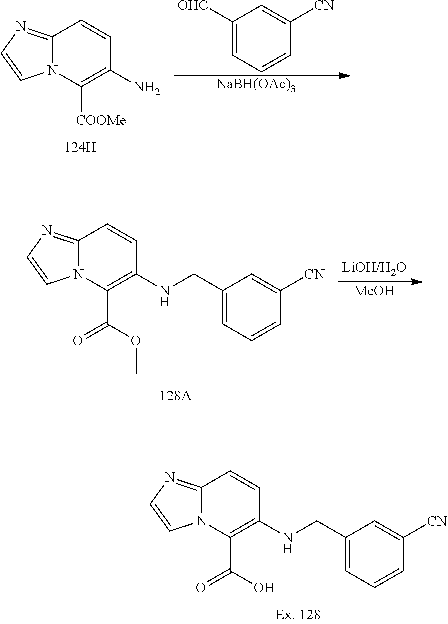 [SMILES: "MeOH" ; COC(=O)c1c(N)ccc2nccn12 ; N#Cc1cccc(C=O)c1 ; COC(=O)c1c(NCc2cccc(C#N)c2)ccc2nccn12 ; N#Cc1cccc(CNc2ccc3nccn3c2C(=O)O)c1 ; "NaBH(OAc)3"]

### Example 128A

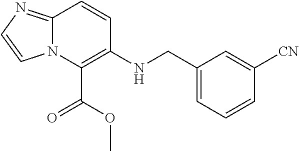 [SMILES: COC(=O)c1c(NCc2cccc(C#N)c2)ccc2nccn12]

### Example 128A

Example 124H (220 mg, 1.16 mmol), 3-cyanobenzaldehyde (228 mg, 1.74 mmol) and triacetoxyborohydride (490 mg, 2.33 mmol) were stirred together in DCM (5 mL). Acetic acid (0.2 mL) was added. The mixture was stirred overnight. The mixture was diluted with water and extracted with ethyl acetate. The organic layer was washed with brine, dried and concentrated. The residue was purified by silica gel chromatography. ESI m/z 307.2 [M+1]⁺.

### Example 128

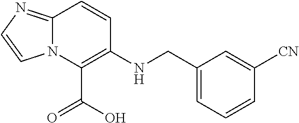 [SMILES: N#Cc1cccc(CNc2ccc3nccn3c2C(=O)O)c1]

Example 128 was prepared from Example 128A according to the procedure for Example 1. ESI m/z 293.2 [M+1]⁺.

¹H NMR (500 MHz, DMSO-d6) δ ppm 9.05 (br s, 1H), 7.68 (br s, 1H), 7.66 (d, J=9.5 Hz, 1H), 7.54 (s, 1H), 7.44 (d, J=8 Hz, 1H), 7.31 (m, 2H), 7.26 (br s, 1H), 4.60 (s, 2H).

### Scheme for the Synthesis of Example 129

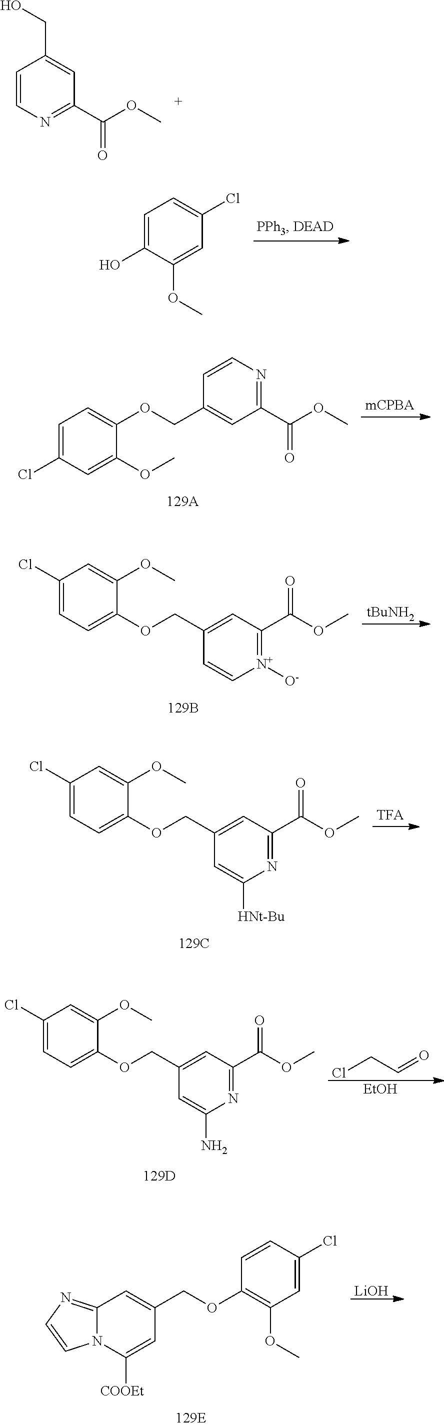

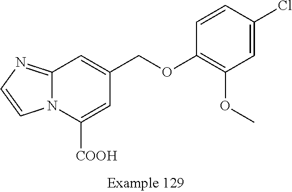 [SMILES: COc1cc(Cl)ccc1OCc1cc(C(=O)O)n2ccnc2c1]

### Example 129A

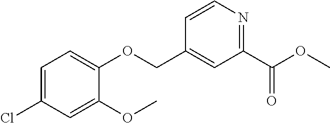 [SMILES: COC(=O)c1cc(COc2ccc(Cl)cc2OC)ccn1]

To a solution of methyl 4-(hydroxymethyl)picolinate (1.12 g, 6.68 mmol, 1 eq), 4-chloro-2-methoxyphenol (1.27 g, 8.01 mmol, 977 uL, 1.2 eq) and PPh₃ (3.50 g, 13.35 mmol, 2 eq) in THF (2 mL) was added DEAD (1.74 g, 10 mmol, 1.82 mL, 1.5 eq) at 0° C. The mixture was stirred at 0° C. for 0.5 hour, then at 25° C. for 2 hours. The solvent was removed in vacuo. The mixture was diluted with EtOAc (15 mL), washed with water (15 mL*2) and brine (15 mL). The organic phase was dried, filtered and concentrated. The residue was purified by silica gel column to give Example 129A (5.20 g) as a yellow solid, which was used directly in the next step.

### Example 129B

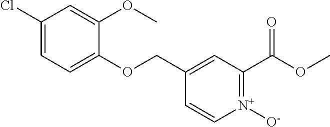 [SMILES: COC(=O)c1cc(COc2ccc(Cl)cc2OC)cc[n+]1[O-]]

To a solution of Example 129A (5.00 g, 16.25 mmol, 1 eq) in DCM (50 mL) was added m-CPBA (5.61 g, 32.5 mmol, 2 eq). The mixture was stirred at 25° C. for 16 hours. The reaction mixture was quenched with Na₂SO₃ (50 mL) and stirred for 0.5 hr. The organic phase was washed with aqueous Na₂SO₃ (50 mL), aqueous NaHCO₃ (50 mL) and brine (50 mL). The organic phase was dried, filtered and concentrated. The residue was purified by silica gel column to give Example 129B (1.91 g, yield=36.3%) as a yellow solid. ESI m/z 320.4 [M+1]⁺.

¹H NMR (400 MHz, CDCl₃) δ=8.25 (d, J=6.8 Hz, 1H), 7.69 (br d, J=2.0 Hz, 1H), 7.42 (br dd, J=2.3, 6.4 Hz, 1H), 6.92 (d, J=2.4 Hz, 1H), 6.89-6.85 (m, 1H), 6.81-6.76 (m, 1H), 5.06 (s, 2H), 4.02 (s, 3H), 3.89 (s, 3H).

### Example 129C

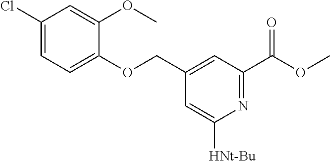

A mixture of Example 129B (1.91 g, 5.9 mmol, 1 eq), TEA (2.09 g, 20.65 mmol, 2.86 mL, 3.5 eq), PYBROP (4.13 g, 8.85 mmol, 1.5 eq) and 2-methylpropan-2-amine (647.3 mg, 8.85 mmol, 924.7 uL, 1.5 eq) was sealed in a pressure tube. The mixture was stirred at 100° C. for 16 hours. The reaction mixture was diluted with water (15 mL) and extracted with EtOAc (15 mL*3). The combined organic phases were washed with brine (15 mL), dried, filtered and concentrated. The residue was purified by prep-TLC to give Example 129C (840 mg, yield=37.6%) as a yellow solid. ESI m/z 379.1 [M+1]⁺.

¹H NMR (400 MHz, CDCl₃) δ=7.37 (s, 1H), 6.90 (d, J=2.4 Hz, 1H), 6.85-6.82 (m, 1H), 6.79-6.73 (m, 2H), 5.08 (s, 2H), 3.95 (s, 3H), 3.90 (s, 3H), 1.41 (s, 9H).

### Example 129D

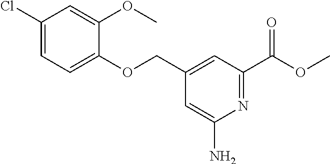 [SMILES: COC(=O)c1cc(COc2ccc(Cl)cc2OC)cc(N)n1]

Example 129C (400 mg, 1.06 mmol, 1 eq) in TFA (7.70 g, 67.5 mmol, 5.00 mL, 63.7 eq) was stirred at 80° C. for 16 hr. The mixture was diluted with DCM (2 mL) and washed with sat NaHCO₃ (2 mL*2). The organic phase was dried, filtered and concentrated. The residue was purified by prep-TLC to give Example 129D (219 mg, yield=78.1%). The crude product was used directly in the next step.

### Example 129E

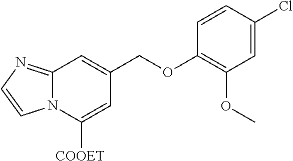

To a solution of Example 129D (219 mg, 679 umol, 1 eq) and NaHCO₃ (114 mg, 1.36 mmol, 2 eq) in EtOH (2 mL) was added 2-chloroacetaldehyde (532.7 mg, 2.7 mmol, 437 uL, 4 eq). The mixture was stirred at 80° C. for 16 hours. The solvent was removed in vacuo. The mixture was diluted with water and extracted with EtOAc (10 mL*3). The combined organic phases were washed with brine (10 mL), dried, filtered and concentrated. The residue was purified by prep-TLC to give Example 129E (244 mg, 676.3 umol, yield=99.7%) as a yellow oil. ESI m/z 361.2 [M+1]⁺.

### Example 129

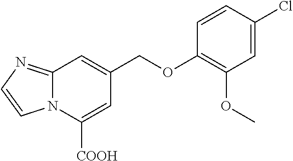 [SMILES: COc1cc(Cl)ccc1OCc1cc(C(=O)O)n2ccnc2c1]

To a solution of Example 129E (130 mg, 360.3 umol, 1 eq) in THF (2 mL) and H₂O (600 uL) was added LiOH (34.5 mg, 1.44 mmol, 4 eq). The mixture was stirred at 25° C. for 16 hours. The THF was removed in vacuo and neutralized with 1M HCl to pH=8. The resulting solid was filtered and washed with water (5 mL) to give Example 129 (61 mg, yield=47.6%) as a white solid. ESI m/z 333.1 [M+1]⁺.

¹H NMR (400 MHz, DMSO-d₆) δ=9.05 (s, 1H), 7.93 (br s, 1H), 7.76 (br d, J=13.2 Hz, 2H), 7.16-7.08 (m, 2H), 6.99-6.89 (m, 1H), 5.31-5.23 (m, 2H), 3.86 (s, 3H).

### Scheme for the Synthesis of Example 130

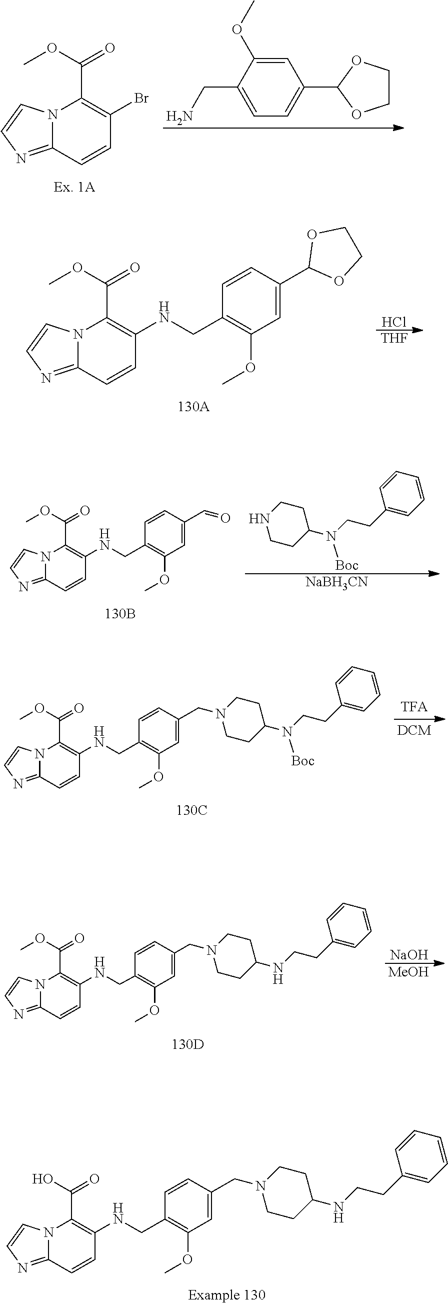

### Example 130A

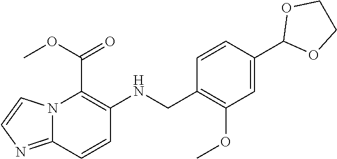 [SMILES: COC(=O)c1c(NCc2ccc(C3OCCO3)cc2OC)ccc2nccn12]

### Example 130A

To a solution of Example 1A (600 mg, 2.35 mmol, 1 eq) in dioxane (10 mL) was added (4-(1,3-dioxolan-2-yl)-2-methoxyphenyl)methanamine (984.4 mg, 4.7 mmol, 2 eq), Cs₂CO₃ (1.53 g, 4.70 mmol, 2. eq), Xantphos (272.2 mg, 470 umol, 0.20 eq) and Pd₂(dba)₃ (215.4 mg, 235 umol, 0.10 eq). The reaction was stirred at 90° C. for 12 hours. The reaction was filtered and concentrated in vacuo. The residue was purified by prep-TLC to give the title compound (457 mg, 49.8%) as a yellow solid. ESI m/z 384.0 [M+1].

### Example 130B

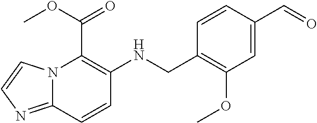 [SMILES: COC(=O)c1c(NCc2ccc(C=O)cc2OC)ccc2nccn12]

### Example 130B

To a solution of Example 130A (547 mg, 1.43 mmol, 1 eq) in THF (5 mL) was added HCl (4 M, 1.9 mL, 37.3 eq). The reaction was stirred at 20° C. for 1 hour. The reaction was concentrated in vacuo. The residue was purified by column chromatography to give the title compound (350 mg, 69%) as yellow oil. ESI m/z 340.0 [M+1].

### Example 130C

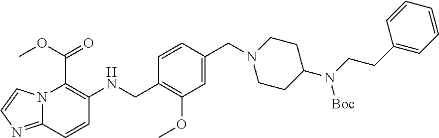 [SMILES: COC(=O)c1c(NCc2ccc(CN3CCC(N(CCc4ccccc4)C(=O)OC(C)(C)C)CC3)cc2OC)ccc2nccn12]

### Example 130C

To a solution of Example 130B (200 mg, 589.4 umol, 1 eq) in DCE (5 mL) was added tert-butyl phenethyl(piperidin-4-yl)carbamate (215.3 mg, 707.2 umol, 1.2 eq) and AcOH (33.7 uL, 589 umol, 1 eq). The reaction was stirred at 50° C. for 3 hours. NaBH(OAc)₃ (374.7 mg, 1.77 mmol, 3 eq) was added and the reaction was stirred at 40° C. for 12 hours. After cooling, the reaction mixture was diluted with DCM (20 mL) and washed with saturated brine (10 mL). The organic phase was dried over sodium sulfate and concentrated to give the title compound (200 mg, 54%) as green oil which was used directly in the next step.

### Example 130D

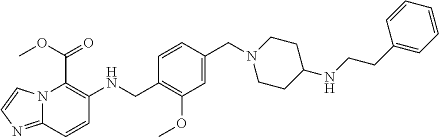 [SMILES: COC(=O)c1c(NCc2ccc(CN3CCC(NCCc4ccccc4)CC3)cc2OC)ccc2nccn12]

### Example 130D

To a solution of Example 130C (180 mg, 286.7 umol, 1 eq) in DCM (2 mL) was added TFA (500 uL, 6.75 mmol, 23.6 eq). The reaction was stirred at 20° C. for 0.5 hour. The reaction was concentrated in vacuo. The residue was diluted with ethyl acetate (10 mL) and washed with saturated sodium carbonate (10 mL×2). The combined phase was dried with anhydrous Na₂SO₄, filtered and concentrated in vacuo to give the title compound (120 mg, 78.6%) as a yellow oil. ESI m/z 528.1 [M+1].

### Example 130

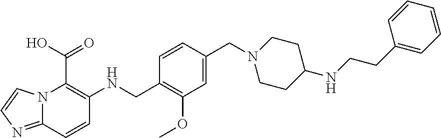 [SMILES: COc1cc(CN2CCC(NCCc3ccccc3)CC2)ccc1CNc1ccc2nccn2c1C(=O)O]

### Example 130

To a solution of Example 130D (120 mg, 227.4 umol, 1 eq) in MeOH (2 mL) was added NaOH (2 M, 1.2 mL, 10.5 eq). The reaction was stirred at 20° C. for 1 hour. The mixture was adjusted to pH 8 with HCl (2 M) and concentrated in vacuo. The residue was purified by prep-HPLC to give the title compound (37.5 mg, 31.8%) as a white solid. ESI m/z 514.3 [M+1].

¹H NMR (400 MHz, CD₃OD) δ=8.92 (s, 1H), 7.37 (d, J=2.0 Hz, 2H), 7.32-7.27 (m, 2H), 7.26-7.20 (m, 4H), 7.11 (d, J=4.0 Hz, 1H), 6.95 (s, 1H), 6.79 (d, J=7.2 Hz, 1H), 4.45 (s, 2H), 3.87 (s, 3H), 3.49 (s, 2H), 3.02-2.96 (m, 2H), 2.91 (d, J=11.6 Hz, 2H), 2.87-2.82 (m, 2H), 2.73 (s, 1H), 2.06 (t, J=12.0 Hz, 2H), 1.93 (d, J=12.0 Hz, 2H), 1.54-1.42 (m, 2H).

### Scheme for the Synthesis of Example 131

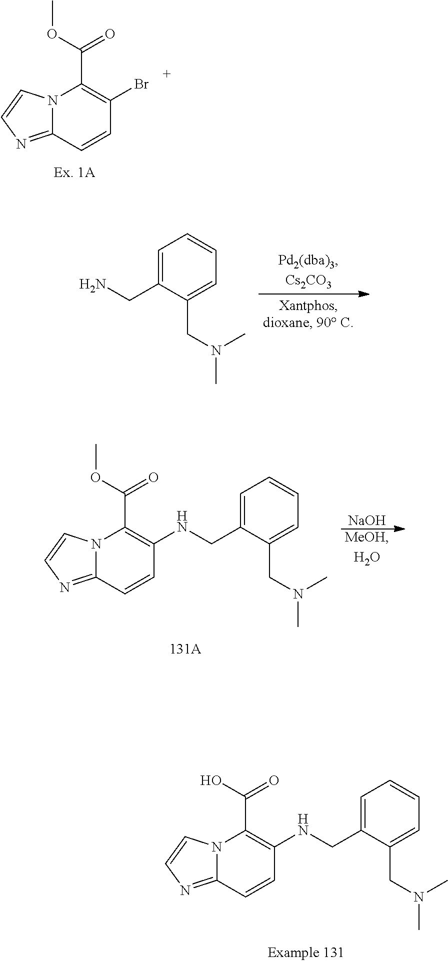

### Example 131A

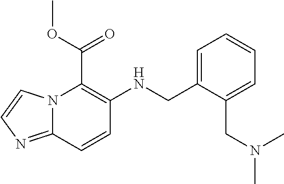 [SMILES: COC(=O)c1c(NCc2ccccc2CN(C)C)ccc2nccn12]

### Example 131A

To a solution of Example 1A (100 mg, 392 umol, 1 eq) and 1-(2-(aminomethyl)phenyl)-N,N-dimethylmethanamine (64.4 mg, 392 umol, 1 eq) in dioxane (1 mL) was added cesium carbonate (255.5 mg, 784 umol, 2 eq), Pd₂(dba)₃ (35.9 mg, 39.2 umol, 0.10 eq) and Xantphos (45.4 mg, 78.4 umol, 0.20 eq). The reaction mixture was stirred under nitrogen at 90° C. for 12 hrs. The reaction was cooled to 25° C. and filtered. The filtrate was concentrated in vacuo and the residue purified by prep-TLC to give the title compound (80.0 mg, 60.3%) as a brown solid. ESI m/z 339.1 [M+1]⁺.

### Example 131

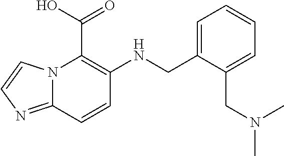 [SMILES: CN(C)Cc1ccccc1CNc1ccc2nccn2c1C(=O)O]

### Example 131

To a solution of Example 131A (80 mg, 236.4 umol, 1 eq) in methanol (5 mL) and water (1 mL) was added sodium hydroxide (18.9 mg, 472.8 umol, 2 eq). The reaction mixture was stirred at 70° C. for 1 h. The reaction was cooled to 25° C. and concentrated in vacuo. The residue was adjusted to pH 7 and filtered. The solid was collected and purified by prep-HPLC (base buffer) to give the title compound (22.7 mg, 49.6%) as a yellow solid. ¹H NMR: (DMSO-d6, 400 MHz): δ=10.94 (s, 1H), 8.87 (d, J=2.0 Hz, 1H), 8.12 (d, J=2.0 Hz, 1H), 7.96 (d, J=10.0 Hz, 1H), 7.81 (d, J=10.4 Hz, 1H), 7.73-7.67 (m, 1H), 7.43-7.38 (m, 2H), 7.34-7.30 (m, 1H), 5.02 (s, 2H), 4.43 (d, J=5.6 Hz, 2H), 2.78 (d, J=4.4 Hz, 6H). ESI m/z 325.2 [M+1]⁺.

### Scheme for the Synthesis of Example 132

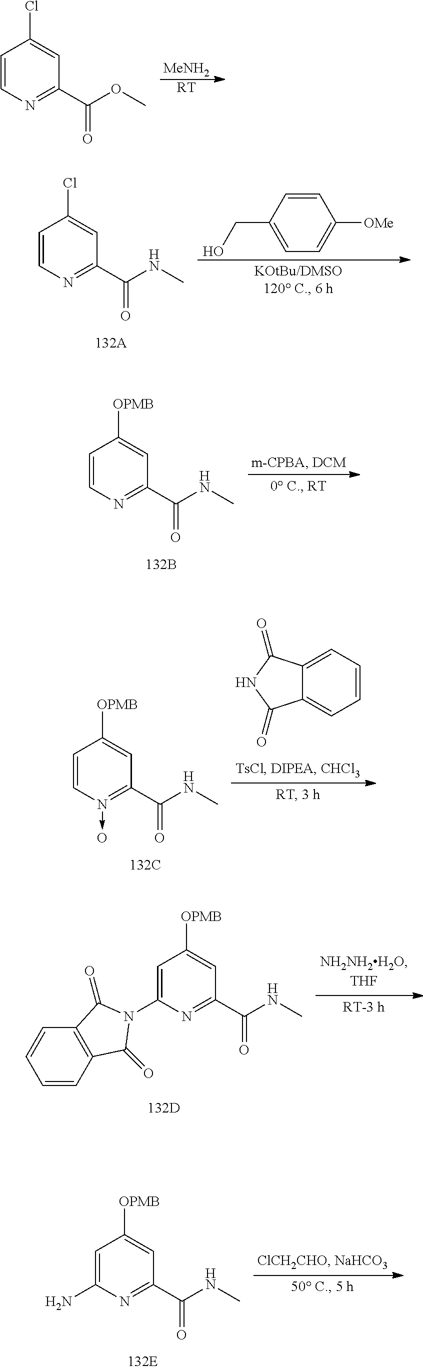

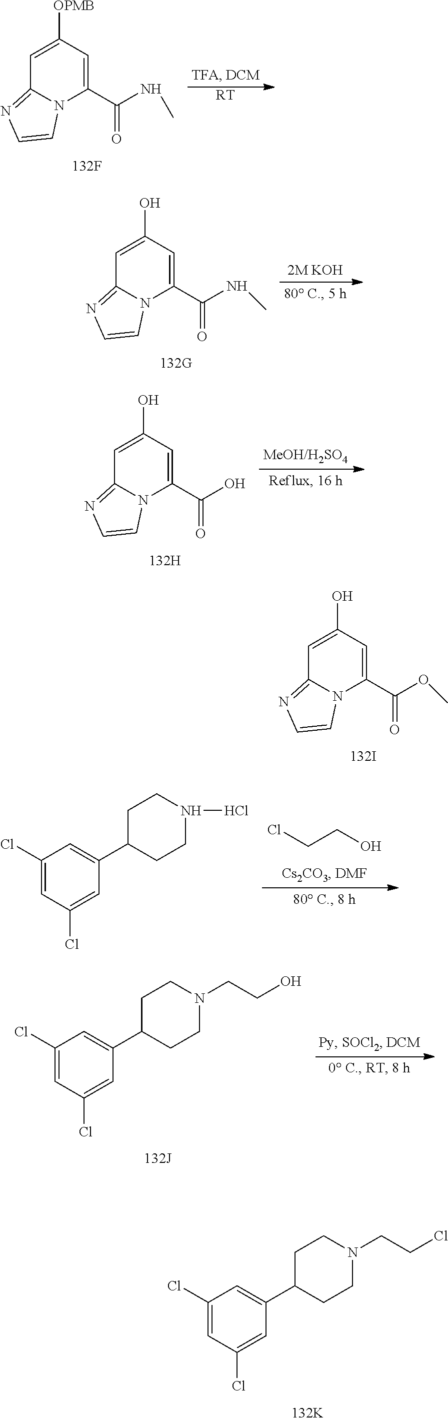

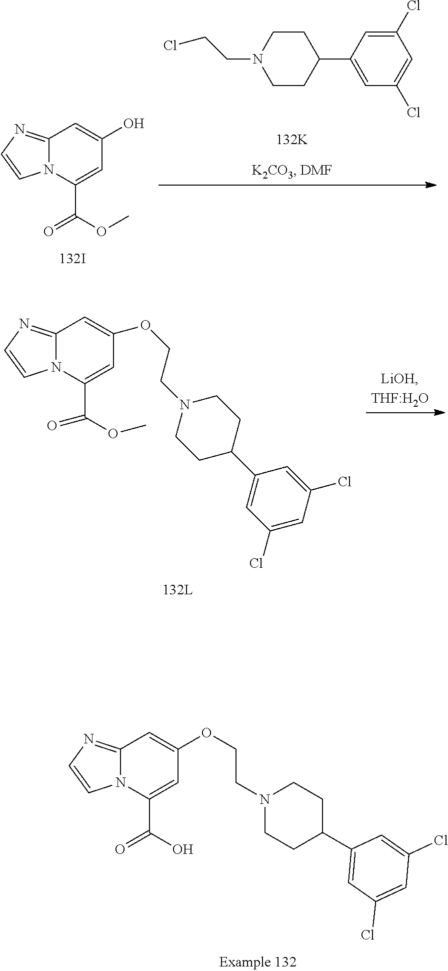 [SMILES: ClCCN1CCC(c2cc(Cl)cc(Cl)c2)CC1 ; COC(=O)c1cc(O)cc2nccn12 ; COC(=O)c1cc(OCCN2CCC(c3cc(Cl)cc(Cl)c3)CC2)cc2nccn12 ; O=C(O)c1cc(OCCN2CCC(c3cc(Cl)cc(Cl)c3)CC2)cc2nccn12]

### Example 132A

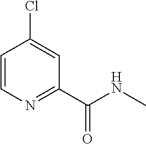

### Example 132A

A solution of methyl 4-chloropicolinate (3 g, 17.6 mmol) in 40% methyl amine solution (10 mL) was stirred for 3 h at room temperature. After completion of the reaction, water was added and the reaction mixture was extracted with ethyl acetate (2×50 mL). The combined organic layers were dried over anhydrous sodium sulfate and concentrated to provide crude material (2 g, 66%) which was used in the next step without purification. m/z: 171.6 [M+H]⁺.

### Example 132B

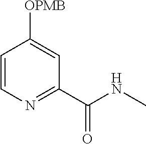

### Example 132B

To a stirred solution of Example 132A (4 g, 23.5 mmol) in dry DMSO (16 mL) was added 4-methoxybenzyl alcohol (4.3 mL, 47.7 mmol) and potassium tert-butoxide (2.6 g, 23.5 mmol). The resulting mixture was stirred at 120° C. for 5 h. After completion of the reaction, water was added and the mixture extracted with ethyl acetate (2×50 mL). The combined organic layers were dried over anhydrous sodium sulfate, concentrated under reduced pressure and purified by silica gel (100-200 mesh) column chromatography (gradient elution with 20% EtOAc in hexane) to afford the title compound (6 g, 93.7%) as an off-white solid. m/z: 273.2 [M+H]⁺.

### Example 132C

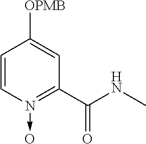

### Example 132C

To a stirred solution of Example 132B (6 g, 25.7 mmol) in dry DCM (50 mL) was added m-CPBA (13.3 g, 77.2 mmol) portion wise at 0° C. under inert atmosphere. The reaction was stirred at room temperature for 16 h. After completion of the reaction, the mixture was washed with 10% Na₂S₂O₃ solution and the resulting organic phase was washed with saturated NaHCO₃ solution. The organic phase was dried over anhydrous sodium sulfate, concentrated and purified by silica gel (100-200 mesh) column chromatography (gradient elution with MeOH in DCM) to afford the title compound (6 g, 95.2%) as a white solid. m/z: 289.5 [M+H]⁺.

### Example 132D

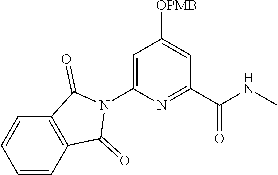

### Example 132D

To a stirred solution of Example 132C (6 g, 20.8 mmol) in CHCl₃ (30 mL) was added phthalimide (6.13 g, 41.2 mmol), p-toluenesulfonyl chloride (7.9 g, 41.2 mmol) and DIPEA (11.7 mL, 83.3 mmol). The resulting reaction was stirred at room temperature for 4 h. After completion of the reaction, water was added to reaction and the mixture extracted with DCM (2×50 mL). The combined organic layers were dried over anhydrous sodium sulfate, concentrated and purified by silica gel (60-120 mesh) column chromatography (gradient elution 30% EtOAC in hexane) to afford the title compound (95 g, 58.1%) as a white solid. m/z: 418.3 [M+H]⁺.

### Example 132E

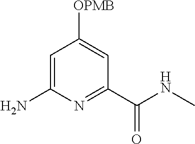

### Example 132E

A solution of Example 132D (2 g, 4.80 mmol) in a [1:1] mixture of THF: NH₂NH₂.H₂O (10 mL) was stirred for 2 h at room temperature. After completion of the reaction, water was added and the mixture was extracted with ethyl acetate (2×50 mL). The combined organic layers were dried over anhydrous sodium sulfate and concentrated to obtain the crude title compound, which was used in the next step without further purification. m/z: 288.2 [M+H]⁺.

### Example 132F

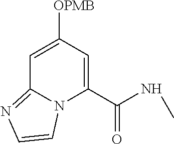

### Example 132F

To a stirred solution of Example 132E (1.5 g, 5.22 mmol) in ethanol (10 mL) were added 2-chloro acetaldehyde (7.7 mL, 120 mmol) and NaHCO₃ (746 mg, 8.88 mmol). The reaction was stirred at 50° C. for 5 h. After completion of the reaction, water was added and the mixture extracted with ethyl acetate (2×50 mL). The combined organic layers were dried over anhydrous sodium sulfate, concentrated and purified by alumina (neutral) column chromatography (gradient elution with MeOH in DCM) to afford the title compound (750 mg, 46%) as a white solid. m/z: 312.2 [M+H]⁺.

### Example 132G

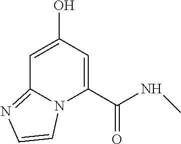

### Example 132G

To a stirred solution of Example 132F (500 mg, 1.60 mmol) in DCM (20 mL) was added TFA (2 mL, 5.20 mmol) at 0° C. and stirring was continued at room temperature for 2 h. After completion of the reaction, water was added and the reaction mixture was extracted with DCM (2×50 mL). The combined organic layers were dried over anhydrous sodium sulfate, concentrated and purified by silica gel (60-120 mesh) column chromatography (gradient elution 5% MeOH in DCM) to afford the title compound (160 mg, 52%) as an off-white semi solid. m/z: 192.2 [M+H]⁺.

### Example 132H

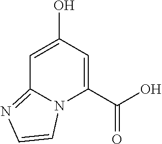 [SMILES: O=C(O)c1cc(O)cc2nccn12]

### Example 132H

Example 132G (800 mg, 4.18 mmol) suspended in 2M KOH (6 mL) was heated at 80° C. for 5 h. After cooling, the reaction mixture was concentrated under reduced pressure and the residue acidified to pH 2 with 2M HCl. The resulting aqueous phase was again concentrated to obtain a crude solid, which was dissolved in methanol and filtered. The filtrate was concentrated to obtain the crude title compound which was used in the next step without further purification. m/z: 179.1 [M+H]⁺.

### Example 132I

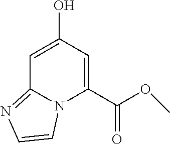

### Example 132I

To a stirred solution of Example 13211 (600 mg, 0.73 mmol) in methanol (10 mL) was added conc. H2SO₄ (0.5 mL). The reaction mixture was heated at reflux for 16 h. After cooling, the reaction mixture was concentrated, basified with saturated NaHCO₃ and extracted with ethyl acetate (2×50 mL). The combined organic layers were dried over anhydrous sodium sulfate and concentrated to afford the title compound (440 mg, 68%) as an off-white solid. m/z: 193.2 [M+H]⁺.

### Example 132J

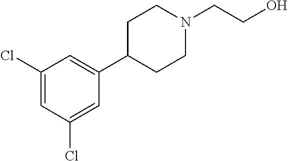 [SMILES: OCCN1CCC(c2cc(Cl)cc(Cl)c2)CC1]

### Example 132J

To a stirred solution of 4-(3,5-dichlorophenyl)piperidine hydrochloride (200 mg, 0.869 mmol) in dry DMF (4 mL) were added Cs₂CO₃ (170 mg, 1.739 mmol) and 2-chloroethanol (0.02 mL, 1.304 mmol) at room temperature. The resulting reaction mixture was stirred at 80° C. for 8 h. After cooling, the mixture was diluted with water and extracted with ethyl acetate (2×20 mL). The combined organic layers were dried over anhydrous sodium sulfate, concentrated and purified by silica gel (100-200 mesh) column chromatography (gradient elution with 1% MeOH in DCM) to afford the title compound (100 mg, 42%) as an off white solid. m/z: 274.19 [M+H]⁺.

### Example 132K

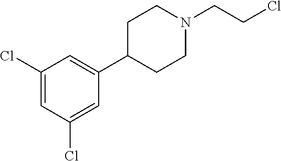 [SMILES: ClCCN1CCC(c2cc(Cl)cc(Cl)c2)CC1]

### Example 132K

To a stirred solution of Example 132J (50 mg, 0.182 mmol) in dry DCM (2 mL) were added pyridine (0.07 mL, 0.55 mmol) and SOCl₂ (0.03 mL, 0.273 mmol) at 0° C. The reaction was stirred at room temperature for 16 h. The reaction mixture was concentrated and the residue was acidified to pH ˜2 with 2N HCl aqueous solution. The mixture was extracted with DCM (2×5 mL). The combined organic layers were concentrated and purified by silica gel (100-200 mesh) column chromatography (gradient elution with 1% MeOH in DCM) to afford the title compound (40 mg, 75%) as an off-white semi solid. m/z: 292.2 [M+H]⁺.

### Example 132L

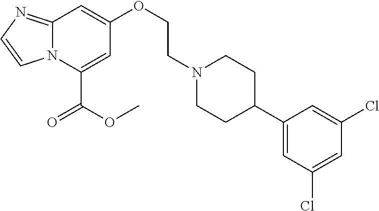 [SMILES: COC(=O)c1cc(OCCN2CCC(c3cc(Cl)cc(Cl)c3)CC2)cc2nccn12]

### Example 132L

To a solution of Example 132I (200 mg, 0.68 mmol) in dry DMF (5 mL) in a microwave tube were added K₂CO₃ (189 mg, 1.37 mmol) and Example 132K (233 mg, 1.02 mmol). The reaction was irradiated at 120° C. for 60 min. After cooling, ice-water (20 mL) was added and the mixture was extracted with ethyl acetate (2×20 mL). The combined organic layers were dried over anhydrous sodium sulfate, concentrated and purified by alumina (basic) column chromatography (gradient elution with 1% MeOH in DCM) to afford the title compound (190 mg, 62%) as a brown semi solid. m/z: 448.3 [M+H]⁺.

### Example 132

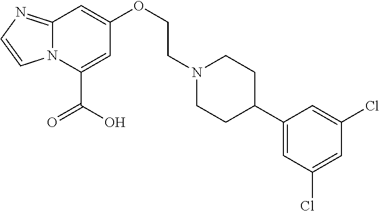 [SMILES: O=C(O)c1cc(OCCN2CCC(c3cc(Cl)cc(Cl)c3)CC2)cc2nccn12]

### Example 132

To a stirred solution of Example 132L (190 mg, 0.424 mmol) in [4:1] solvent mixture of THF (4 mL) and H₂O (1.0 mL) was added LiOH.H₂O (89.5 mg, 2.12 mmol) at 0° C. The reaction was stirred at room temperature for 2 h. The reaction mixture was concentrated to remove THF and acidified to pH 2 with 1M HCl solution. The resulting aqueous phase was purified by preparative HPLC to afford the TFA salt of the title compound (15 mg, 13.6%) as a white solid. m/z: 434.3 [M+H]⁺.

¹H NMR (300 MHz, DMSO-d6): δ 8.91 (1H, s), 7.98 (1H, s), 7.71 (1H, s), 7.49-7.45 (2H, m), 7.36-7.31 (2H, m), 4.61 (2H, J=4.42 Hz, t), 3.25-3.16 (2H, m), 2.98-2.91 (2H, m), 2.11-2.06 (2H, m), 2.01-1.88 (3H, m).

### Scheme for the Synthesis of Example 133

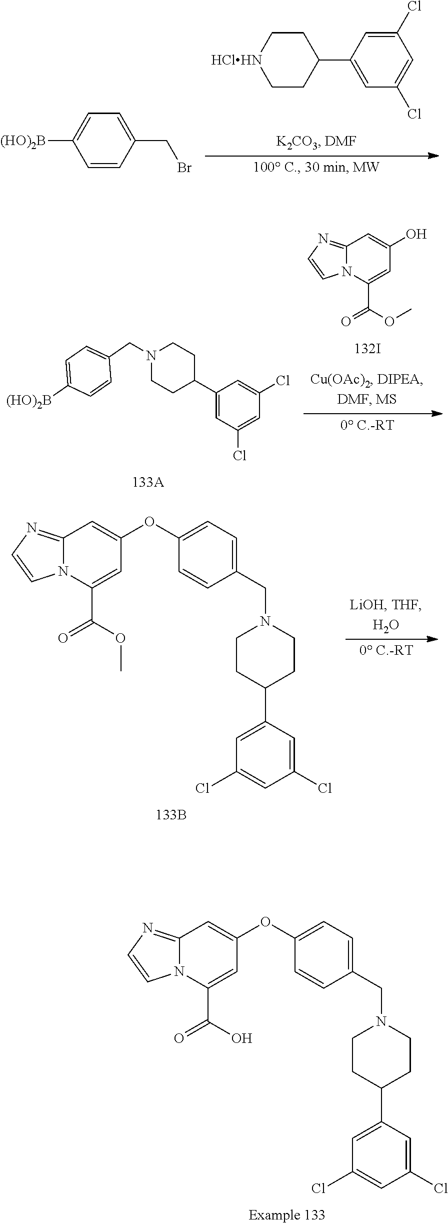

### Example 133A

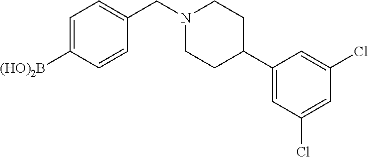 [SMILES: OB(O)c1ccc(CN2CCC(c3cc(Cl)cc(Cl)c3)CC2)cc1]

### Example 133A

To a stirred solution of (4-(bromomethyl)phenyl)boronic acid (200 mg, 1.12 mmol) in dry DMF (3 mL) in a microwave tube were added 4-(3,5-dichlorophenyl)piperidine hydrochloride (214 mg, 0.93 mmol) and K₂CO₃ (257 mg, 1.86 mmol) under an inert atmosphere. The microwave tube was irradiated at 100° C. for 30 min. After cooling, the reaction mixture was diluted with ice water (15 mL) and filtered. The collected solid was purified by silica gel (60-120 mesh) column chromatography (gradient elution with 3% MeOH in DCM) to afford the title compound (100 mg, 30%) as an off-white solid. m/z: 364.3 [M+H]⁺.

### Example 133B

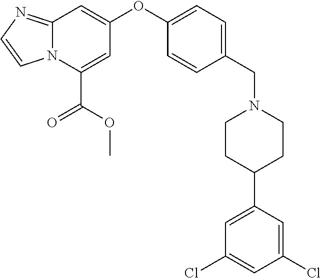 [SMILES: COC(=O)c1cc(Oc2ccc(CN3CCC(c4cc(Cl)cc(Cl)c4)CC3)cc2)cc2nccn12]

### Example 133B

To a stirred solution of Example 132I (250 mg, 1.29 mmol) in a [4:1] solvent mixture of dry DCM (10 mL) and DMF (2.5 mL) were added Example 133A (700 mg, 1.94 mmol), DIPEA (1.19 mL, 6.5 mmol), Cu(OAc)₂ (518 mg, 2.78 mmol) and molecular sieves 4 Å (250 mg). The reaction was stirred at room temperature for 16 h. The reaction mixture was filtered through a celite pad and the pad washed with DCM. The collected filtrate was concentrated and purified by silica gel (60-120 mesh) column chromatography (gradient elution with 5% MeOH in DCM) to afford the title compound (200 mg, 30%) as an off-white solid. m/z: 510.4 [M+H]⁺.

### Example 133

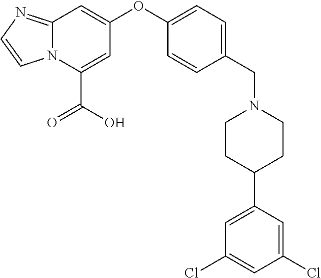 [SMILES: O=C(O)c1cc(Oc2ccc(CN3CCC(c4cc(Cl)cc(Cl)c4)CC3)cc2)cc2nccn12]

### Example 133

To a stirred solution of Example 133B (90 mg, 0.17 mmol) in a [4:1] solvent mixture of THF (2 mL) and H₂O (0.5 mL) was added LiOH.H₂O (41 mg, 0.88 mmol) at 0° C. The reaction was stirred for 1 h at room temperature. The reaction mixture was concentrated to remove THF and acidified to pH 2 with 1M HCl solution. The resulting aqueous phase was purified by preparative HPLC and lyophilized to afford the TFA salt of the title compound (5.2 mg, 6%) as a light brown semi solid. m/z: 496.3 [M+H]⁺.

¹H NMR (300 MHz, CD₃OD): δ 9.10 (1H, s), 7.93 (1H, s), 7.83-7.62 (5H, m), 7.28-7.14 (3H, m), 6.97 (1H, s), 4.43 (2H, s), 3.65-3.51 (2H, m), 3.17-3.08 (2H, m), 2.92-2.79 (1H, m), 2.11-1.83 (4H, m).

### Scheme for the Synthesis of Example 134

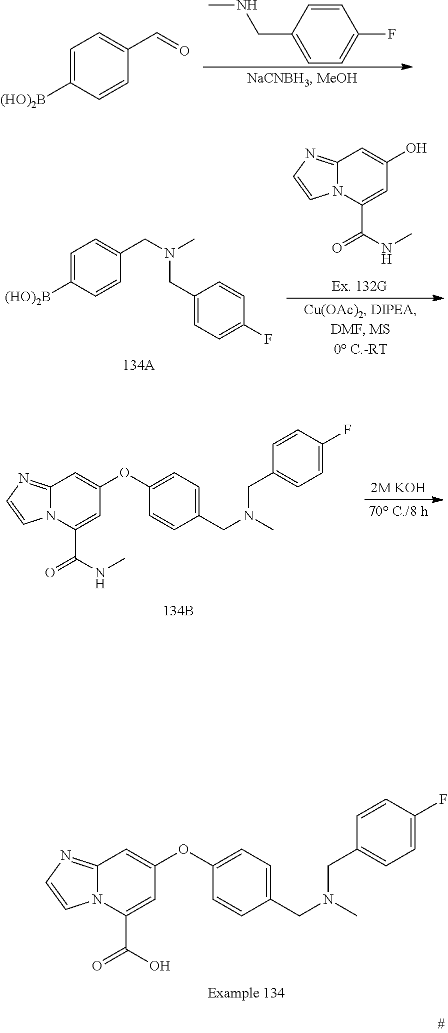

### Example 134A

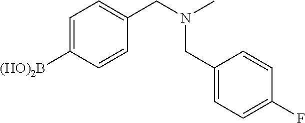 [SMILES: CN(Cc1ccc(F)cc1)Cc1ccc(B(O)O)cc1]

### Example 134A

To a stirred solution of (4-formylphenyl)boronic acid (50 mg, 0.33 mmol) in dry MeOH (3 mL) were added 1-(4-fluorophenyl)-N-methylmethanamine (55 mg, 0.4 mmol) and acetic acid (20 mg, 0.33 mmol) at 0° C. The reaction was stirred for 30 min and then NaCNBH₃ (41.4 mg, 0.66 mmol) was added at the same temperature. The resultant reaction mixture was stirred at room temperature for 16 h. After completion of the reaction, the reaction mixture was diluted with ice cold water and extracted with EtOAc (2×20 mL). The combined organic layers were dried over anhydrous sodium sulfate, concentrated and purified by silica gel (60-120 mesh) column chromatography (gradient elution with 2% MeOH in DCM) to afford the title compound (20 mg, 21%) as an off-white solid. m/z: 364.3 [M+H]⁺.

### Example 134B

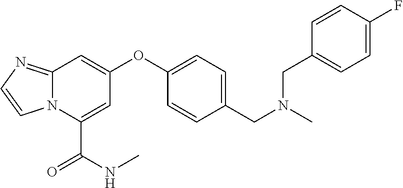 [SMILES: CNC(=O)c1cc(Oc2ccc(CN(C)Cc3ccc(F)cc3)cc2)cc2nccn12]

### Example 134B

To a stirred solution of Example 132G (160 mg, 0.83 mmol) in [4:1] solvent mixture of dry DCM (10 mL) and DMF (2.5 mL) were added Example 134A (274 mg, 1.0 mmol), DIPEA (0.764 mL, 4.5 mmol), Cu(OAc)₂ (331 mg, 1.66 mmol) and molecular sieves 4 Å (200 mg) at 0° C. The reaction was stirred for 16 h at room temperature. The reaction mixture was filtered through celite pad and the filtrate was concentrated. The residue was purified by silica gel (60-120 mesh) column chromatography (gradient elution with 10% MeOH in DCM) to afford the title compound (120 mg, 28%) as an off-white solid. m/z: 419.2 [M+H]⁺.

### Example 134

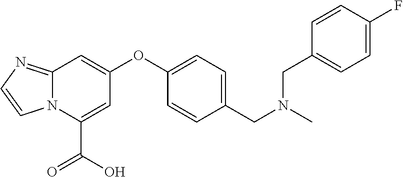 [SMILES: CN(Cc1ccc(F)cc1)Cc1ccc(Oc2cc(C(=O)O)n3ccnc3c2)cc1]

### Example 134

Example 134B (80 mg, 0.191 mmol) suspended in 2M KOH (3 mL) was heated at 80° C. for 8 h. After cooling, the reaction mixture was concentrated under reduced pressure and acidified to pH 2 with 1M HCl. The resulting aqueous phase was purified by preparative HPLC to afford the TFA salt of the title compound (8 mg, 14.5%) as an off-white solid. m/z: 406.3 [M+H]⁺.

¹H NMR (300 MHz, CD₃OD): δ 9.30 (1H, s), 8.00 (1H, s), 7.84-7.79 (5H, m), 7.61-7.56 (2H, m), 7.27-7.22 (2H, m), 7.05 (1H, s), 4.49 (2H, s), 4.42 (2H, s), 2.75 (3H, s).

### Scheme for the Synthesis of Example 135

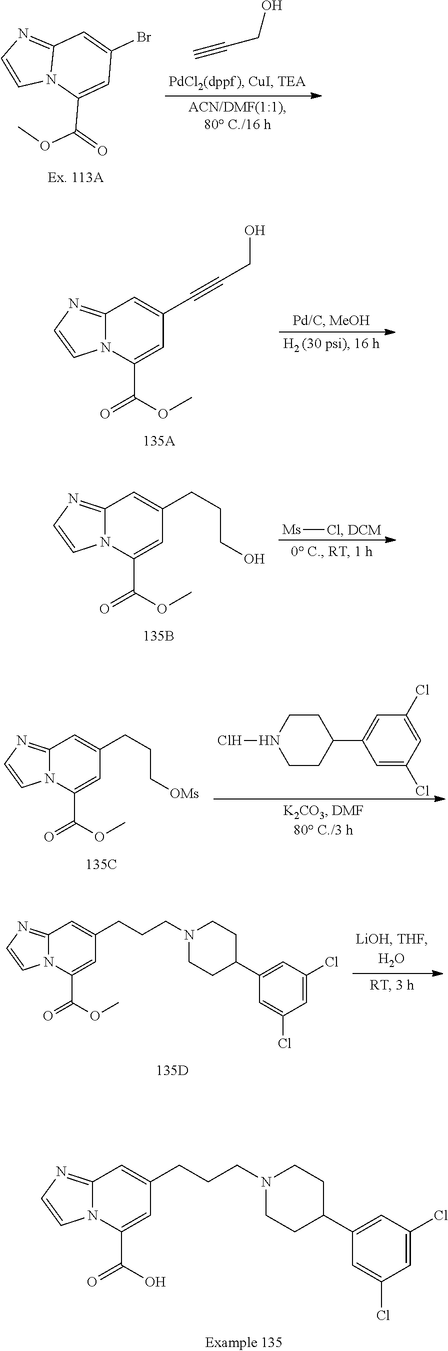

### Example 135A

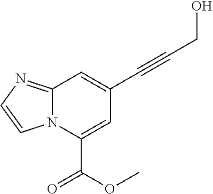 [SMILES: COC(=O)c1cc(C#CCO)cc2nccn12]

### Example 135A

To a stirred solution of Example 113A (200 mg, 0.78 mmol) in a [1:1] solvent mixture of ACN: DMF (2 mL) were added propargyl alcohol (0.6 mL, 2.35 mmol), CuI (7 mg, 0.039 mmol) and TEA (1 mL). The reaction mixture was degassed with nitrogen for 20 min and PdCl₂(dppf) (5 mg, 0.078 mmol) was added at room temperature. The reaction mixture was heated at 80° C. for 16 h. After cooling, the reaction mixture was filtered through a celite pad and the pad washed with DCM (20 mL). The filtrate was concentrated and purified by alumina (basic) column chromatography (gradient elution with 2% MeOH in DCM) to afford the title compound (75 mg, 41%) as brown solid. m/z: 231.1[M+H]⁺.

### Example 135B

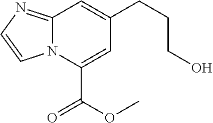 [SMILES: COC(=O)c1cc(CCCO)cc2nccn12]

### Example 135B

To a stirred solution of Example 135A (50 mg, 0.217 mmol) in MeOH (3 mL) and acetic acid (0.1 mL) was added Pd/C (25 mg) under an inert atmosphere. The reaction mixture was stirred under 30 psi hydrogen pressure for 16 h. The reaction mixture was filtered through a celite pad and the pad washed with MeOH. The filtrate was concentrated to afford the title compound (40 mg, 80%) as a brown solid. m/z: 235.1 [M+H]⁺.

### Example 135C

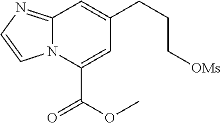 [SMILES: COC(=O)c1cc(CCCOS(C)(=O)=O)cc2nccn12]

### Example 135C

To a stirred solution of Example 135B (90 mg, 0.38 mmol) in DCM (5 mL) at 0° C. were added drop wise triethylamine (0.161 mL, 1.15 mmol) and methane sulfonyl chloride (0.086 mL, 0.76 mmol). The reaction was stirred at the same temperature for 1 h. The reaction was quenched with water (10 mL) and extracted with DCM (2×10 mL). The combined organic layers were dried over anhydrous sodium sulfate and concentrated under reduced pressure to afford the title compound (30 mg, 55%) as a pale yellow semi-solid.

### Example 135D

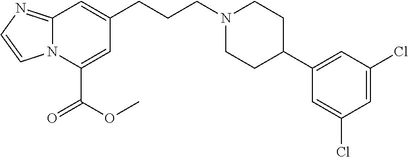 [SMILES: COC(=O)c1cc(CCCN2CCC(c3cc(Cl)cc(Cl)c3)CC2)cc2nccn12]

### Example 135D

To a stirred solution of 4-(3,5-dichlorophenyl)piperidine hydrochloride (87 mg, 0.38 mmol) in dry DMF (3 mL) was added potassium carbonate (132 mg, 0.96 mmol) followed by Example 135C (100 mg, 0.32 mmol) under an inert atmosphere. The reaction was stirred at 80° C. for 3 h. The reaction mixture was cooled to room temperature and filtered. The filtrate was concentrated and purified by alumina (basic) column chromatography (gradient elution with 1% MeOH in DCM) to afford the title compound (50 mg, 40%) as a pale yellow solid. m/z: 446.13 [M+H]⁺.

### Example 135

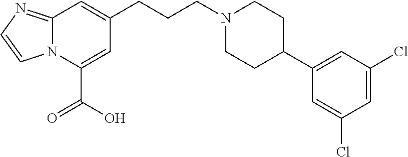 [SMILES: O=C(O)c1cc(CCCN2CCC(c3cc(Cl)cc(Cl)c3)CC2)cc2nccn12]

### Example 135

To a stirred solution of Example 135D (50 mg, 0.112 mmol) in a [4:1] solvent mixture of THF (2 mL) and H₂O (0.5 mL) was added LiOH.H₂O (20 mg, 0.22 mmol) at 0° C. The reaction was stirred at room temperature for 1 h. The reaction mixture was concentrated to remove THF and acidified to pH 2 with 1M HCl. The resulting aqueous phase was purified by preparative HPLC to afford the TFA salt of the title compound (5.1 mg, 10.5%) as a brown semi-solid. m/z: 432.3 [M+H]⁺.

¹H NMR (300 MHz, CD₃OD): δ 9.07 (1H, s), 7.86 (1H, s), 7.72 (1H, s), 7.24 (1H, s), 7.17 (1H, s), 3.68-3.53 (2H, m), 3.10-2.81 (7H, m), 2.25-1.81 (6H, m).

### Example 136

This Example illustrates that the exemplary compounds disclosed herein inhibit KDM5B enzymatic activity.

Ten point dose-response curves for the compounds of Examples 1-135 were determined using a homogeneous time resolved fluorescence (HTRF) assay (Reaction Biology Corp, Malvern, Pa.). This assay combines fluorescence resonance energy transfer (FRET) with time resolve (TR) measurements (TR-FRET). The KDM5B α-ketoglutarate-dependent demethylase activity is calculated by measuring demethylation of a biotin labeled histone tri-methylated H3K4 substrate using an europium-cryptate (Eu)-labeled antibody donor specific for dimethylated histone H3K4 and a Streptavidin-XL665 acceptor that binds to the biotin group of substrate, and detecting FRET by exciting the reaction mixture at 320 nm and reading dual emissions at 615 nm and 665 nm.

Briefly, compounds disclosed herein were solubilized in DMSO and a series of 10, three-fold serial dilutions were made for each compound in 100% DMSO. The initial starting concentration for the serial dilutions of each compound was 10 μM or 100 μM. Control samples lacking compound, KDM5B enzyme or various reaction components also were prepared and processed in parallel with compound test samples.

An aliquot of each serial dilution of test compound was added to a 384 well plate (Corning Cat #3572) containing 1.2 nM KDM5B enzyme suspended in 50 mM Hepes, pH7.5, 50 mM NaCl, 0.01% Tween 20, 0.1% BSA, and 1% DMSO (final concentrations) in a 10 microliter reaction volume using a LABCYTE ECHO liquid handler. The samples were mixed, subjected to centrifugation and the plate was pre-incubated at room temperature for 15 minutes (min), to which 30 nM Biotin-H3K4Me3 1-21 substrate (Anaspec, Freemont Calif., Cat #64192), 20 μM Fe(II) and 20 μM α-ketoglutarate co-factors, and 100 μM Ascorbate were added to initiate the enzymatic reaction. The reaction mixture was incubated at room temperature for 45 minutes. A 10 μl aliquot of a detection mixture of Eu-labeled anti-histone H3K4Me2 antibody (CisBIO, Bedford Mass., Cat #610AXLB) and Streptavidin-XL665 (CisBIO, Bedford Mass., Cat #610 SAXLB) in 200 mM potassium fluoride and 10 mM EDTA was added and kinetic measurements were read at 5 minute intervals for a period of 30 minutes using an Envision Multiplate Reader (PerkinElmer Model 2102; excitation at 320 nm and emission reads at 615 nm and 665 nm). The IC₅₀ value for each compound was determined from the 665/615 ratio obtained for each 10 point dose-response curve using GraphPad Prism 4 software with a sigmodial dose response.

The results for exemplary compounds of Formula (I) are shown in Table 2.

Key: “A”≤500 nM; “B”>500 nM−≤1 μM; “C”>1 μM; NT, not tested.

TABLE 2
Inhibition of KDM5B Activity by Exemplary
Compounds of Formula (I)

| EXAMPLE Number | IC50 (μM) |
|---|---|
| 1 | A |
| 2 | B |
| 3 | B |
| 4 | A |
| 5 | C |
| 6 | C |
| 7 | C |
| 8 | A |
| 9 | C |
| 10 | C |
| 11 | C |
| 12 | B |
| 13 | A |
| 14 | C |
| 15 | B |
| 16 | C |
| 17 | C |
| 18 | C |
| 19 | C |
| 20 | C |
| 21 | A |
| 22 | C |
| 23 | A |
| 24 | C |
| 25 | C |
| 26 | B |
| 27 | C |
| 28 | C |
| 29 | B |
| 30 | C |
| 31 | C |
| 32 | A |
| 33 | C |
| 34 | B |
| 35 | C |
| 36 | C |
| 37 | B |
| 38 | C |
| 39 | C |
| 40 | C |
| 41 | B |
| 42 | C |
| 43 | C |
| 44 | B |
| 45 | A |
| 46 | C |
| 47 | B |
| 48 | C |
| 49 | C |
| 50 | C |
| 51 | A |
| 52 | C |
| 53 | C |
| 54 | C |
| 55 | B |
| 56 | C |
| 57 | B |
| 58 | C |
| 59 | A |
| 60 | A |
| 61 | B |
| 62 | B |
| 63 | C |
| 64 | C |
| 65 | C |
| 66 | C |
| 67 | A |
| 68 | C |
| 69 | C |
| 70 | C |
| 71 | B |
| 72 | B |
| 73 | C |
| 74 | C |
| 75 | C |
| 76 | B |
| 77 | B |
| 78 | A |
| 79 | C |
| 80 | C |
| 81 | B |
| 82 | B |
| 83 | A |
| 84 | A |
| 85 | C |
| 86 | C |
| 87 | C |
| 88 | A |
| 89 | C |
| 90 | C |
| 91 | C |
| 92 | B |
| 93 | C |
| 94 | A |
| 95 | C |
| 96 | C |
| 97 | B |
| 98 | C |
| 99 | C |
| 100 | B |
| 101 | C |
| 102 | C |
| 103 | C |
| 104 | C |
| 105 | C |
| 106 | C |
| 107 | C |
| 108 | C |
| 109 | C |
| 110 | C |
| 111 | C |
| 112 | C |
| 113 | A |
| 114 | C |
| 115 | C |
| 116 | C |
| 117 | C |
| 118 | C |
| 119 | C |
| 120 | C |
| 121 | C |
| 122 | C |
| 123 | C |
| 124 | C |
| 125 | C |
| 126 | B |
| 127 | A |
| 128 | C |
| 129 | A |
| 130 | B |
| 131 | C |
| 132 | C |
| 133 | NT |
| 134 | C |
| 135 | A |

### Example 137

This Example illustrates a methodology for testing compounds disclosed herein for inhibition of growth of tumor cell lines that express KDM5B.

The ZR-75-1 cell line cell line was established from the mammary gland of a 63-year-old human female, derived from a metastatic site, and has been shown to be sensitive to inhibitors of KDM5B.

Inhibition of KDM5B-mediated cellular proliferation by compounds of Formula (I) is measured in a CellTiter Glo luminescence assay (Promega Corp, Madison, Wis.), which determines the number of viable cells by quantitating the amount of ATP, using a BMG LabTech CLARIOStar instrument in accordance with the manufacturer's instructions. Briefly, ZR-75-1 cells are plated at a density of 1500 cells/90 μl/well in 96 well culture plates and cultured in RPMI 1640 medium (Gibco) supplemented with 10% fetal bovine serum (FBS) and 1% penicillin and 1% streptomycin at 37° C. A series of 3-fold serial dilutions of each test compound of Formula (I) is prepared in complete RPMI 1640 medium and added to the cells at final concentrations ranging from 10 μM to 0.0015 nM. Control samples lacking test compound or cells are processed in parallel. The plates are incubated at 37° C. for four days and thereafter 50 μl fresh medium containing the same concentration of test compound is added. The plates are incubated for an additional three days (Day 7), at which time 50 uL is removed from each well and replaced with 50 uL fresh medium containing the same concentration of test compound, and plates are incubated for an additional three days (Day 10). A baseline measurement, as described below, is taken for a time zero point at Day 0.

At Day 10, the supernatant is removed by aspiration and the plate is allowed to equilibrate to room temperature (˜15 min). The cells are lysed using 30 μl (30 μl for Day 0) of Cell Titer Glo reagent (Promega Corp, Madison, Wis.). The plates are shaken for two minutes and incubated at room temperature for 30 minutes protected from light. The degree of inhibition of cell viability is determined using a spectrophotometric readout by measuring the luminescence at 340 nm and the EC₅₀ concentration for each compound is calculated using Graph Pad Prism 4 software.

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A compound of formula (I): 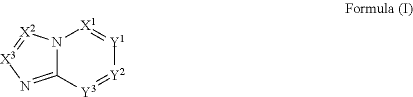
or a pharmaceutical salt thereof:
wherein:
X¹ and X² are each independently N, CR¹, or CR³, wherein at least one of X¹ or X² is CR¹;
X³ is N or CR³;
Y¹ and Y² are each independently N, CR² or CR³, wherein at least one of Y¹ or Y² is CR² and R² is -L-R⁵;
Y³ is N or CR³;
R¹ is hydroxyl, cyano, —COOR⁴, —C(O)N(H)CN, —C(O)N(H)OH, or tetrazolyl;
R² is hydrogen, halogen, alkyl alkoxy, or -L-R⁵;
L is a bond or —(CH₂)ₘ—W—(CH₂)ₙ—;
W is absent, NR⁴, O, C(O), C(O)NR⁴; NR⁴C(O), S, SO, SO₂, NR⁴SO₂ or SO₂NR⁴;
R³ is hydrogen, halogen, alkyl or alkoxy;
R⁴ is hydrogen or alkyl;
R⁵ is hydrogen, carbocyclyl, heterocyclyl, aryl, or heteroaryl, wherein each of the carbocyclyl, heterocyclyl, aryl, or heteroaryl may be optionally substituted with one or more R⁶;
R⁶ is selected from the group consisting of hydroxyl, hydroxylalkyl, alkyl, arylalkyl, alkyl sulfonyl, halogen, haloalkyl, alkoxy, haloalkylalkoxy, cyano, acylaminyl, dialkylaminyl, aryl, aryloxy, alkoxyaryl, arylhydroxyalkyl, heterocyclyl, alkylheterocyclyl and heteroaryl; and
m and n are each independently zero or an integer between one and three.

2. The compound of claim 1, wherein X¹ is CR¹.

3. The compound of claim 1, wherein X¹ is CR¹.

4. The compound of claim 3, wherein R¹ is tetrazolyl.

5. The compound of claim 3, wherein R¹ is —COOR⁴.

6. The compound of claim 5, wherein R⁴ is hydrogen.

7. The compound of claim 1, wherein L is —NR⁴CH₂—.

8. The compound of claim 7, wherein R⁵ is aryl.

9. The compound of claim 8, wherein the aryl is selected from the group consisting of phenyl, naphthyl and tetrahydronaphthyl.

10. The compound of claim 9, wherein the aryl is phenyl.

11. The compound of claim 10, wherein the phenyl is substituted with one or more R⁶.

12. The compound of claim 10, wherein R⁶ is selected from the group consisting of hydroxyl, hydroxylalkyl, alkyl, arylalkyl, alkylsulfonyl, halogen, haloalkyl, alkoxy, haloalkylalkoxy, cyano, acylaminyl, dialkylaminyl, aryl, aryloxy, alkoxyaryl, arylhydroxyalkyl, heterocyclyl, alkylheterocyclyl and heteroaryl.

13. The compound of claim 7, wherein R⁵ is heteroaryl.

14. The compound of claim 13, wherein the heteroaryl is benzofuranyl, benzothophenyl, benzimidazolone, dihydrobenzodioxinyl and dihydroisoquinolinyl.

15. The compound of claim 14, wherein each of the benzofuranyl, benzothophenyl, benzimidazolone, dihydrobenzodioxinyl and dihydroisoquinolinyl is substituted with one or more R⁶.

16. The compound of claim 15, wherein R⁶ is selected from the group consisting alkyl and halogen.

17. The compound of claim 1, wherein the compound is selected from the group consisting of:

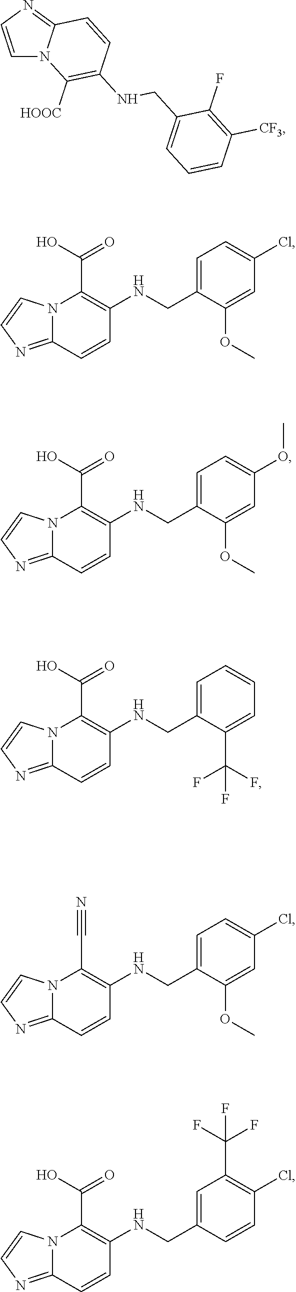 [SMILES: O=C(O)c1c(NCc2cccc(C(F)(F)F)c2F)ccc2nccn12 ; COc1cc(Cl)ccc1CNc1ccc2nccn2c1C(=O)O ; COc1ccc(CNc2ccc3nccn3c2C(=O)O)c(OC)c1 ; O=C(O)c1c(NCc2ccccc2C(F)(F)F)ccc2nccn12 ; COc1cc(Cl)ccc1CNc1ccc2nccn2c1C#N ; O=C(O)c1c(NCc2ccc(Cl)c(C(F)(F)F)c2)ccc2nccn12]

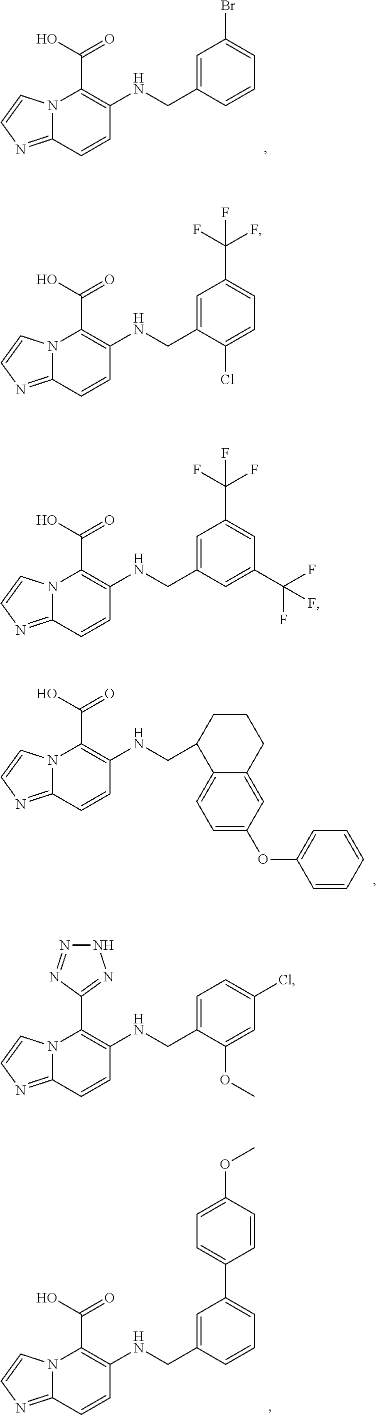 [SMILES: O=C(O)c1c(NCc2cccc(Br)c2)ccc2nccn12 ; O=C(O)c1c(NCc2cc(C(F)(F)F)ccc2Cl)ccc2nccn12 ; O=C(O)c1c(NCc2cc(C(F)(F)F)cc(C(F)(F)F)c2)ccc2nccn12 ; O=C(O)c1c(NCC2CCCc3cc(Oc4ccccc4)ccc32)ccc2nccn12 ; COc1cc(Cl)ccc1CNc1ccc2nccn2c1-c1nn[nH]n1 ; COc1ccc(-c2cccc(CNc3ccc4nccn4c3C(=O)O)c2)cc1]

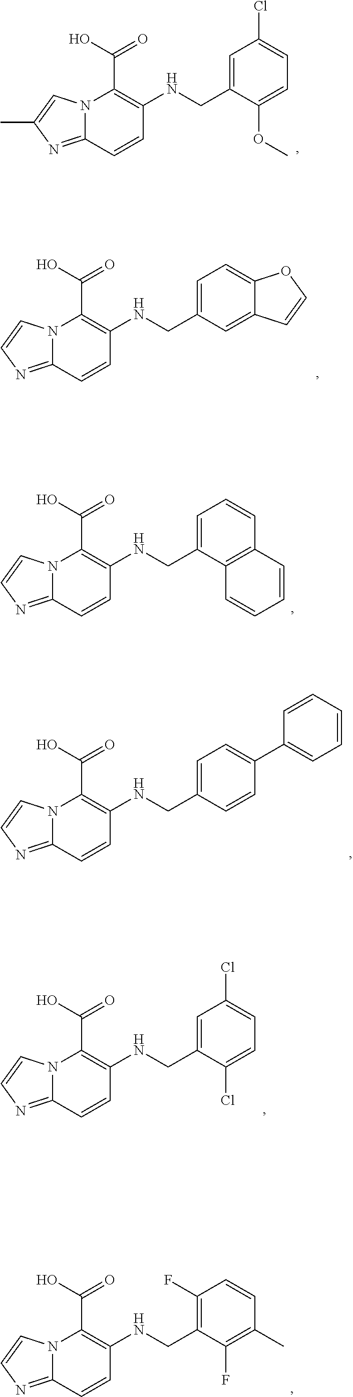

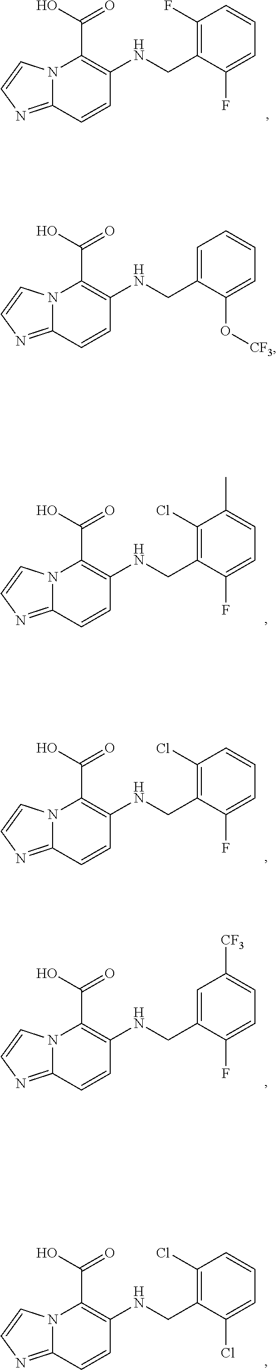 [SMILES: O=C(O)c1c(NCc2c(F)cccc2F)ccc2nccn12 ; O=C(O)c1c(NCc2ccccc2OC(F)(F)F)ccc2nccn12 ; Cc1ccc(F)c(CNc2ccc3nccn3c2C(=O)O)c1Cl ; O=C(O)c1c(NCc2c(F)cccc2Cl)ccc2nccn12 ; O=C(O)c1c(NCc2cc(C(F)(F)F)ccc2F)ccc2nccn12 ; O=C(O)c1c(NCc2c(Cl)cccc2Cl)ccc2nccn12]

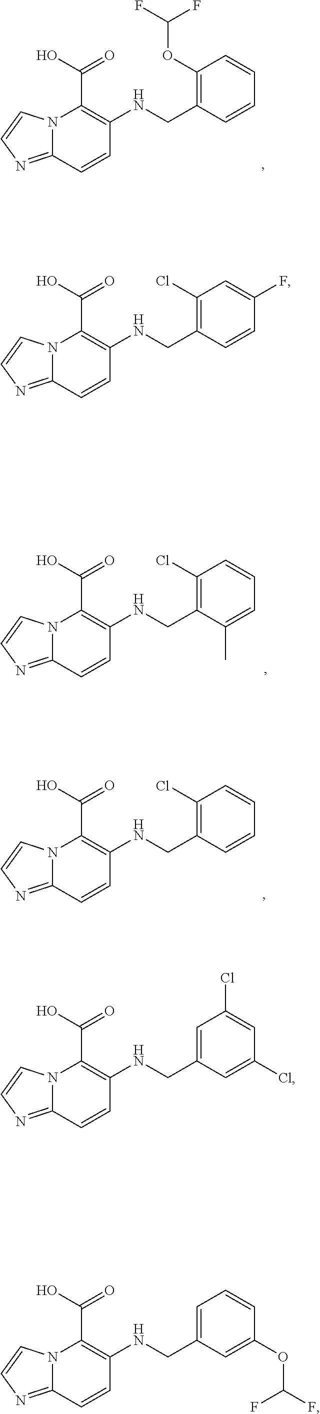 [SMILES: O=C(O)c1c(NCc2ccccc2OC(F)F)ccc2nccn12 ; O=C(O)c1c(NCc2ccc(F)cc2Cl)ccc2nccn12 ; Cc1cccc(Cl)c1CNc1ccc2nccn2c1C(=O)O ; O=C(O)c1c(NCc2ccccc2Cl)ccc2nccn12 ; O=C(O)c1c(NCc2cc(Cl)cc(Cl)c2)ccc2nccn12 ; O=C(O)c1c(NCc2cccc(OC(F)F)c2)ccc2nccn12]

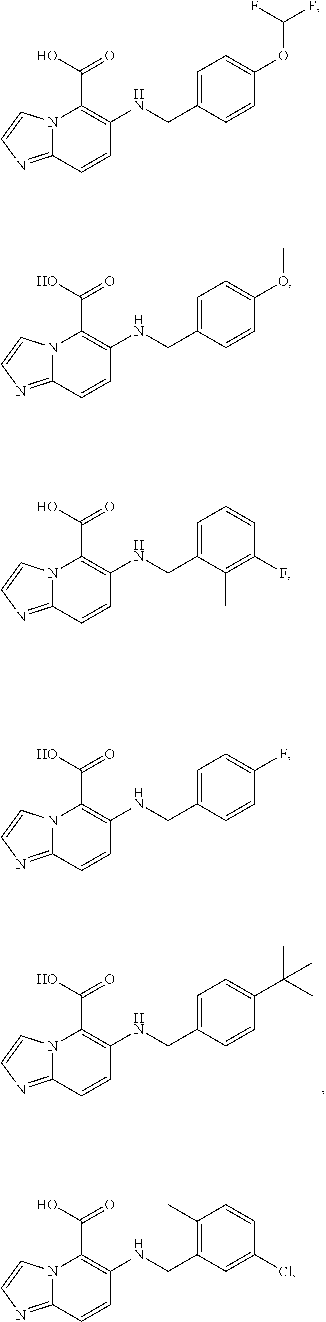 [SMILES: O=C(O)c1c(NCc2ccc(OC(F)F)cc2)ccc2nccn12 ; COc1ccc(CNc2ccc3nccn3c2C(=O)O)cc1 ; Cc1c(F)cccc1CNc1ccc2nccn2c1C(=O)O ; O=C(O)c1c(NCc2ccc(F)cc2)ccc2nccn12 ; CC(C)(C)c1ccc(CNc2ccc3nccn3c2C(=O)O)cc1 ; Cc1ccc(Cl)cc1CNc1ccc2nccn2c1C(=O)O]

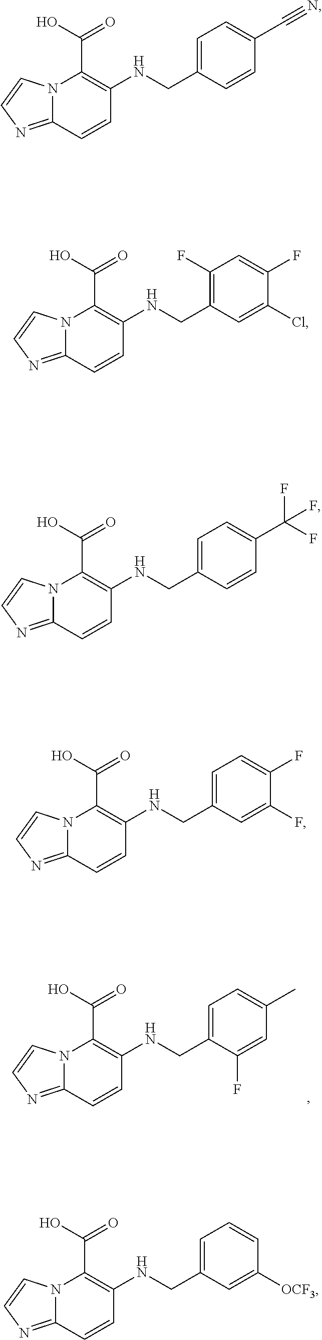 [SMILES: N#Cc1ccc(CNc2ccc3nccn3c2C(=O)O)cc1 ; O=C(O)c1c(NCc2cc(Cl)c(F)cc2F)ccc2nccn12 ; O=C(O)c1c(NCc2ccc(C(F)(F)F)cc2)ccc2nccn12 ; O=C(O)c1c(NCc2ccc(F)c(F)c2)ccc2nccn12 ; Cc1ccc(CNc2ccc3nccn3c2C(=O)O)c(F)c1 ; O=C(O)c1c(NCc2cccc(OC(F)(F)F)c2)ccc2nccn12]

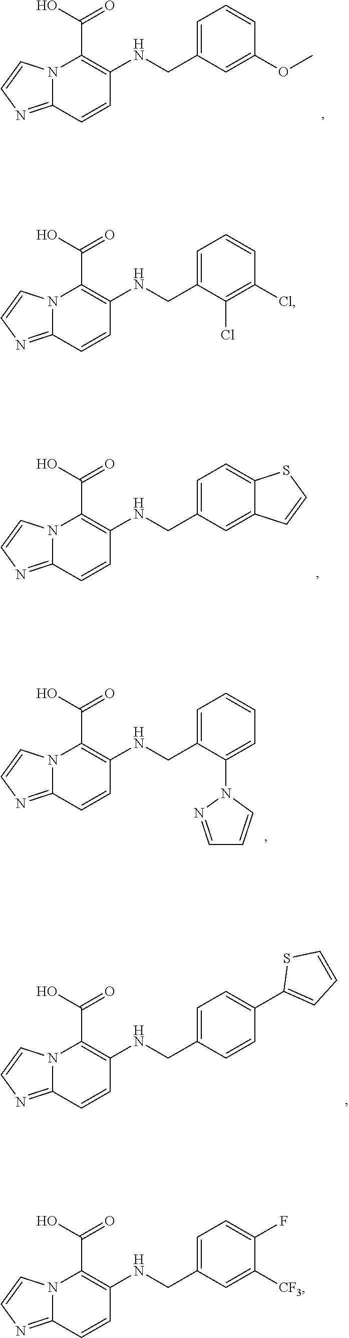 [SMILES: COc1cccc(CNc2ccc3nccn3c2C(=O)O)c1 ; O=C(O)c1c(NCc2cccc(Cl)c2Cl)ccc2nccn12 ; O=C(O)c1c(NCc2ccc3sccc3c2)ccc2nccn12 ; O=C(O)c1c(NCc2ccccc2-n2cccn2)ccc2nccn12 ; O=C(O)c1c(NCc2ccc(-c3cccs3)cc2)ccc2nccn12 ; O=C(O)c1c(NCc2ccc(F)c(C(F)(F)F)c2)ccc2nccn12]

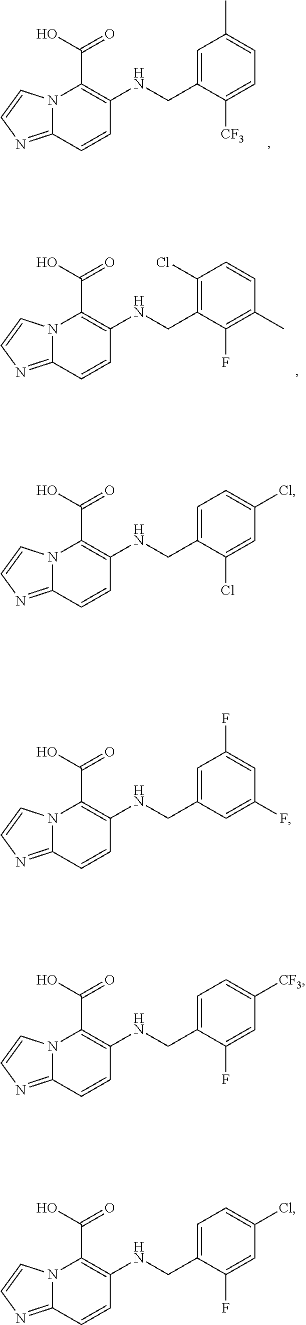 [SMILES: Cc1ccc(C(F)(F)F)c(CNc2ccc3nccn3c2C(=O)O)c1 ; Cc1ccc(Cl)c(CNc2ccc3nccn3c2C(=O)O)c1F ; O=C(O)c1c(NCc2ccc(Cl)cc2Cl)ccc2nccn12 ; O=C(O)c1c(NCc2cc(F)cc(F)c2)ccc2nccn12 ; O=C(O)c1c(NCc2ccc(C(F)(F)F)cc2F)ccc2nccn12 ; O=C(O)c1c(NCc2ccc(Cl)cc2F)ccc2nccn12]

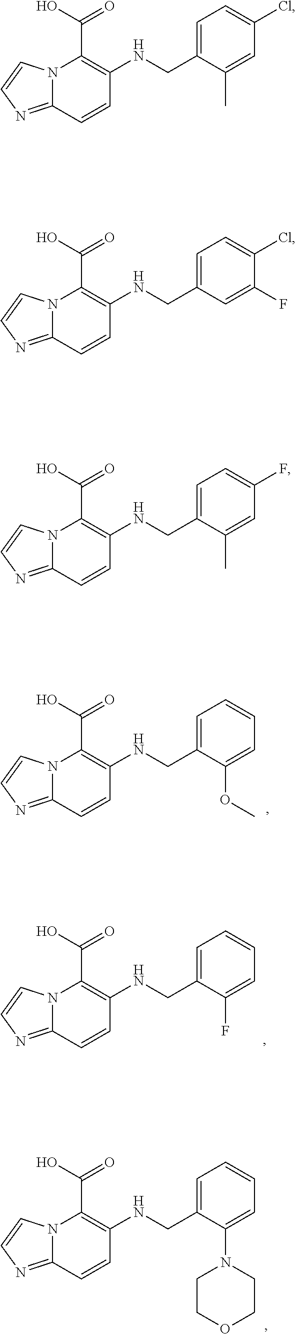 [SMILES: Cc1cc(Cl)ccc1CNc1ccc2nccn2c1C(=O)O ; O=C(O)c1c(NCc2ccc(Cl)c(F)c2)ccc2nccn12 ; Cc1cc(F)ccc1CNc1ccc2nccn2c1C(=O)O ; COc1ccccc1CNc1ccc2nccn2c1C(=O)O ; O=C(O)c1c(NCc2ccccc2F)ccc2nccn12 ; O=C(O)c1c(NCc2ccccc2N2CCOCC2)ccc2nccn12]

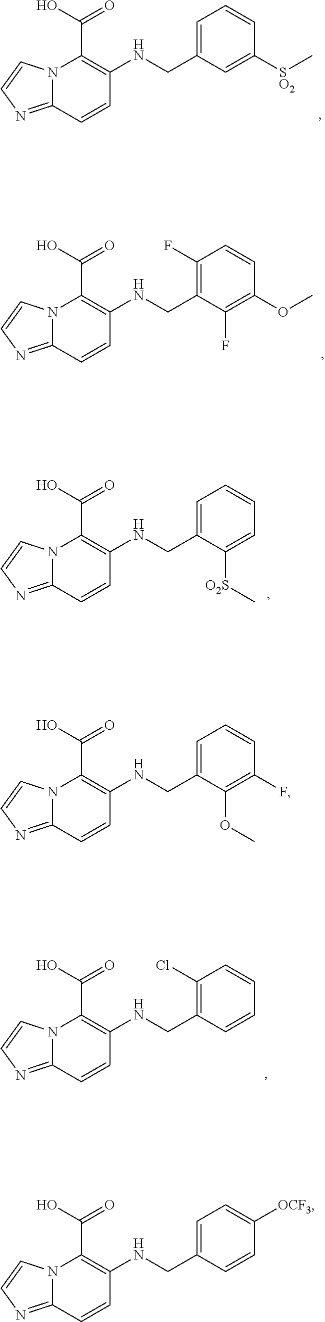 [SMILES: CS(=O)(=O)c1cccc(CNc2ccc3nccn3c2C(=O)O)c1 ; COc1ccc(F)c(CNc2ccc3nccn3c2C(=O)O)c1F ; CS(=O)(=O)c1ccccc1CNc1ccc2nccn2c1C(=O)O ; COc1c(F)cccc1CNc1ccc2nccn2c1C(=O)O ; O=C(O)c1c(NCc2ccccc2Cl)ccc2nccn12 ; O=C(O)c1c(NCc2ccc(OC(F)(F)F)cc2)ccc2nccn12]

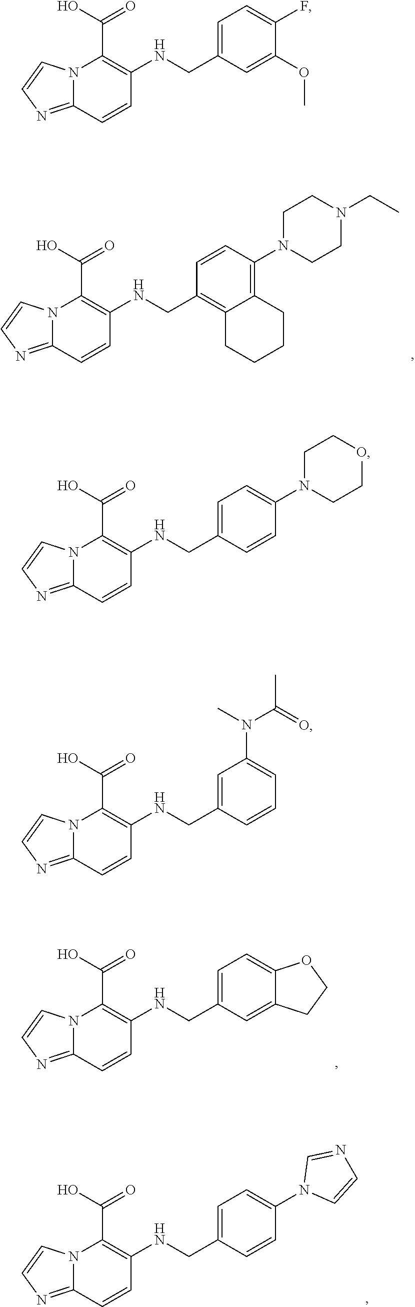 [SMILES: COc1cc(CNc2ccc3nccn3c2C(=O)O)ccc1F ; CCN1CCN(c2ccc(CNc3ccc4nccn4c3C(=O)O)c3c2CCCC3)CC1 ; O=C(O)c1c(NCc2ccc(N3CCOCC3)cc2)ccc2nccn12 ; CC(=O)N(C)c1cccc(CNc2ccc3nccn3c2C(=O)O)c1 ; O=C(O)c1c(NCc2ccc3c(c2)CCO3)ccc2nccn12 ; O=C(O)c1c(NCc2ccc(-n3ccnc3)cc2)ccc2nccn12]

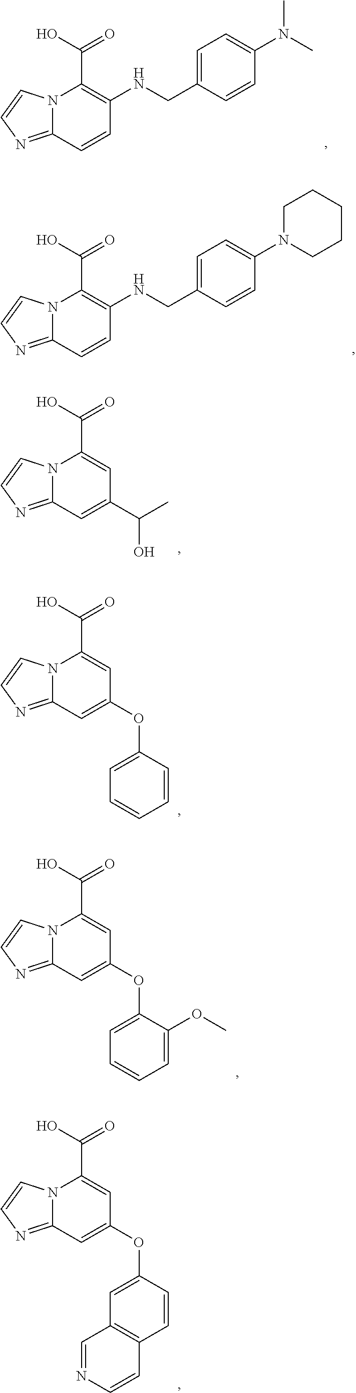 [SMILES: CN(C)c1ccc(CNc2ccc3nccn3c2C(=O)O)cc1 ; O=C(O)c1c(NCc2ccc(N3CCCCC3)cc2)ccc2nccn12 ; CC(O)c1cc(C(=O)O)n2ccnc2c1 ; O=C(O)c1cc(Oc2ccccc2)cc2nccn12 ; COc1ccccc1Oc1cc(C(=O)O)n2ccnc2c1 ; O=C(O)c1cc(Oc2ccc3ccncc3c2)cc2nccn12]

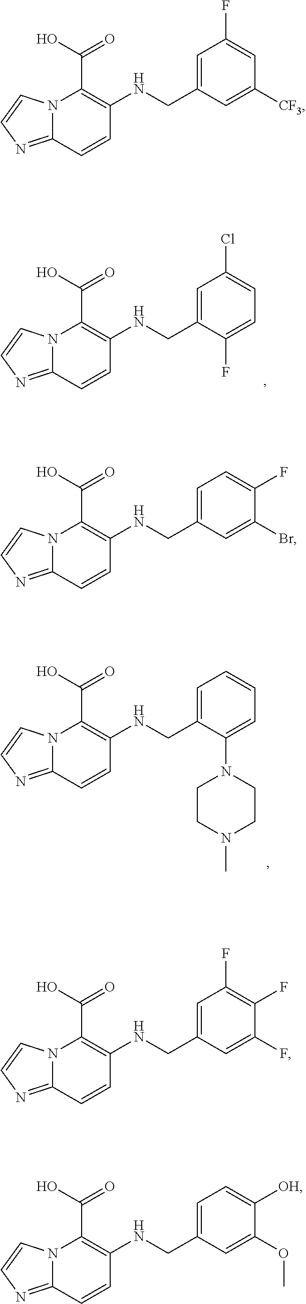 [SMILES: O=C(O)c1c(NCc2cc(F)cc(C(F)(F)F)c2)ccc2nccn12 ; O=C(O)c1c(NCc2cc(Cl)ccc2F)ccc2nccn12 ; O=C(O)c1c(NCc2ccc(F)c(Br)c2)ccc2nccn12 ; CN1CCN(c2ccccc2CNc2ccc3nccn3c2C(=O)O)CC1 ; O=C(O)c1c(NCc2cc(F)c(F)c(F)c2)ccc2nccn12 ; COc1cc(CNc2ccc3nccn3c2C(=O)O)ccc1O]

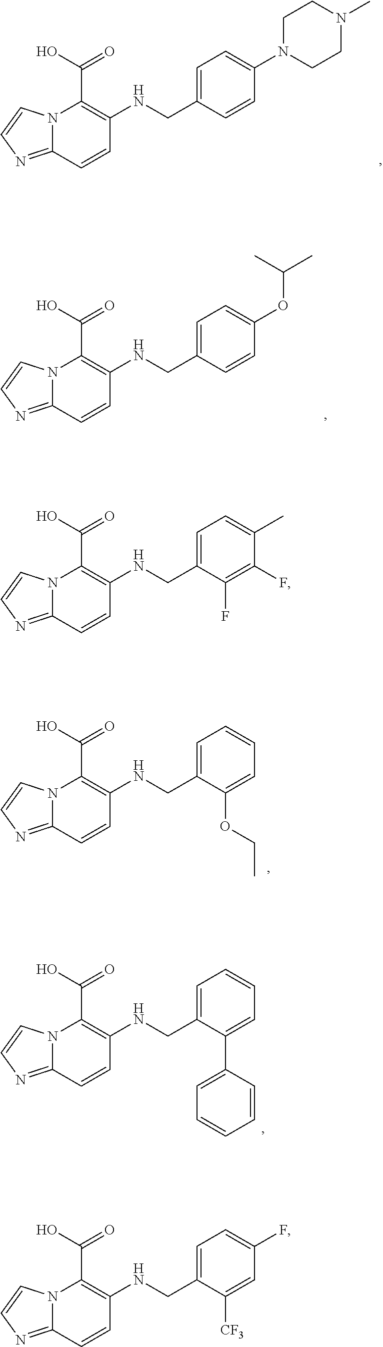 [SMILES: CN1CCN(c2ccc(CNc3ccc4nccn4c3C(=O)O)cc2)CC1 ; CC(C)Oc1ccc(CNc2ccc3nccn3c2C(=O)O)cc1 ; Cc1ccc(CNc2ccc3nccn3c2C(=O)O)c(F)c1F ; CCOc1ccccc1CNc1ccc2nccn2c1C(=O)O ; O=C(O)c1c(NCc2ccccc2-c2ccccc2)ccc2nccn12 ; O=C(O)c1c(NCc2ccc(F)cc2C(F)(F)F)ccc2nccn12]

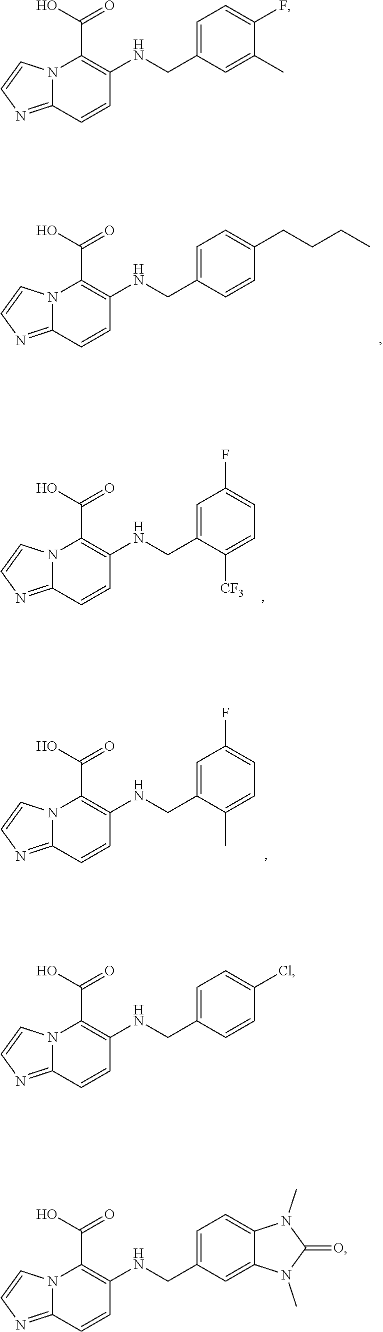 [SMILES: Cc1cc(CNc2ccc3nccn3c2C(=O)O)ccc1F ; CCCCc1ccc(CNc2ccc3nccn3c2C(=O)O)cc1 ; O=C(O)c1c(NCc2cc(F)ccc2C(F)(F)F)ccc2nccn12 ; Cc1ccc(F)cc1CNc1ccc2nccn2c1C(=O)O ; O=C(O)c1c(NCc2ccc(Cl)cc2)ccc2nccn12 ; Cn1c(=O)n(C)c2cc(CNc3ccc4nccn4c3C(=O)O)ccc21]

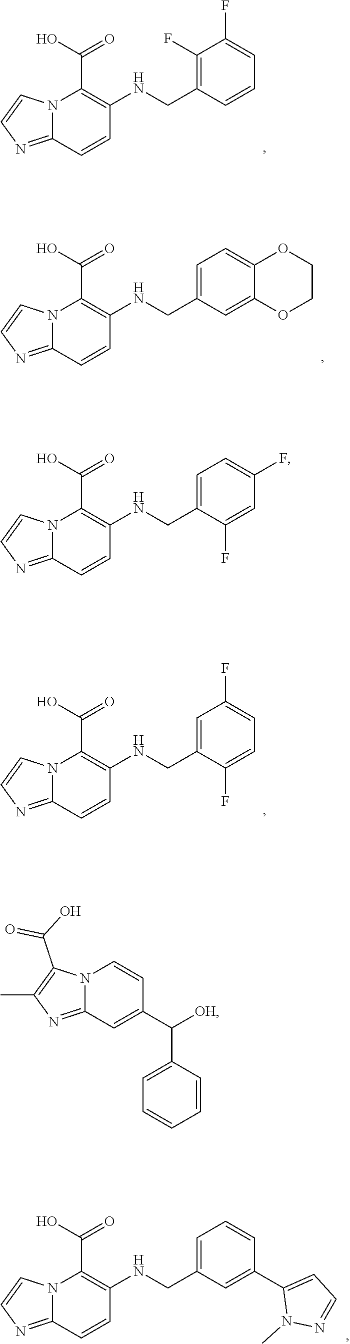 [SMILES: O=C(O)c1c(NCc2cccc(F)c2F)ccc2nccn12 ; O=C(O)c1c(NCc2ccc3c(c2)OCCO3)ccc2nccn12 ; O=C(O)c1c(NCc2ccc(F)cc2F)ccc2nccn12 ; O=C(O)c1c(NCc2cc(F)ccc2F)ccc2nccn12 ; Cc1nc2cc(C(O)c3ccccc3)ccn2c1C(=O)O ; Cn1nccc1-c1cccc(CNc2ccc3nccn3c2C(=O)O)c1]

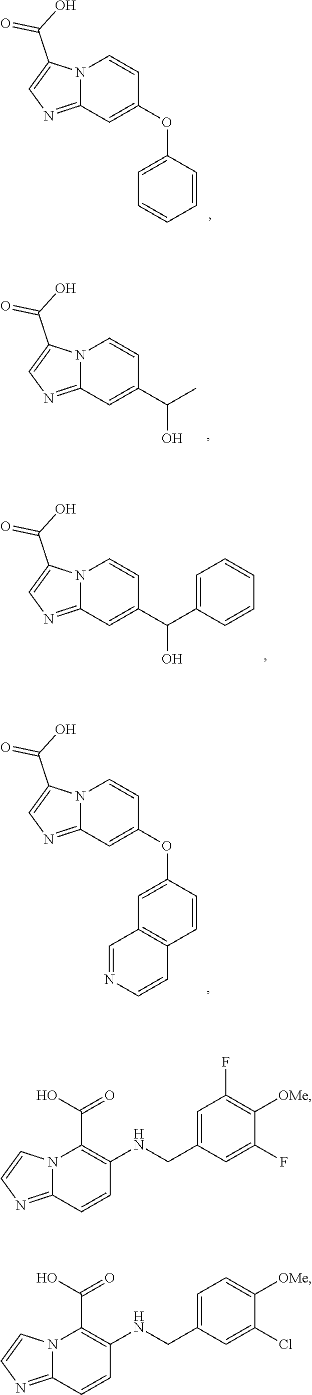 [SMILES: O=C(O)c1cnc2cc(Oc3ccccc3)ccn12 ; CC(O)c1ccn2c(C(=O)O)cnc2c1 ; O=C(O)c1cnc2cc(C(O)c3ccccc3)ccn12 ; O=C(O)c1cnc2cc(Oc3ccc4ccncc4c3)ccn12 ; COc1c(F)cc(CNc2ccc3nccn3c2C(=O)O)cc1F ; COc1ccc(CNc2ccc3nccn3c2C(=O)O)cc1Cl]

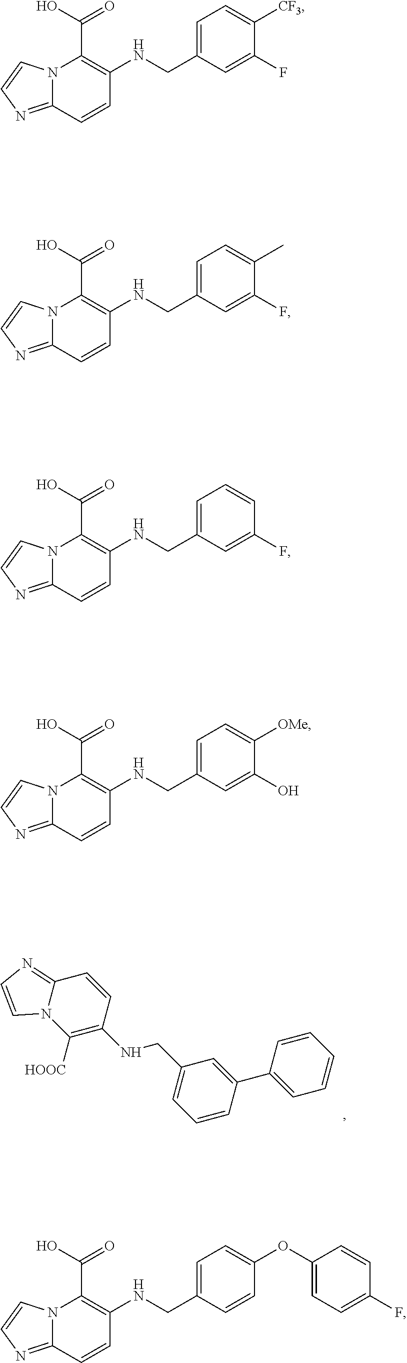 [SMILES: O=C(O)c1c(NCc2ccc(C(F)(F)F)c(F)c2)ccc2nccn12 ; Cc1ccc(CNc2ccc3nccn3c2C(=O)O)cc1F ; O=C(O)c1c(NCc2cccc(F)c2)ccc2nccn12 ; COc1ccc(CNc2ccc3nccn3c2C(=O)O)cc1O ; O=C(O)c1c(NCc2cccc(-c3ccccc3)c2)ccc2nccn12 ; O=C(O)c1c(NCc2ccc(Oc3ccc(F)cc3)cc2)ccc2nccn12]

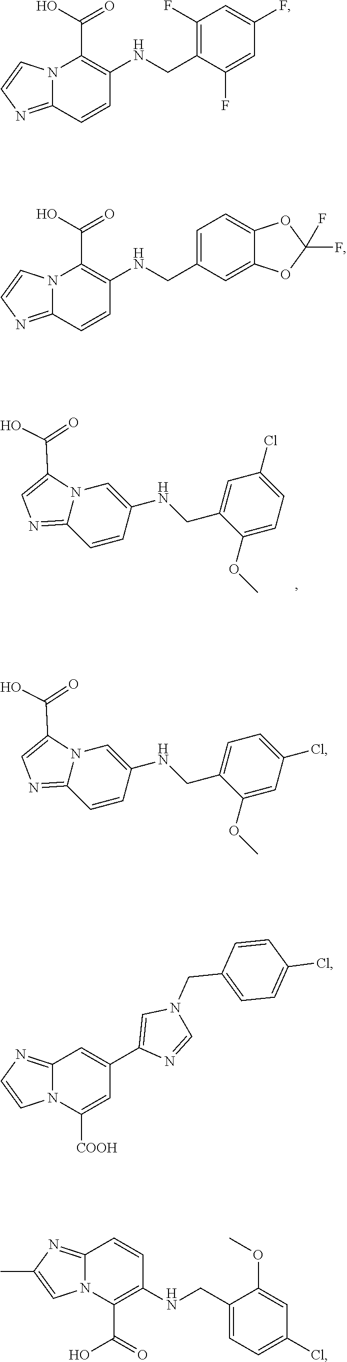 [SMILES: O=C(O)c1c(NCc2c(F)cc(F)cc2F)ccc2nccn12 ; O=C(O)c1c(NCc2ccc3c(c2)OC(F)(F)O3)ccc2nccn12 ; COc1ccc(Cl)cc1CNc1ccc2ncc(C(=O)O)n2c1 ; COc1cc(Cl)ccc1CNc1ccc2ncc(C(=O)O)n2c1 ; O=C(O)c1cc(-c2cn(Cc3ccc(Cl)cc3)cn2)cc2nccn12 ; COc1cc(Cl)ccc1CNc1ccc2nc(C)cn2c1C(=O)O]

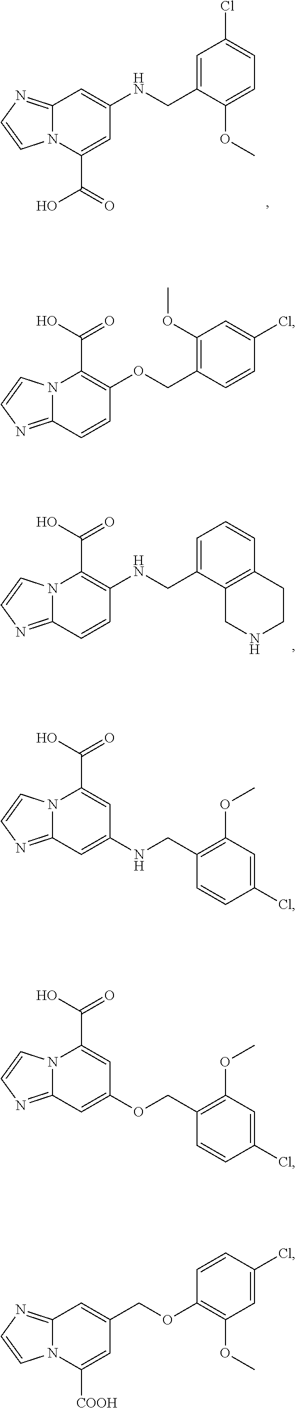 [SMILES: COc1ccc(Cl)cc1CNc1cc(C(=O)O)n2ccnc2c1 ; COc1cc(Cl)ccc1COc1ccc2nccn2c1C(=O)O ; O=C(O)c1c(NCc2cccc3c2CNCC3)ccc2nccn12 ; COc1cc(Cl)ccc1CNc1cc(C(=O)O)n2ccnc2c1 ; COc1cc(Cl)ccc1COc1cc(C(=O)O)n2ccnc2c1 ; COc1cc(Cl)ccc1OCc1cc(C(=O)O)n2ccnc2c1]

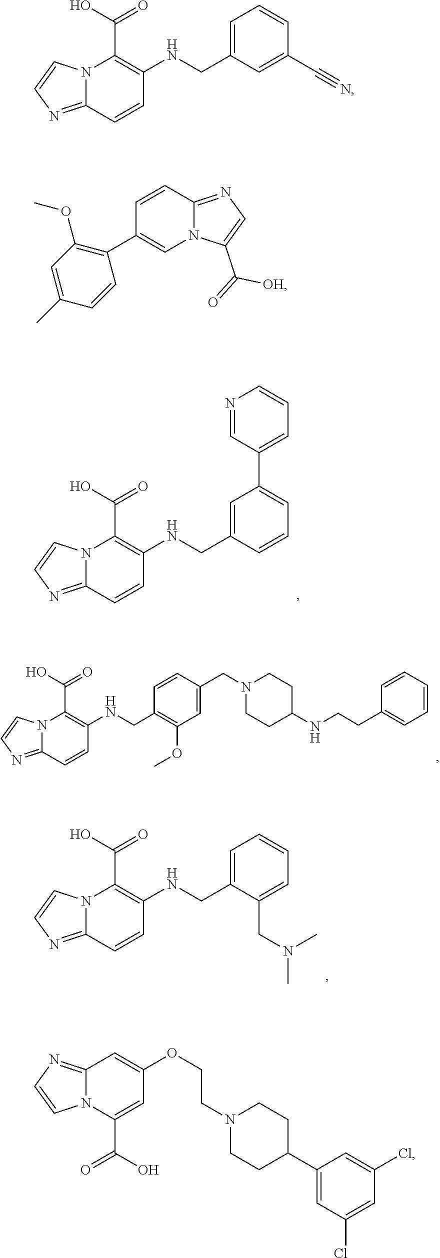 [SMILES: N#Cc1cccc(CNc2ccc3nccn3c2C(=O)O)c1 ; COc1cc(C)ccc1-c1ccc2ncc(C(=O)O)n2c1 ; O=C(O)c1c(NCc2cccc(-c3cccnc3)c2)ccc2nccn12 ; COc1cc(CN2CCC(NCCc3ccccc3)CC2)ccc1CNc1ccc2nccn2c1C(=O)O ; CN(C)Cc1ccccc1CNc1ccc2nccn2c1C(=O)O ; O=C(O)c1cc(OCCN2CCC(c3cc(Cl)cc(Cl)c3)CC2)cc2nccn12]

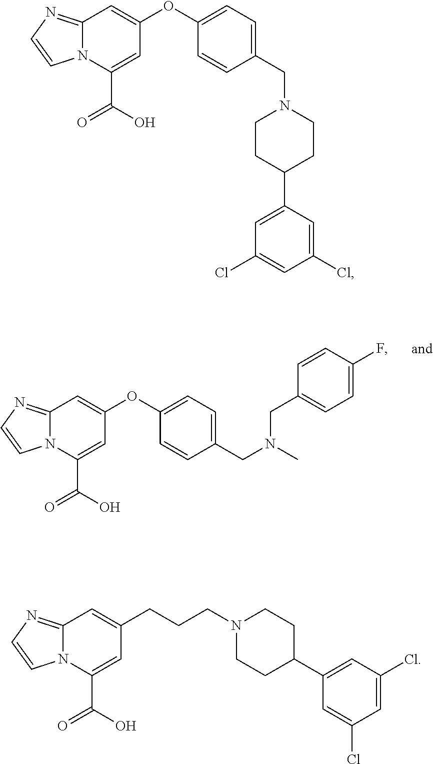 [SMILES: O=C(O)c1cc(Oc2ccc(CN3CCC(c4cc(Cl)cc(Cl)c4)CC3)cc2)cc2nccn12 ; CN(Cc1ccc(F)cc1)Cc1ccc(Oc2cc(C(=O)O)n3ccnc3c2)cc1 ; O=C(O)c1cc(CCCN2CCC(c3cc(Cl)cc(Cl)c3)CC2)cc2nccn12]

18. A pharmaceutical composition comprising a therapeutically effective amount of a compound of formula (I) of claim 1, and a pharmaceutically acceptable excipient.

19-27. (canceled)
